(19) United States
(12) Patent Application Publication
(10) Pub. No.: US 2007/0060589 A1
(43) Pub. Date: Mar. 15, 2007

(54) Inhibitors of protein arginine methyl transferases

(76) Inventors: Ashok Vinayak Purandare, Pennington, NJ (US); Zhong Chen, Princeton, NJ (US)

Correspondence Address: LOUIS J. WILLE;BRISTOL-MYERS SQUIBB COMPANY, PATENT DEPARTMENT, P O BOX 4000, PRINCETON, NJ, 08543-4000, US

(21) Appl. No.: 11/312,812

(22) Filed: Dec. 20, 2005

Related U.S. Application Data
(60) Provisional application No. 60/637,893, filed on Dec. 21, 2004.

(51) Int. Cl. A61K 31/506; A61K 31/501; A61K 31/497; A61K 31/4709; A61K 31/4545; A61K 31/454; C07D 417/02; C07D 413/02; C07D 403/02

(52) U.S. Cl. 514/252.05; 514/310; 514/326; 514/255.05; 514/256; 514/318; 544/238; 544/333; 544/405; 546/146; 546/194; 546/209

(57) ABSTRACT

A compound of formula I, or a stereoisomer, a tautomer, a pharmaceutically acceptable salt or solvate thereof, 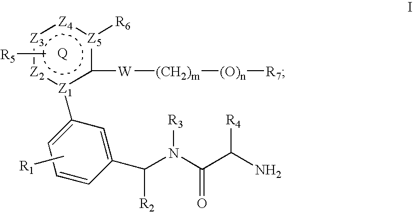 methods of using such compounds in the treatment of hyperproliferative, inflammatory, infectious, and immunoregulatory disorders and diseases; and to pharmaceutical compositions containing such compounds.

### CROSS-REFERENCE TO RELATED APPLICATIONS

The present application claims the priority benefit of U.S. Provisional Application No. 60/637,893, filed Dec. 21, 2004 which is expressly incorporated fully herein by reference

### FIELD OF THE INVENTION

The present invention relates to novel compounds which are inhibitors of Protein ARginine Methyl Transferases (PRMTs), to methods of using such compounds for inhibiting protein methyl transferases in the treatment of hyperproliferative, inflammatory, infectious, and immunoregulatory disorders and diseases, and to pharmaceutical compositions containing such compounds.

The invention also encompasses pharmaceutical compositions containing these compounds. The compounds and pharmaceutical compositions of the invention are particularly well suited as inhibitors of protein methyl transferases and, consequently, can be advantageously used as therapeutic agents for the treatment of, including cancer, asthma, COPD, and allergic diseases; rheumatoid arthritis, atherosclerosis, and psoriasis; solid organ transplant rejection, osteoarthritis, and inflammatory bowel syndrome. This invention also relates to methods of using the compounds of this invention alone or in combination with other pharmaceutically active agents.

### BACKGROUND OF THE INVENTION

PRMTs are enzymes that catalyze the transfer of methyl groups from S-Adenosyl-L-Methionine (SAM) to specific arginine residues of proteins. Arginine methylation of proteins has been implicated to play roles in pre-mRNA splicing, nucleo-cytoplasmic RNA transport, signal transduction and transcriptional activation. To date seven family members have been identified (PRMTs 1-7) in mammalian cells and they each appear to have distinct substrate preferences. PRMT1 has been shown to methylate Histone H4 and this results in activation of transcription. Coactivator Associated Arginine Methyltransferase-I (CARM-1) (also called PRMT-4) has been shown to methylate Histone H3 both in vitro and in vivo and it is speculated that this modification positively affects chromatin remodeling and thus transcriptional activation. CARM-1 has also been shown to play a co-activator role in gene transcription mediated by the nuclear hormone receptor (NHR) family of transcription factors. In this context, CARM-1 displays an absolute requirement for the presence a member of the NHR co-activator family of proteins (SRC-1, GRIP-1 or AIB1) in order to enhance transcriptional activation by the androgen receptor (AR) or estrogen receptor (ER). It is also able to co-operate with p300/CBP and PRMT-1 in NHR dependent transcription. In the resulting transcriptionally active complex, CARM-1 can methylate both p300/CBP and the NHR co-activator(s). The fact that mutations of critical residues in the catalytic domain compromise the co-activator function of CARM-1 suggests that the integrity of its methyl transferase domain is not dispensable for its co-activator function. Additionally, CARM-1 has been implicated to play a key role in mediating signal transduction pathways among CREB Binding Protein (CBP), p53 and the muscle enhancer factor-1 (MEF-1).

In addition to its role as a coactivator in NHR mediated signaling, CARM-1 has also been implicated as the PRMT responsible for methylation of PABP (Poly-A-binding protein) and HuR (a member of the Hu family of protein). PABP and HuR have been shown to bind to messenger RNAs (e.g. fos, Cox-2, β-catenin) containing the AUUUUA sequence in the 3′ untranslated region thereby stabilizing the message and leading to increased translation in the cytoplasm.

Accordingly, CARM-1 may serve as an oncogene because its ability to regulate transcription, modulate chromatin organization and increase the half-life of specific mRNAs. Upon comparison of a number of matched tumor and normal tissues (lung, colon and breast), we found that CARM-1 was over-expressed in the tumor specimens compared to normal tissue (WO03102143). Moreover, Hong et al (Cancer, 101 (1), 83-89 (2004)) reports that CARM-1 levels are significantly higher in PIN (Prostatic intraepithelial neoplasia) and prostatic adenocarcinoma specimens from patients compared to benign prostate tissue specimens. See “Aberrant expression of CARM-1, a transcriptional coactivator of Androgen receptor in the development of prostate carcinoma and androgen independent status. In twelve patients with androgen independent prostatic adenocarcinoma, the expression of CARM-1 was significantly increased when compared to patients without previous hormonal treatment.

Furthermore, HuR, one of the substrates of CARM-1, has also been implicated in cancer (Li et al, J Biol Chem, 277, 44623 (2002), Erkinheimo et al, Cancer Res, 63, 7591 (2003)) HuR is a nuclear protein but is predominantly cytoplasmic in tumor cells. Increased cytoplasmic presence predicts a poor prognosis. Thus, methylation by CARM-1 may be responsible for the cytoplasmic presence of HuR which resulted in the increased stabilization of mRNAs of genes implicated in cancer (e.g. fos, Cox-2, β-catenin) (Denkert et al, Cancer Res, 64, 189 (2004). Accordingly, it is an attractive therapeutic option for cancer patients to inhibit the enzymatic function of CARM-1 by using a small organic molecule.

### SUMMARY OF THE INVENTION

The present invention provides a compound of the following formula I, or a stereoisomer, a tautomer, a pharmaceutically acceptable salt or solvate thereof, which compounds are especially useful as inhibitors of PRMTs and/or CARM-1;

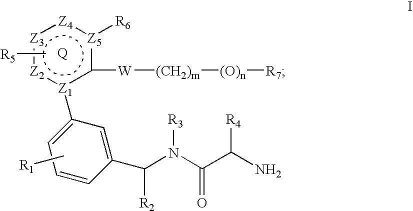

wherein:
Ring Q is phenyl; 5-membered heteroaryl, in which Z₄ is a bond, Z₁, Z₂, Z₃ and Z₅ are each independently C, N, O, or S, and at least one of Z₁, Z₂, Z₃ and Z₅ is a heteroatom selected from N, O and S; or 6-membered heteroaryl, in which Z₁, Z₂, Z₃, Z₄ and Z₅ are each independently C, or N, and at least one of Z₁, Z₂, Z₃, Z₄ and Z₅ is N;
W is —C(═O)NR₈—, —NR₉C(═O)—, —NR₉C(═O)NR₈—, 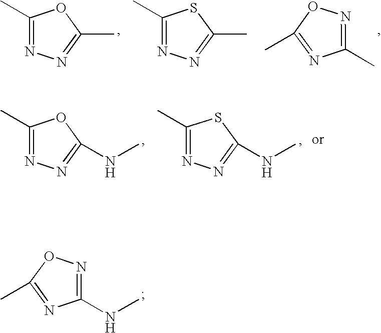
alternatively, W—(CH₂)—(O)n-R₇ is 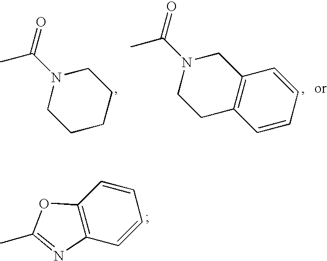
R₁ is H, halogen, CN, alkyl or substituted alkyl, O—C₁-C₄ alkyl, S—C₁-C₄ alkyl, or SO₂—C₁-C₄ alkyl;
R₂ is H, or C₁-C₄ alkyl;
R₃ is H, Me or Et, or optionally R₃ together with R₄ may form a 5- or 6-membered heterocycle
R₄ is H, Me, Et, iso-propyl, CH₂Ph, OH, or OPh, or optionally R₄ together with R₃ may form a 5- or 6-membered heterocycle;
R₅ is nil, H, Me, Et, propyl, iso-propyl, OMe, OEt, SMe, SO₂Me, CF₃, or OCF₃;
R₆ is nil, H, Me, or Et, or optionally R₆ together with R₈ may form a 5- or 6-membered heterocycle;
R₇ is cycloalkyl or substituted cycloalkyl, heterocycle or substituted heterocycle, or aryl or substituted aryl;
R₈ is H, or Me, or optionally R₈ together with R₆ may form a 5- or 6-membered heterocycle;
   or alternatively R₈ together with R₇ may form a 5- or 6-membered heterocycle or substituted heterocycle;
R₉ is H, or Me;
m is 0, 1, 2 or 3;
n is 0 or 1; and
p is 1, 2 or 3.

The present invention further provides a compound of the following formula I, or a stereoisomer, a tautomer, a pharmaceutically acceptable salt or solvate thereof, which compounds are especially useful as inhibitors of PRMTs and/or CARM-1.

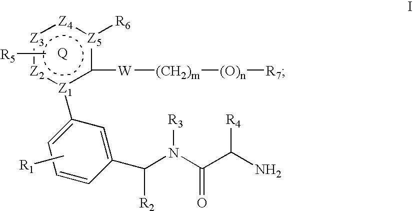

As used in formula I, and throughout the specification, the symbols have the following meanings unless otherwise indicated, and are, for each occurrence, independently selected:
Ring Q is phenyl; 5-membered heteroaryl, in which Z₄ is a bond, Z₁, Z₂, Z₃ and Z₅ are each independently C, N, O, or S, and at least one of Z₁, Z₂, Z₃ and Z₅ is a heteroatom selected from N, O and S; or 6-membered heteroaryl, in which Z₁, Z₂, Z₃, Z₄ and Z₅ are each independently C, or N, and at least one of Z₁, Z₂, Z₃, Z₄ and Z₅ is N;
W is —C(═O)NR₈—, —NR₉C(═O)—, 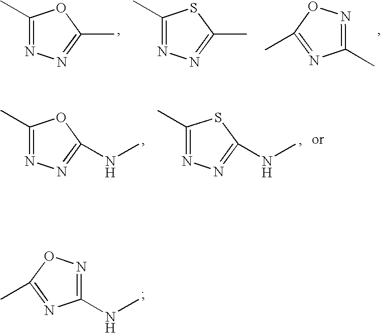
R₁ is H, halogen, CN, alkyl or substituted alkyl, O—C₁-C₄ alkyl, S—C₁-C₄ alkyl, or SO₂—C₁-C₄ alkyl;
R₂ is H or C₁-C₄ alkyl;
R₃ is H, Me or Et, or optionally R₃ together with R₄ may form a 5- or 6-membered heterocycle
R₄ is H, Me, Et, iso-propyl, CH₂Ph, OH, or OPh, or optionally R₄ together with R₃ may form a 5- or 6-membered heterocycle;
R₅ is nil, H, Me, Et, propyl, iso-propyl, OMe, OEt, SMe, SO₂Me, CF₃, or OCF₃;
R₆ is nil, H, Me, or Et, or optionally R₆ together with R₈ may form a 5- or 6-membered heterocycle;
R₇ is cycloalkyl or substituted cycloalkyl, heterocycle or substituted heterocycle, or aryl or substituted aryl;
R₈ is H, or Me, or optionally R₈ together with R₆ may form a 5- or 6-membered heterocycle;
   or alternatively R₈ together with R₇ may form a 5- or 6-membered heterocycle or substituted heterocycle;
R₉ is H, or Me;
m is 0, 1, 2 or 3;
n is 0 or 1; and
p is 1, 2 or 3.

The present invention also relates to methods of using compounds of formula I in the treatment of hyperproliferative, inflammatory, infectious, and immunoregulatory disorders and diseases, and to pharmaceutical compositions containing such compounds.

The present invention provides pharmaceutical compositions comprising a pharmaceutically acceptable carrier and a therapeutically effective amount of at least one of the compounds of the present invention or a pharmaceutically acceptable salt or prodrug form thereof.

The present invention provides pharmaceutical compositions comprising a pharmaceutically acceptable carrier and an amount of a compound of formula (I) to provide a therapeutically effective amount of at least one of the compounds of the present invention or a pharmaceutically acceptable salt or prodrug form thereof.

The present invention provides a method of treating cancers, wherein the cancer is selected from breast cancer, lung cancer, ovarian cancer, prostate cancer, leukemia, lymphoma, glioblastoma, brain cancer, melanoma, and colon cancer.

The present invention provides novel compounds for use in therapy.

The present invention provides the use of novel compounds for the manufacture of a medicament for the treatment of oncological or immunological diseases.

In another embodiment, the present invention is directed to compounds of formula I wherein ring Q is 5-membered heteroaryl, in which Z₄ is a bond, Z₁, Z₂, Z₃ and Z₅ are each independently C, N, O, or S, and at least one of Z₁, Z₂, Z₃ and Z₅ is a heteroatom selected from N, O and S; or 6-membered heteroaryl, in which Z₁, Z₂, Z₃, Z₄ and Z₅ are each independently C, or N, and at least one of Z₁, Z₂, Z₃, Z₄ and Z₅ is N.

In another embodiment, the present invention is directed to compounds of formula I wherein ring Q is 5-membered heteroaryl, in which Z₄ is a bond, Z₁, Z₂, Z₃ and Z₅ are each independently C, N, O, or S, and at least one of Z₁, Z₂, Z₃ and Z₅ is a heteroatom selected from N, O and S.

In another embodiment, the present invention is directed to compounds of formula I wherein Z₄ is a bond, Z₁ and Z₂, are each independently N; Z₃ and Z₅ are each independently C.

In another embodiment, the present invention is directed to compounds of formula I wherein R₇ is aryl or substituted aryl.

In another embodiment, the present invention is directed to compounds of formula I wherein R₇ is phenyl or substituted phenyl.

In another embodiment, the present invention is directed to compounds of formula I wherein W is C(═O)NR₈—.

In another embodiment, the present invention is directed to compounds of formula I wherein W is

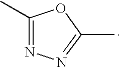

In another embodiment, the present invention is directed to compounds of formula I wherein W is

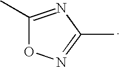

In another embodiment, the present invention is directed to compounds of formula I wherein R₂ is H, R₃ is H, R₄ is Me, Et, OH, or Ph.

In another embodiment, the present invention is directed to compounds of formula I wherein R₄ is Me.

In another embodiment, the present invention is directed to compounds of formula I wherein R₄ is (S—) Me.

In another embodiment, the present invention is directed to compounds of formula I wherein W is —C(═O)NR₈—.

In another embodiment, the present invention is directed to compounds of formula I wherein Q is

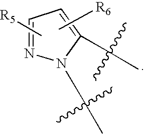

In another embodiment, the present invention is directed to compounds of formula I wherein Q is

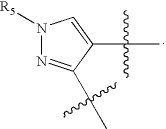

In another embodiment, the present invention is directed to compounds of formula I wherein Q is

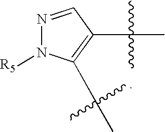

In another embodiment, the present invention is directed to compounds of formula I wherein Q is

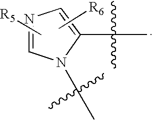

In another embodiment, the present invention is directed to compounds of formula I wherein Q is

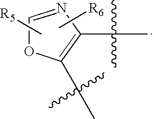

In another embodiment, the present invention is directed to compounds of formula I wherein Q is

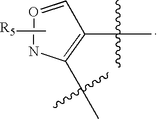

In another embodiment, the present invention is directed to compounds of formula I wherein Q is

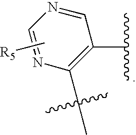

The invention may be embodied in other specific forms without departing from the spirit or essential attributes thereof. This invention also encompasses all combinations of preferred aspects of the invention noted herein. It is understood that any and all embodiments of the present invention may be taken in conjunction with any other embodiment to describe additional even more preferred embodiments of the present invention. Furthermore, any elements of an embodiment are meant to be combined with any and all other elements from any of the embodiments to describe additional embodiments.

### FURTHER DESCRIPTION OF THE INVENTION

### Definitions

The following are definitions of terms used in the present specification. The initial definition provided for a group or term herein applies to that group or term throughout the present specification individually or as part of another group, unless otherwise indicated.

The terms “alkyl” and “alk” refers to a straight or branched chain alkane (hydrocarbon) radical containing from 1 to 12 carbon atoms, preferably 1 to 6 carbon atoms. Exemplary “alkyl” groups include methyl, ethyl, propyl, isopropyl, n-butyl, t-butyl, isobutyl pentyl, hexyl, isohexyl, heptyl, 4,4-dimethylpentyl, octyl, 2,2,4-trimethylpentyl, nonyl, decyl, undecyl, dodecyl, and the like. The term “C₁-C₄ alkyl” refers to a straight or branched chain alkane (hydrocarbon) radical containing from 1 to 4 carbon atoms, such as methyl, ethyl, propyl, isopropyl, n-butyl, t-butyl, and isobutyl. “Haloalkyl” refers to an alkyl group substituted with one or more halogen. “Substituted alkyl” refers to an alkyl group substituted with one or more substituents, preferably 1 to 4 substituents, at any available point of attachment. Exemplary substituents include but are not limited to one or more of the following groups: hydrogen, halogen (e.g., a single halogen substituent or multiple halo substitutents forming, in the latter case, groups such as CF₃ or an alkyl group bearing Cl₃), cyano, nitro, cycloalkyl, alkenyl, cycloalkenyl, alkynyl, heterocycle, aryl, ORₐ, SRₐ, S(═O)Rₑ, S(═O)₂Rₑ, P(═O)₂Rₑ, S(═O)₂ORₑ, P(═O)₂ORₑ, NR_{b}R_{c}, NR_{b}S(═O)₂Rₑ, NR_{b}P(═O)₂Rₑ, S(═O)₂NR_{b}R_{c}, P(═O)₂NR_{b}R_{c}, C(═O)ORₑ, C(═O)Rₐ, C(═O)NR_{b}R_{c}, OC(═O)Rₐ, OC(═O)NR_{b}R_{c}, NR_{b}C(═O)ORₑ, NR_{d}C(═O)NR_{b}R_{c}, NR_{d}S(═O)₂NR_{b}R_{c}, NR_{d}P(═O)₂NR_{b}R_{c}, NR_{b}C(═O)Rₐ, or NR_{b}P(═O)₂Rₑ, wherein Rₐ is hydrogen, alkyl, cycloalkyl, alkenyl, cycloalkenyl, alkynyl, heterocycle, or aryl; R_{b}, R_{c} and R_{d} are independently hydrogen, alkyl, cycloalkyl, heterocycle, aryl, or said R_{b} and R_{c} together with the N to which they are bonded optionally form a heterocycle; and Rₑ is alkyl, cycloalkyl, alkenyl, cycloalkenyl, alkynyl, heterocycle, or aryl. In the aforementioned exemplary substitutents, groups such as alkyl, cycloalkyl, alkenyl, alkynyl, cycloalkenyl, heterocycle and aryl can themselves be optionally substituted.

The term “alkenyl” refers to a straight or branched chain hydrocarbon radical containing from 2 to 12 carbon atoms and at least one carbon-carbon double bond. Exemplary such groups include ethenyl or allyl. “Substituted alkenyl” refers to an alkenyl group substituted with one or more substituents, preferably 1 to 4 substituents, at any available point of attachment. Exemplary substituents include, but are not limited to, alkyl or substituted alkyl, as well as those groups recited above as exemplary alkyl substituents.

The term “alkynyl” refers to a straight or branched chain hydrocarbon radical containing from 2 to 12 carbon atoms and at least one carbon to carbon triple bond. Exemplary such groups include ethynyl. “Substituted alkynyl” refers to an alkynyl group substituted with one or more substituents, preferably 1 to 4 substituents, at any available point of attachment. Exemplary substituents include, but are not limited to, alkyl or substituted alkyl, as well as those groups recited above as exemplary alkyl substituents.

The term “cycloalkyl” refers to a fully saturated cyclic hydrocarbon group containing from 1 to 4 rings and 3 to 8 carbons per ring. Exemplary such groups include cyclopropyl, cyclobutyl, cyclopentyl, cyclohexyl, cycloheptyl, etc. “Substituted cycloalkyl” refers to a cycloalkyl group substituted with one or more substituents, preferably 1 to 4 substituents, at any available point of attachment. Exemplary substituents include, but are not limited to, nitro, cyano, alkyl or substituted alkyl, as well as those groups recited above as exemplary alkyl substituents. Exemplary substituents also include spiro-attached or fused cyclic substituents, especially spiro-attached cycloalkyl, spiro-attached cycloalkenyl, spiro-attached heterocycle (excluding heteroaryl), fused cycloalkyl, fused cycloalkenyl, fused heterocycle, or fused aryl, where the aforementioned cycloalkyl, cycloalkenyl, heterocycle and aryl substitutents can themselves be optionally substituted.

The term “cycloalkenyl” refers to a partially unsaturated cyclic hydrocarbon group containing 1 to 4 rings and 3 to 8 carbons per ring. Exemplary such groups include cyclobutenyl, cyclopentenyl, cyclohexenyl, etc. “Substituted cycloalkenyl” refers to a cycloalkenyl group substituted with one more substituents, preferably 1 to 4 substituents, at any available point of attachment. Exemplary substituents include but are not limited to nitro, cyano, alkyl or substituted alkyl, as well as those groups recited above as exemplary alkyl substituents. Exemplary substituents also include spiro-attached or fused cyclic substituents, especially spiro-attached cycloalkyl, spiro-attached cycloalkenyl, spiro-attached heterocycle (excluding heteroaryl), fused cycloalkyl, fused cycloalkenyl, fused heterocycle, or fused aryl, where the aforementioned cycloalkyl, cycloalkenyl, heterocycle and aryl substituents can themselves be optionally substituted.

The term “aryl” refers to cyclic, aromatic hydrocarbon groups that have 1 to 5 aromatic rings, especially monocyclic or bicyclic groups such as phenyl, biphenyl or naphthyl. Where containing two or more aromatic rings (bicyclic, etc.), the aromatic rings of the aryl group may be joined at a single point (e.g., biphenyl), or fused (e.g., naphthyl, phenanthrenyl and the like). “Substituted aryl” refers to an aryl group substituted by one or more substituents, preferably 1 to 3 substituents, at any point of attachment. Exemplary substituents include, but are not limited to, nitro, cycloalkyl or substituted cycloalkyl, cycloalkenyl or substituted cycloalkenyl, cyano, alkyl or substituted alkyl, as well as those groups recited above as exemplary alkyl substituents. Exemplary substituents also include fused cyclic groups, especially fused cycloalkyl, fused cycloalkenyl, fused heterocycle, or fused aryl, where the aforementioned cycloalkyl, cycloalkenyl, heterocycle and aryl substituents can themselves be optionally substituted. Exemplary aryl and phenyl substituents include but are not limited to one or more of the following groups: hydrogen, alkyl, haloalkyl, halogen (e.g., a single halogen substituent or multiple halo substitutents forming, in the latter case, groups such as CF₃ or an alkyl group bearing Cl₃), cyano, nitro, cycloalkyl, alkenyl, cycloalkenyl, alkynyl, heterocycle, aryl, ORₐ, SRₐ, S(═O)Rₑ, S(═O)₂Rₑ, P(═O)₂Rₑ, S(═O)₂ORₑ, P(═O)₂ORₑ, NR_{b}R_{c}, NR_{b}S(═O)₂Rₑ, NR_{b}P(═O)₂Rₑ, S(═O)₂NR_{b}R_{c}, P(═O)₂NR_{b}R_{c}, C(═O)ORₑ, C(═O)Rₐ, C(═O)NR_{b}R_{c}, OC(═O)Rₐ, OC(═O)NR_{b}R_{c}, NR_{b}C(═O)ORₑ, NR_{d}C(═O)NR_{b}R_{c}, NR_{d}S(═O)₂NR_{b}R_{c}, NR_{d}P(═O)₂NR_{b}R_{c}, NR_{b}C(═O)Rₐ, or NR_{b}P(═O)₂Rₑ, wherein Rₐ is hydrogen, alkyl, cycloalkyl, alkenyl, cycloalkenyl, alkynyl, heterocycle, or aryl; R_{b}, R_{c} and R_{d} are independently hydrogen, alkyl, cycloalkyl, heterocycle, aryl, or said R_{b} and R_{c} together with the N to which they are bonded optionally form a heterocycle; and Rₑ is alkyl, cycloalkyl, alkenyl, cycloalkenyl, alkynyl, heterocycle, or aryl. In the aforementioned exemplary substitutents, groups such as alkyl, cycloalkyl, alkenyl, alkynyl, cycloalkenyl, heterocycle and aryl can themselves be optionally substituted.

The terms “heterocycle” and “heterocyclic” refer to fully saturated, or partially or fully unsaturated, including aromatic (i.e., “heteroaryl”) cyclic groups (for example, 4 to 7 membered monocyclic, 7 to 11 membered bicyclic, or 10 to 16 membered tricyclic ring systems) which have at least one heteroatom in at least one carbon atom-containing ring. Each ring of the heterocyclic group containing a heteroatom may have 1, 2, 3, or 4 heteroatoms selected from nitrogen atoms, oxygen atoms and/or sulfur atoms, where the nitrogen and sulfur heteroatoms may optionally be oxidized and the nitrogen heteroatoms may optionally be quaternized. (The term “heteroarylium” refers to a heteroaryl group bearing a quaternary nitrogen atom and thus a positive charge.) The heterocyclic group may be attached to the remainder of the molecule at any heteroatom or carbon atom of the ring or ring system. Exemplary monocyclic heterocyclic groups include azetidinyl, pyrrolidinyl, pyrrolyl, pyrazolyl, oxetanyl, pyrazolinyl, imidazolyl, imidazolinyl, imidazolidinyl, oxazolyl, oxazolidinyl, isoxazolinyl, isoxazolyl, thiazolyl, thiadiazolyl, thiazolidinyl, isothiazolyl, isothiazolidinyl, furyl, tetrahydrofuryl, thienyl, oxadiazolyl, piperidinyl, piperazinyl, 2-oxopiperazinyl, 2-oxopiperidinyl, 2-oxopyrrolodinyl, 2-oxoazepinyl, azepinyl, hexahydrodiazepinyl, 4-piperidonyl, pyridyl, pyrazinyl, pyrimidinyl, pyridazinyl, triazinyl, triazolyl, tetrazolyl, tetrahydropyranyl, morpholinyl, thiamorpholinyl, thiamorpholinyl sulfoxide, thiamorpholinyl sulfone, 1,3-dioxolane and tetrahydro-1,1-dioxothienyl, and the like. Exemplary bicyclic heterocyclic groups include indolyl, isoindolyl, benzothiazolyl, benzoxazolyl, benzoxadiazolyl, benzothienyl, quinuclidinyl, quinolinyl, tetrahydroisoquinolinyl, isoquinolinyl, benzimidazolyl, benzopyranyl, indolizinyl, benzofuryl, benzofurazanyl, chromonyl, coumarinyl, benzopyranyl, cinnolinyl, quinoxalinyl, indazolyl, pyrrolopyridyl, furopyridinyl (such as furo[2,3-c]pyridinyl, furo[3,2-b]pyridinyl] or furo[2,3-b]pyridinyl), dihydroisoindolyl, dihydroquinazolinyl (such as 3,4-dihydro-4-oxo-quinazolinyl), triazinylazepinyl, tetrahydroquinolinyl and the like. Exemplary tricyclic heterocyclic groups include carbazolyl, benzidolyl, phenanthrolinyl, acridinyl, phenanthridinyl, xanthenyl and the like.

“Substituted heterocycle” and “substituted heterocyclic” (such as “substituted heteroaryl”) refer to heterocycle or heterocyclic groups substituted with one or more substituents, preferably 1 to 4 substituents, at any available point of attachment. Exemplary substituents include, but are not limited to, cycloalkyl or substituted cycloalkyl, cycloalkenyl or substituted cycloalkenyl, nitro, oxo (i.e., ═O), cyano, alkyl or substituted alkyl, as well as those groups recited above as exemplary alkyl substituents. Exemplary substituents also include spiro-attached or fused cyclic substituents at any available point or points of attachment, especially spiro-attached cycloalkyl, spiro-attached cycloalkenyl, spiro-attached heterocycle (excluding heteroaryl), fused cycloalkyl, fused cycloalkenyl, fused heterocycle, or fused aryl, where the aforementioned cycloalkyl, cycloalkenyl, heterocycle and aryl substituents can themselves be optionally substituted. Exemplary substituents include but are not limited to one or more of the following groups: hydrogen, alkyl, haloalkyl, halogen (e.g., a single halogen substituent or multiple halo substitutents forming, in the latter case, groups such as CF₃ or an alkyl group bearing Cl₃), cyano, nitro, cycloalkyl, alkenyl, cycloalkenyl, alkynyl, heterocycle, aryl, ORₐ, SRₐ, S(═O)Rₑ, S(═O)₂Rₑ, P(═O)₂Rₑ, S(═O)₂ORₑ, P(═O)₂ORₑ, NR_{b}R_{c}, NR_{b}S(═O)₂Rₑ, NR_{b}P(═O)₂Rₑ, S(═O)₂NR_{b}R_{c}, P(═O)₂NR_{b}R_{c}, C(═O)ORₑ, C(═O)Rₐ, C(═O)NR_{b}R_{c}, OC(═O)Rₐ, OC(═O)NR_{b}R_{c}, NR_{b}C(═O)ORₑ, NR_{d}C(═O)NR_{b}R_{c}, NR_{d}S(═O)₂NR_{b}R_{c}, NR_{d}P(═O)₂NR_{b}R_{c}, NR_{b}C(═O)Rₐ, or NR_{b}P(═O)₂Rₑ, wherein Rₐ is hydrogen, alkyl, cycloalkyl, alkenyl, cycloalkenyl, alkynyl, heterocycle, or aryl; R_{b}, R_{c} and R_{d} are independently hydrogen, alkyl, cycloalkyl, heterocycle, aryl, or said R_{b} and R_{c} together with the N to which they are bonded optionally form a heterocycle; and Rₑ is alkyl, cycloalkyl, alkenyl, cycloalkenyl, alkynyl, heterocycle, or aryl. In the aforementioned exemplary substitutents, groups such as alkyl, cycloalkyl, alkenyl, alkynyl, cycloalkenyl, heterocycle and aryl can themselves be optionally substituted. The term “quaternary nitrogen” refers to a tetravalent positively charged nitrogen atom including, for example, the positively charged nitrogen in a tetraalkylammonium group (e.g., tetramethylammonium, N-methylpyridinium), the positively charged nitrogen in protonated ammonium species (e.g., trimethyl-hydroammonium, N-hydropyridinium), the positively charged nitrogen in amine N-oxides (e.g., N-methyl-morpholine-N-oxide, pyridine-N-oxide), and the positively charged nitrogen in an N-amino-ammonium group (e.g., N-aminopyridinium).

The terms “halogen” or “halo” refer to chlorine, bromine, fluorine or iodine.

The term “carbocyclic” refers to aromatic or non-aromatic 3 to 7 membered monocyclic and 7 to 11 membered bicyclic groups, in which all atoms of the ring or rings are carbon atoms. “Substituted carbocyclic” refers to a carbocyclic group substituted with one or more substituents, preferably 1 to 4 substituents, at any available point of attachment. Exemplary substituents include, but are not limited to, nitro, cyano, ORₐ, wherein Rₐ is as defined hereinabove, as well as those groups recited above as exemplary cycloalkyl substituents.

When a functional group is termed “protected”, this means that the group is in modified form to mitigate, especially preclude, undesired side reactions at the protected site. Suitable protecting groups for the methods and compounds described herein include, without limitation, those described in standard textbooks, such as Greene, T. W. et al., Protective Groups in Organic Synthesis, Wiley, N.Y. (1999).

Unless otherwise indicated, any heteroatom with unsatisfied valences is assumed to have hydrogen atoms sufficient to satisfy the valences.

The compounds of formula I form salts which are also within the scope of this invention. Reference to a compound of the formula I herein is understood to include reference to salts thereof, unless otherwise indicated. The term “salt(s)”, as employed herein, denotes acidic and/or basic salts formed with inorganic and/or organic acids and bases. In addition, when a compound of formula I contains both a basic moiety, such as but not limited to a pyridine or imidazole, and an acidic moiety such as but not limited to a carboxylic acid, zwitterions (“inner salts”) may be formed and are included within the term “salt(s)” as used herein. Pharmaceutically acceptable (i.e., non-toxic, physiologically acceptable) salts are preferred, although other salts are also useful, e.g., in isolation or purification steps which may be employed during preparation. Salts of the compounds of the formula I may be formed, for example, by reacting a compound I with an amount of acid or base, such as an equivalent amount, in a medium such as one in which the salt precipitates or in an aqueous medium followed by lyophilization.

The compounds of formula I which contain a basic moiety, such as but not limited to an amine or a pyridine or imidazole ring, may form salts with a variety of organic and inorganic acids. Exemplary acid addition salts include acetates (such as those formed with acetic acid or trihaloacetic acid, for example, trifluoroacetic acid), adipates, alginates, ascorbates, aspartates, benzoates, benzenesulfonates, bisulfates, borates, butyrates, citrates, camphorates, camphorsulfonates, cyclopentanepropionates, digluconates, dodecylsulfates, ethanesulfonates, fumarates, glucoheptanoates, glycerophosphates, hemisulfates, heptanoates, hexanoates, hydrochlorides, hydrobromides, hydroiodides, hydroxyethanesulfonates (e.g., 2-hydroxyethanesulfonates), lactates, maleates, methanesulfonates, naphthalenesulfonates (e.g., 2-naphthalenesulfonates), nicotinates, nitrates, oxalates, pectinates, persulfates, phenylpropionates (e.g., 3-phenylpropionates), phosphates, picrates, pivalates, propionates, salicylates, succinates, sulfates (such as those formed with sulfuric acid), sulfonates, tartrates, thiocyanates, toluenesulfonates such as tosylates, undecanoates, and the like.

The compounds of formula I which contain an acidic moiety, such but not limited to a carboxylic acid, may form salts with a variety of organic and inorganic bases. Exemplary basic salts include ammonium salts, alkali metal salts such as sodium, lithium and potassium salts, alkaline earth metal salts such as calcium and magnesium salts, salts with organic bases (for example, organic amines) such as benzathines, dicyclohexylamines, hydrabamines (formed with N,N-bis(dehydroabietyl)ethylenediamine), N-methyl-D-glucamines, N-methyl-D-glycamides, t-butyl amines, and salts with amino acids such as arginine, lysine and the like. Basic nitrogen-containing groups may be quaternized with agents such as lower alkyl halides (e.g. methyl, ethyl, propyl, and butyl chlorides, bromides and iodides), dialkyl sulfates (e.g. dimethyl, diethyl, dibutyl, and diamyl sulfates), long chain halides (e.g. decyl, lauryl, myristyl and stearyl chlorides, bromides and iodides), aralkyl halides (e.g. benzyl and phenethyl bromides), and others.

Prodrugs and solvates of the compounds of the invention are also contemplated herein. The term “prodrug” as employed herein denotes a compound that, upon administration to a subject, undergoes chemical conversion by metabolic or chemical processes to yield a compound of the formula I, or a salt and/or solvate thereof. Solvates of the compounds of formula I include, for example, hydrates.

Compounds of the formula I, and salts thereof, may exist in their tautomeric form (for example, as an amide or imino ether). All such tautomeric forms are contemplated herein as part of the present invention.

All stereoisomers of the present compounds (for example, those which may exist due to asymmetric carbons on various substituents), including enantiomeric forms and diastereomeric forms, are contemplated within the scope of this invention. Individual stereoisomers of the compounds of the invention may, for example, be substantially free of other isomers (e.g., as a pure or substantially pure optical isomer having a specified activity), or may be admixed, for example, as racemates or with all other, or other selected, stereoisomers. The chiral centers of the present invention may have the S or R configuration as defined by the IUPAC 1974 Recommendations. The racemic forms can be resolved by physical methods, such as, for example, fractional crystallization, separation or crystallization of diastereomeric derivatives or separation by chiral column chromatography. The individual optical isomers can be obtained from the racemates by any suitable method, including without limitation, conventional methods, such as, for example, salt formation with an optically active acid followed by crystallization.

Compounds of the formula I are, subsequent to their preparation, may be isolated and purified to obtain a composition containing an amount by weight equal to or greater than 99% formula I compound (“substantially pure” compound I), which is then used or formulated as described herein. Such “substantially pure” compounds of the formula I are also contemplated herein as part of the present invention.

All configurational isomers of the compounds of the present invention are contemplated, either in admixture or in pure or substantially pure form. The definition of compounds of the present invention embraces both cis (Z) and trans (E) alkene isomers, as well as cis and trans isomers of cyclic hydrocarbon or heterocyclic rings.

“Therapeutically effective amount” is intended to include an amount of a compound of the present invention alone or an amount of the combination of compounds claimed or an amount of a compound of the present invention in combination with other active ingredients effective to inhibit CARM-1 or effective to treat or prevent inflammatory disorders.

As used herein, “treating” or “treatment” cover the treatment of a disease-state in a mammal, particularly in a human, and include: (a) preventing the disease-state from occurring in a mammal, in particular, when such mammal is predisposed to the disease-state but has not yet been diagnosed as having it; (b) inhibiting the disease-state, i.e., arresting it development; and/or (c) relieving the disease-state, i.e., causing regression of the disease state.

Throughout the specifications, groups and substituents thereof may be chosen to provide stable moieties and compounds.

### Biological Assay

The pharmacological properties of the compounds of this invention may be confirmed by a number of pharmacological assays. The exemplified pharmacological assays which follow have been carried out with the compounds according to the invention and/or their pharmaceutically acceptable salts.

A methylation based filter assay was devised to test compounds that specifically inhibited CARM 1 dependent methylation. Human full length CARM1 purified from baculovirus infected insect cells was used as the source for enzyme. Histone H3 (Roche Applied Science) was used as the preferred substrate for the assay since the methylation of several amino acids of Histone H3 by CARM1 has been well documented. Tritiated S-Adenosyl-L-Methionine (SAM) (Amersham Pharmacia Biotech) was used as a cofactor since CARM1 exhibits an absolute requirement for SAM for its catalytic activity. Methylation reactions were performed for 75-90 minutes at room temperature using enzyme (CARM-1), substrate (Histone H3) and cofactor (SAM) in the presence of methylation buffer (20 mM Tris.HCl pH 8.0, 200 mM NaCl, 0.4 mM EDTA) and in the presence or absence of compound. Reactions were terminated using TCA and precipitated with BSA overnight. They were harvested the next morning by passing the reactions through a filter and the filters washed. The signal on the filters was read in a Top Count after addition of MicroScint-20.

Protocol for Methylation Based Filter Assay

I. Reaction Mixtures and Solutions:

10× Methyl transferase Buffer (MTB):

20 mM Tris HCl, pH 8.0

200 mM NaCl

0.4 mM EDTA
3× Test Compound Solution (15 μl per well):
Test compounds are diluted to 3 fold of final concentration with 1×MTB. For example, if the final concentration of test compound is designed as 30 μM, then 90 μM compound solution should be made at this step.
Diluted DMSO Solution (15 μl per well):
100% DMSO was diluted with 1×MTB to the same concentration as the compound solution. For example, if the 3× concentrate test compound solution contains 3% DMSO then 3% DMSO should be used for control and blank wells.
CARM1 Enzyme Mixture (15 μl per well):
   Pre-determined amount of CARM1 Enzyme
   0.03 μl 1M DTT
      0.03 μl 100 mg/ml BSA
         1.5 μl 10×MTB
      13.39 μl H₂O

Histone H3/[³H] SAM Mixture (15 μl per well):
0.8 μl 1 mg/ml Histone H3
   0.2 μl 1 mCi/ml [3H] SAM (65-80 Ci/mmol)
1.5 μl 10×MTB
   12.5 μl H₂O
Stop Solution (45 μl per well):
   20% TCA
   100 mM Sodium pyrophosphate
      II. Reaction Steps:
A plate layout, which contains blank (no enzyme), positive control (no compound) and test compound wells was created
The following reaction mixtures and solutions were added to wells taking care to avoid cross contamination:
   15 μl 1×MTB to each of blank wells
   15 μl diluted DMSO solution to each of blank and positive control wells
   15 μl 3× test compound solution to each of test compound wells
   15 μl CARM1 Mixture to each of compound and positive control wells
   15 μl Histone H3/[3H] SAM mixture to all wells
Plates were incubated for 60 minutes at room temperature (22-24° C.)
45 μl of stop solution was added to all wells
Plates were left overnight at 4° C.
The next day reaction mixes were harvested to filter plate with a Unifilter harvester
The filter plates were washed twice with 10% TCA and 4 times with H₂O
Air dry filter plate
30 μl Microscint-20 was added to each well and plates were covered with Top-seal
Plates were read in the Top Count and the data was analyzed to generate the IC₅₀ values.
   III. The Final Concentration of Components (in 60 μl total reaction volume):
CARM1 Enzyme=as pre-determined
   Histone H3=0.8 μg (1.16 μM)
      [3H] SAM=0.2 μCi (56 nM)
         DMSO=1%

Compounds of the present invention have activity in the above described assay. Compounds of the present invention described herein have an IC₅₀ of less than 10 μM.

Protocol for 3H Thymidine Incorporation Assay for IC₅₀ Evaluation

Inhibition of tumor cell proliferation upon treatment with compounds was monitored using the 3H thymidine incorporation assay. Cells of appropriate density were plated in 96-well plates and compounds added on the same day. Compound treatment was continued for either 3 or 6 days and dose response curves were determined as described below.

Materials and Reagents RPMI media
Fetal Bovine Serum
Trypsin-EDTA
DMSO
96 Deep-well plate, 24/box
[6-3H]Thymidine, 5 mCi
Unifilter-96, GF-B
Microscient PS
TopSeal-A:96-Well Microplates
   Assay Media:
   RPMI media+2.5% FBS
   Compound dilution:

A 96 deep-well plate was used for compound dilution. Compounds were diluted 1:3 serially. A 10-point dilution was performed starting at a concentration of 40 μM (2×). Assay media in columns 2-10 contained 0.4% DMSO (2×).

An example is shown below:

| | 1 | 2 | 3 | 4 | 5 | 6 | 7 | 8 | 9 | 10 |
|---|---|---|---|---|---|---|---|---|---|---|
| Conc. | 40 | 13.333 | 4.444 | 1.482 | 0.494 | 0.165 | 0.055 | 0.018 | 0.06 | 0.02 |
| μM(2X) | | | | | | | | | | |
| Assay | 0.9 | 0.6 | 0.6 | 0.6 | 0.6 | 0.6 | 0.6 | 0.6 | 0.6 | 0.6 |
| media (ml) | | | | | | | | | | |
| 10 μM | 3.6 | | | | | | | | | |
| Compd | | | | | | | | | | |
| (μl) | | | | | | | | | | |

Mix well and sequential dilution

| | | | | | | | | | |
|---|---|---|---|---|---|---|---|---|---|
| Transfer(ml): | 0.3 | 0.3 | 0.3 | 0.3 | 0.3 | 0.3 | 0.3 | 0.3 | 0.3 |

Preparation of cell suspension solution:

2×103-4×104 cells/ml (depending on cell type) of cell suspension was prepared in the assay media. Following is an example:

| | |
|---|---|
| Day 3 | Day 6 |

| Cell line | Cells/well | Cells/ml | Cells/well | Cells/ml |
|---|---|---|---|---|
| A549 | 500 | 1.0 × 10⁴ | 100 | 2 × 10³ |
| | 600 | 1.2 × 10⁴ | 200 | 4 × 10³ |
| | 1000 | 2 × 10⁴ | 500 | 1 × 10⁴ |
| | 2000 | 4 × 10⁴ | 1000 | 2 × 10⁴ |

Plate cells:
4 compounds were tested per plate.

50 μl of 2× compounds and 50 μl of cell suspension were added per well of a 96-well plate.

| | 1 | 2 | 3 | 4 | 5 | 6 | 7 | 8 | 9 | 10 | 11 | 12 |
|---|---|---|---|---|---|---|---|---|---|---|---|---|
| Conc. | 20 | 6.667 | 2.222 | 0.74 | 0.245 | 0.082 | 0.027 | 0.009 | 0.003 | 0.001 | 0 | |
| μM | | | | | | | | | | | | |

| | |
|---|---|
| A | Compound 1 |
| B | |
| C | Compound 2 |
| D | |
| E | Compound 3 |
| F | |
| G | Compound 4 |
| H | |

Note:
Column 11 was the control. 50 ul of assay media containing 0.4% DMSO and 50 μl of cell suspension were added per well.

On day 3 and day 6:
Addition of [³H] thymidine media:
   Dilution of [³H] thymidine solution: 44 μl of thymidine stock liquid was mixed with 956 μl of assay media for a total of 1 ml. 10 μl of diluted thymidine solution was added into each well (final concentration of thymidine is 4 μCi/well).
Harvest cells:
Media from the plates was discarded and 75 μl of 0.25% trypsin-EDTA was added to each well. Plates were incubated at 37° C. for 15 min. Cells were then harvested with Unifilter-96 GF-B plate by using the harvester.
Air dry plate: Plates were dried overnight.

Data analysis:
The bottom of the plate was sealed with sealing paper.
50 μl of Microscint™ PS was added to each well and the top of the plate was sealed with topseal.
The plate was read on a Topcon instrument and the data was analyzed to generate the IC₅₀ value.

The same protocol was used for the other cancer cell lines.

The compounds of the present invention have been tested in the following cell lines MDA-231 (breast); MDA-453 (breast); MDA-468 (breast); HS-578T (breast); DU-4475 (breast); BT-549 (breast); MCF-7 (breast;) K562 (leukemia); MolT4 (leukemia;) CCRF-CEM (leukemia); OCZ-CY19 (lymphoma); SK-Mel5 (melanoma); SK-Mel28 (melanoma); A549 (lung;) LX1 (lung); H23 (lung); H226 (lung); H522 (lung); H661 (lung); A375 (lung); and MSTO-211H (lung). Additionally, compounds of the present invention may be tested in the following cell lines: SW480 (colon); HCT116 (colon); PC3 (prostate); and LnCaP (prostate).

Compounds of the present invention have activity in the above described assay. Compounds of the present invention described herein have an IC₅₀ of less than 10 μM.

In another embodiment of the invention, a method is provided for treating a proliferative disease via modulation of CARM-1 (PRMT-4) by administering to a patient in need of such treatment an effective amount of a compound of formula I, as defined above, in combination (simultaneously or sequentially) with at least one other anti-cancer agent. In a preferred embodiment, the proliferative disease is cancer.

The invention further provides pharmaceutical compositions comprising compounds having formula I together with a pharmaceutically acceptable carrier.

More specifically, the compounds of Formulas I are useful in the treatment of a variety of cancers, including, but not limited to, the following:

a) carcinoma, including that of the bladder, breast, colon, kidney, liver, lung, including small cell lung cancer, esophagus, gall bladder, ovary, pancreas, stomach, cervix, thyroid, prostate, and skin, including squamous cell carcinoma;

b) hematopoietic tumors of lymphoid lineage, including leukemia, acute lymphocytic leukemia, acute lymphoblastic leukemia, B-cell lymphoma, T-cell lymphoma, Hodgkin's lymphoma, non-Hodgkins lymphoma, hairy cell lymphoma and Burkett's lymphoma;

c) hematopoietic tumors of myeloid lineage, including acute and chronic myelogenous leukemias, myelodysplastic syndrome and promyelocytic leukemia;

d) tumors of mesenchymal origin, including fibrosarcoma and rhabdomyosarcoma;

e) tumors of the central and peripheral nervous system, including astrocytoma, neuroblastoma, glioma and schwannomas; and

f) other tumors, including melanoma, seminoma, teratocarcinoma, osteosarcoma, xenoderoma pigmentosum, keratoctanthoma, thyroid follicular cancer and Kaposi's sarcoma.

The compounds of Formula I are useful in the treatment of breast cancer, leukemia, melanoma, lung cancer, colon cancer and prostate cancer.

The compounds of the present invention may be employed alone or in combination with each other and/or other suitable therapeutic agents useful in the treatment of cancer such as PTK inhibitors, antiinflammatories, antiproliferatives, chemotherapeutic agents, immunosuppressants, anticancer agents and cytotoxic agents.

Exemplary classes of anti-cancer agents and cytotoxic agents include, but are not limited to: alkylating agents, such as nitrogen mustards, alkyl sulfonates, nitrosoureas, ethylenimines, and triazenes; antimetabolites, such as folate antagonists, purine analogues, and pyrimidine analogues; antibiotics, such as anthracyclines (e.g., daunorubicin, doxorubicin), cytarabine (ara-C; Cytosar-U®); 6-thioguanine (Tabloid®), mitoxantrone (Novantrone®) and etoposide (VePesid®), amsacrine (AMSA), and all-trans retinoic acid (ATRA), bleomycins, mitomycin, dactinomycin, and plicamycin; enzymes, such as L-asparaginase; farnesyl-protein transferase inhibitors; hormonal agents, such as glucocorticoids, estrogens/antiestrogens, androgens/antiandrogens, progestins, and luteinizing hormone-releasing hormone anatagonists, octreotide acetate; microtubule-disruptor agents, such as ecteinascidins or their analogs and derivatives; microtubule-stabilizing agents such as paclitaxel (Taxol®), docetaxel (Taxotere®), and epothilones A-F or their analogs or derivatives; plant-derived products, such as vinca alkaloids, epipodophyllotoxins, taxanes; and topoisomerase inhibitors; prenyl-protein transferase inhibitors; and miscellaneous agents such as, hydroxyurea, procarbazine, mitotane, hexamethylmelamine, platinum coordination complexes such as cisplatin and carboplatin; and other agents used as anti-cancer and cytotoxic agents such as biological response modifiers, growth factors; immune modulators, and monoclonal antibodies. The compounds of the invention may also be used in conjunction with radiation therapy.

Representative examples of these classes of anti-cancer and cytotoxic agents include, but are not limited to, mechlorethamine hydrochloride, cyclophosphamide, chlorambucil, melphalan, ifosfamide, busulfan, carmustin, lomustine, semustine, streptozocin, thiotepa, dacarbazine, methotrexate, thioguanine, mercaptopurine, fludarabine, pentastatin, cladribin, cytarabine, fluorouracil, doxorubicin hydrochloride, daunorubicin, idarubicin, bleomycin sulfate, mitomycin C, actinomycin D, safracins, saframycins, quinocarcins, discodermolides, vincristine, vinblastine, vinorelbine tartrate, etoposide, teniposide, paclitaxel, tamoxifen, estramustine, estramustine phosphate sodium, flutamide, buserelin, leuprolide, pteridines, diyneses, levamisole, aflacon, interferon, interleukins, aldesleukin, filgrastim, sargramostim, rituximab, BCG, tretinoin, irinotecan hydrochloride, betamethosone, gemcitabine hydrochloride, altretamine, and topoteca and any analogs or derivatives thereof.

Other members of these classes include, but are not limited to paclitaxel, cisplatin, carboplatin, doxorubicin, carminomycin, daunorubicin, aminopterin, methotrexate, methopterin, mitomycin C, ecteinascidin 743, porfiromycin, 5-fluorouracil, 6-mercaptopurine, gemcitabine, cytosine arabinoside, podophyllotoxin or podophyllotoxin derivatives such as etoposide, etoposide phosphate or teniposide, melphalan, vinblastine, vincristine, leurosidine, vindesine, and leurosine.

In a combination, the other component(s) of such conjoint treatment in addition to the antiproliferative treatment defined herein before may be: surgery, radiotherapy or chemotherapy. Such chemotherapy may cover three main categories of therapeutic agent:
(i) antiangiogenic agents that work by different mechanisms from those defined hereinbefore (for example, linomide, inhibitors of integrin αvβ3 function, angiostatin, razoxane);
(ii) cytostatic agents such as antiestrogens (for example, tamoxifen, toremifene, raloxifene, droloxifene, iodoxifene), progestogens (for example megestrol acetate), aromatase inhibitors (for example, anastrozole, letrozole, borazole, exemestane), antihormones, antiprogestogens, antiandrogens (for example, flutamide, nilutamide, bicalutamide, cyproterone acetate), LHRH agonists and antagonists (for example, gosereline acetate, leuprolide), inhibitors of testosterone 5α-dihydroreductase (for example, finasteride), farnesyltransferase inhibitors, anti-invasion agents (for example, metalloproteinase inhibitors like marimastat and inhibitors of urokinase plasminogen activator receptor function) and inhibitors of growth factor function, (such growth factors include for example, EGF, FGF, platelet derived growth factor and hepatocyte growth factor such inhibitors include growth factor antibodies, growth factor receptor antibodies such as Avastin® (bevacizumab) and Erbitux® (cetuximab); tyrosine kinase inhibitors and serine/threonine kinase inhibitors such as Gleevec® (imatinib mesylate)); and
(iii) antiproliferative/antineoplastic drugs and combinations thereof, as used in medical oncology, such as antimetabolites (for example, antifolates like methotrexate, fluoropyrimidines like 5-fluorouracil, purine and adenosine analogues, cytosine arabinoside); Intercalating antitumour antibiotics (for example, anthracyclines like doxorubicin, daunomycin, epirubicin and idarubicin, mitomycin-C, dactinomycin, mithramycin); platinum derivatives (for example, cisplatin, carboplatin); alkylating agents (for example, nitrogen mustard, melphalan, chlorambucil, busulphan, cyclophosphamide, ifosfamide nitrosoureas, thiotepa; antimitotic agents (for example, vinca alkaloids like vincristine, vinblastine and vinflunine, and taxoids like Taxol® (paclitaxel), Taxotere® (docetaxel) and newer microbtubule agents such as epothilone analogs, discodermolide analogs, and eleutherobin analogs); topoisomerase inhibitors (for example, epipodophyllotoxins like etoposide and teniposide, amsacrine, topotecan, CPT-11); cell cycle inhibitors (for example, flavopyridols); biological response modifiers and proteasome inhibitors such as Velcade® (bortezomib).

The term “anticancer” agent includes any known agent that is useful for the treatment of cancer including the following: 17α-ethinylestradiol, diethylstilbestrol, testosterone, prednisone, fluoxymesterone, dromostanolone propionate, testolactone, megestrolacetate, methylprednisolone, methyl-testosterone, prednisolone, triamcinolone, chlorotrianisene, hydroxyprogesterone, aminoglutethimide, estramustine, medroxyprogesteroneacetate, leuprolide, flutamide, toremifene, Zoladex; matrix metalloproteinase inhibitors; VEGF inhibitors, such as anti-VEGF antibodies (Avastin) and small molecules such as ZD6474, AZD-2171, SU6668; Vatalanib, BAY-43-9006, SU11248, CP-547632, and CEP-7055; Her 1 and Her 2 inhibitors including anti-Her2 antibodies (Herceptin); EGFR inhibitors including gefitinib, erlotinib, ABX-EGF, EMD72000, 11F8, and cetuximab; Eg5 inhibitors, such as SB-715992, SB-743921, and MKI-833; pan Her inhibitors, such as canertinib, EKB-569, CI-1033, AEE-788, XL-647, mAb 2C4, and GW-572016; Src kinase inhibitors such as BMS-354825, AZD-0530, SKI-606, and AP-23464; Bcr-Abl inhibitors such as imatinib and AMN107; Casodex® (bicalutamide, Astra Zeneca), Tamoxifen; MEK-1 kinase inhibitors, MAPK kinase inhibitors, PI3 kinase inhibitors; Met inhibitors, Aurora kinase inhibitors, PDGF inhibitors; anti-angiogenic and antivascular agents which, by interrupting blood flow to solid tumors, render cancer cells quiescent by depriving them of nutrition; castration, which renders androgen dependent carcinomas non-proliferative; IGF1R inhibitors such as those disclosed in US2004/44203A1, inhibitors of non-receptor and receptor tyrosine kinases; inhibitors of integrin signaling; tubulin acting agents such as vinblastine, vincristine, vinorelbine, vinflunine, paclitaxel, docetaxel, 7-O-methylthiomethylpaclitaxel, 4-desacetyl-4-methylcarbonatepaclitaxel, 3′-tert-butyl-3′-N-tert-butyloxycarbonyl-4-deacetyl-3′-dephenyl-3′-N-debenzoyl-4-O-methoxycarbonyl-paclitaxel, C-4 methyl carbonate paclitaxel, epothilone A, epothilone B, epothilone C, epothilone D, desoxyepothilone A, desoxyepothilone B, ixabepilone, [1S-[1R*,3R*(E),7R*,10S*,11R*,12R*,16S*]]-3-[2-[2-(aminomethyl)-4-thiazolyl]-1-methylethenyl]-7,11-dihydroxy-8,8,10,12,16-pentamethyl-4-17-dioxabicyclo[14.1.0]-heptadecane-5,9-dione, and derivatives thereof; CDK inhibitors, antiproliferative cell cycle inhibitors, epidophyllotoxin, etoposide, VM-26; antineoplastic enzymes, e.g., topoisomerase I inhibitors, camptothecin, topotecan, SN-38; procarbazine; mitoxantrone; platinum coordination complexes such as cisplatin, carboplatin and oxaliplatin; biological response modifiers; growth inhibitors; antihormonal therapeutic agents; leucovorin; tegafur; antimetabolites such as purine antagonists (e.g. 6-thioguanine and 6-mercaptopurine; glutamine antagonists, e.g. DON (AT-125; d-oxo-norleucine); ribonucleotide reductase inhibitors; mTOR inhibitors; and haematopoietic growth factors.

Additional cytotoxic agents include, cyclophosphamide, doxorubicin, daunorubicin, mitoxanthrone, melphalan, hexamethyl melamine, thiotepa, cytarabin, idatrexate, trimetrexate, dacarbazine, L-asparaginase, bicalutamide, leuprolide, pyridobenzoindole derivatives, interferons, and interleukins.

In cases where it is desirable to render aberrantly proliferative cells quiescent in conjunction with or prior to treatment with the chemotherapeutic methods of the invention, hormones and steroids (including synthetic analogs): 17a-Ethinylestradiol, Diethylstilbestrol, Testosterone, Prednisone, Fluoxymesterone, Dromostanolone propionate, Testolactone, Megestrolacetate, Methylprednisolone, Methyl-testosterone, Prednisolone, Triamcinolone, hlorotrianisene, Hydroxyprogesterone, Aminoglutethimide, Estramustine, Medroxyprogesteroneacetate, Leuprolide, Flutamide, Toremifene, Zoladex can also be administered to the patient.

Also suitable for use in the combination chemotherapeutic methods of the invention are antiangiogenics such as matrix metalloproteinase inhibitors, and other VEGF inhibitors, such as anti-VEGF antibodies and small molecules such as ZD6474 and SU6668 are also included. Anti-Her2 antibodies from Genetech may also be utilized. A suitable EGFR inhibitor is EKB-569 (an irreversible inhibitor). Also included are Imclone antibody C225 immunospecific for the EGFR, and src inhibitors.

Also suitable for use as an antiproliferative cytostatic agent is Casodex™ which renders androgen-dependent carcinomas non-proliferative. Yet another example of a cytostatic agent is the antiestrogen Tamoxifen which inhibits the proliferation or growth of estrogen dependent breast cancer. Inhibitors of the transduction of cellular proliferative signals are cytostatic agents. Examples are epidermal growth factor inhibitors, Her-2 inhibitors, MEK-1 kinase inhibitors, MAPK kinase inhibitors, PI3 inhibitors, Src kinase inhibitors, and PDGF inhibitors.

As mentioned, certain anti-proliferative agents are anti-angiogenic and antivascular agents and, by interrupting blood flow to solid tumors, render cancer cells quiescent by depriving them of nutrition. Castration, which also renders androgen dependent carcinomas non-proliferative, may also be utilized. Starvation by means other than surgical disruption of blood flow is another example of a cytostatic agent. A particular class of antivascular cytostatic agents is the combretastatins. Other exemplary cytostatic agents include MET kinase inhibitors, MAP kinase inhibitors, inhibitors of non-receptor and receptor tyrosine kinases, inhibitors of integrin signaling, and inhibitors of insulin-like growth factor receptors.

Compounds of Formulas I as modulators of apoptosis, will be useful in the treatment of cancer (including but not limited to those types mentioned herein above), viral infections (including but not limited to herpevirus, poxvirus, Epstein-Barr virus, Sindbis virus and adenovirus), prevention of AIDS development in HIV-infected individuals, autoimmune diseases (including but not limited to systemic lupus, erythematosus, autoimmune mediated glomerulonephritis, rheumatoid arthritis, psoriasis, inflammatory bowel disease, and autoimmune diabetes mellitus), neurodegenerative disorders (including but not limited to Alzheimer's disease, AIDS-related dementia, Parkinson's disease, amyotrophic lateral sclerosis, retinitis pigmentosa, spinal muscular atrophy and cerebellar degeneration), myelodysplastic syndromes, aplastic anemia, ischemic injury associated with myocardial infarctions, stroke and reperfusion injury, arrhythmia, atherosclerosis, toxin-induced or alcohol related liver diseases, hematological diseases (including but not limited to chronic anemia and aplastic anemia), degenerative diseases of the musculoskeletal system (including but not limited to osteoporosis and arthritis) aspirin-sensitive rhinosinusitis, cystic fibrosis, multiple sclerosis, kidney diseases and cancer pain.

Compounds of Formulas I may modulate the level of cellular RNA and DNA synthesis. These agents would therefore be useful in the treatment of viral infections (including but not limited to HIV, human papilloma virus, herpesvirus, poxvirus, Epstein-Barr virus, Sindbis virus and adenovirus).

Compounds of Formulas I may be useful in the chemoprevention of cancer. Chemoprevention is defined as inhibiting the development of invasive cancer by either blocking the initiating mutagenic event or by blocking the progression of pre-malignant cells that have already suffered an insult or inhibiting tumor relapse.

Compounds of Formulas I may also be useful in inhibiting tumor angiogenesis and metastasis.

The compounds of this invention may also be useful in combination (administered together or sequentially) with known anti-cancer treatments such as radiation therapy or with cytostatic or cytotoxic agents, such as for example, but not limited to, DNA interactive agents; either naturally occurring or synthetic; hormonal agents, such as tamoxifen; other tyrosine kinase inhibitors such as Iressa and OSI-774; c-Kit inhibitors.

The pharmaceutical compositions containing the active ingredient may be in a form suitable for oral use, for example, as tablets, troches, lozenges, aqueous or oily suspensions, dispersible powders or granules, emulsions, hard or soft capsules, or syrups or elixirs. Compositions intended for oral use may be prepared according to any method known to the art for the manufacture of pharmaceutical compositions and such compositions may contain one or more agents selected from the group consisting of sweetening agents, flavoring agents, coloring agents and preserving agents in order to provide pharmaceutically elegant and palatable preparations. Tablets contain the active ingredient in admixture with non-toxic pharmaceutically acceptable excipients which are suitable for the manufacture of tablets. These excipients may be for example, inert diluents, such as calcium carbonate, sodium carbonate, lactose, calcium phosphate or sodium phosphate; granulating and disintegrating agents, for example, microcrystalline cellulose, sodium crosscarmellose, corn starch, or alginic acid; binding agents, for example starch, gelatin, polyvinyl-pyrrolidone or acacia, and lubricating agents, for example, magnesium stearate, stearic acid or talc. The tablets may be uncoated or they may be coated by known techniques to mask the unpleasant taste of the drug or delay disintegration and absorption in the gastrointestinal tract and thereby provide a sustained action over a longer period. For example, a water soluble taste masking material such as hydroxypropyl-methylcellulose or hydroxypropyl-cellulose, or a time delay material such as ethyl cellulose, cellulose acetate buryrate may be employed.

Formulations for oral use may also be presented as hard gelatin capsules wherein the active ingredient is mixed with an inert solid diluent, for example, calcium carbonate, calcium phosphate or kaolin, or as soft gelatin capsules wherein the active ingredient is mixed with water soluble carrier such as polyethyleneglycol or an oil medium, for example peanut oil, liquid paraffin, or olive oil.

Aqueous suspensions contain the active material in admixture with excipients suitable for the manufacture of aqueous suspensions. Such excipients are suspending agents, for example sodium carboxymethylcellulose, methylcellulose, hydroxypropylmethyl-cellulose, sodium alginate, polyvinyl-pyrrolidone, gum tragacanth and gum acacia; dispersing or wetting agents may be a naturally-occurring phosphatide, for example lecithin, or condensation products of an alkylene oxide with fatty acids, for example polyoxyethylene stearate, or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more preservatives, for example ethyl, or n-propyl p-hydroxybenzoate, one or more coloring agents, one or more flavoring agents, and one or more sweetening agents, such as sucrose, saccharin or aspartame.

Oily suspensions may be formulated by suspending the active ingredient in a vegetable oil, for example arachis oil, olive oil, sesame oil or coconut oil, or in mineral oil such as liquid paraffin. The oily suspensions may contain a thickening agent, for example beeswax, hard paraffin or cetyl alcohol. Sweetening agents such as those set forth above, and flavoring agents may be added to provide a palatable oral preparation. These compositions may be preserved by the addition of an anti-oxidant such as butylated hydroxyanisol or alpha-tocopherol.

Dispersible powders and granules suitable for preparation of an aqueous suspension by the addition of water provide the active ingredient in admixture with a dispersing or wetting agent, suspending agent and one or more preservatives. Suitable dispersing or wetting agents and suspending agents are exemplified by those already mentioned above. Additional excipients, for example sweetening, flavoring and coloring agents, may also be present. These compositions may be preserved by the addition of an anti-oxidant such as ascorbic acid.

The pharmaceutical compositions of the invention may also be in the form of an oil-in-water emulsions. The oily phase may be a vegetable oil, for example olive oil or arachis oil, or a mineral oil, for example liquid paraffin or mixtures of these. Suitable emulsifying agents may be naturally-occurring phosphatides, for example soy bean lecithin, and esters or partial esters derived from fatty acids and hexitol anhydrides, for example sorbitan monooleate, and condensation products of the said partial esters with ethylene oxide, for example polyoxyethylene sorbitan monooleate. The emulsions may also contain sweetening, flavoring agents, preservatives and antioxidants.

Syrups and elixirs may be formulated with sweetening agents, for example glycerol, propylene glycol, sorbitol or sucrose. Such formulations may also contain a demulcent, a preservative, flavoring and coloring agents and antioxidant.

The pharmaceutical compositions may be in the form of a sterile injectable aqueous solutions. Among the acceptable vehicles and solvents that may be employed are water, Ringer's solution and isotonic sodium chloride solution.

The sterile injectable preparation may also be a sterile injectable oil-in-water microemulsion where the active ingredient is dissolved in the oily phase. For example, the active ingredient may be first dissolved in a mixture of soybean oil and lecithin. The oil solution then introduced into a water and glycerol mixture and processed to form a microemulation.

The injectable solutions or microemulsions may be introduced into a patient's blood-stream by local bolus injection. Alternatively, it may be advantageous to administer the solution or microemulsion in such a way as to maintain a constant circulating concentration of the instant compound. In order to maintain such a constant concentration, a continuous intravenous delivery device may be utilized. An example of such a device is the Deltec CADD-PLUS™ model 5400 intravenous pump.

The pharmaceutical compositions may be in the form of a sterile injectable aqueous or oleagenous suspension for intramuscular and subcutaneous administration. This suspension may be formulated according to the known art using those suitable dispersing or wetting agents and suspending agents which have been mentioned above. The sterile injectable preparation may also be a sterile injectable solution or suspension in a non-toxic parenterally-acceptable diluent or solvent, for example as a solution in 1,3-butane diol. In addition, sterile, fixed oils are conventionally employed as a solvent or suspending medium. For this purpose any bland fixed oil may be employed including synthetic mono- or diglycerides. In addition, fatty acids such as oleic acid find use in the preparation of injectables.

Compounds of Formulas I may also be administered in the form of a suppositories for rectal administration of the drug. These compositions can be prepared by mixing the drug with a suitable non-irritating excipient which is solid at ordinary temperatures but liquid at the rectal temperature and will therefore melt in the rectum to release the drug. Such materials include cocoa butter, glycerinated gelatin, hydrogenated vegetable oils, mixtures of polyethylene glycols of various molecular weights and fatty acid esters of polyethylene glycol.

For topical use, creams, ointments, jellies, solutions or suspensions, etc., containing the compound of Formula I are employed. (For purposes of this application, topical application shall include mouth washes and gargles.)

The compounds for the present invention can be administered in intranasal form via topical use of suitable intranasal vehicles and delivery devices, or via transdermal routes, using those forms of transdermal skin patches well known to those of ordinary skill in the art. To be administered in the form of a transdermal delivery system, the dosage administration will, of course, be continuous rather than intermittent throughout the dosage regimen. Compounds of the present invention may also be delivered as a suppository employing bases such as cocoa butter, glycerinated gelatin, hydrogenated vegetable oils, mixtures of polyethylene glycols of various molecular weights and fatty acid esters of polyethylene glycol.

When a compound according to this invention is administered into a human subject, the daily dosage will normally be determined by the prescribing physician with the dosage generally varying according to the age, weight, sex and response of the individual patient, as well as the severity of the patient's symptoms.

If formulated as a fixed dose, such combination products employ the compounds of this invention within the dosage range described above and the other pharmaceutically active agent or treatment within its approved dosage range. Compounds of Formula I may also be administered sequentially with known anticancer or cytotoxic agents when a combination formulation is inappropriate. The invention is not limited in the sequence of administration; compounds of Formula I may be administered either prior to or after administration of the known anticancer or cytotoxic agent(s).

### Methods of Preparation

The compounds of the present invention may be prepared by methods such as those illustrated in the following schemes. Solvents, temperatures, pressures, and other reaction conditions may readily be selected by one of ordinary skill in the art. Starting materials are commercially available or readily prepared by one of ordinary skill in the art. These schemes are illustrative and are not meant to limit the possible techniques one skilled in the art may use to manufacture compounds disclosed herein. Different methods may be evident to those skilled in the art. Additionally, the various steps in the synthesis may be performed in an alternate sequence or order to give the desired compound(s). All documents cited herein are incorporated herein by reference in their entirety.

In general, the time taken to complete a reaction procedure will be judged by the person performing the procedure, preferably with the aid of information obtained by monitoring the reaction by methods such as HPLC or TLC. A reaction does not have to go to completion to be useful to this invention. The methods for the preparation of various heterocycles used to this invention can be found in standard organic reference books, for example, “Comprehensive Heterocyclic Chemistry, The Structure, Reactions, Synthesis and Uses, of Heterocyclic Compounds,” Katritzky, A. R., Rees, C. W. Eds Pergamon Press New York, First edition 1984, and “Comprehensive Heterocyclic Chemistry II, A Review of the Literature 1982-1995: The Structure, Reactions, Synthesis and Uses, of Heterocyclic Compounds,” Katritzky, A. R., Rees, C. W. and Scriven, E., F. Eds Pergamon Press New York, 1996.

Unless otherwise specified, the various substituents of the compounds are defined in the same manner as the formula I compound of the invention.

Compounds of Formula I wherein W is CONR₈R₁₀ where R₈ is alkyl, cycloalkyl, aryl or heteroaryl and R₁₀ is H or small alkyl and R₃ is H; could be made using a general reaction sequence as depicted in Scheme 1. The corresponding heterocycle (2H-Q-CO₂R; where R is small alkyl) could be either purchased commercially or synthesized according to the known procedures in the literature (Pinto et al, J Med Chem, 566-578 (2001)). The coupling of H-Q-CO₂R with the substituted benzonitrile (1) could be carried out using alkali metal or transition metal salt mediated reaction typically carried out in polar aprotic solvent at 25-200° C. Conversion of cyano to the amino (3) could be accomplished using chemical or catalytic reduction (R₂═H) or addition of appropriate alkylmetal reagent followed by reduction as described in Synth. Commun. 4067-4075, 28(21) 1998. The free amino group can be combined with protected alfa-amino acid using standard peptide coupling conditions, such as BOP-Cl, HBTU or DCC. The ester group R, can be converted into the corresponding acid derivative (6) using acid or basic hydrolysis. The acid can be combined with HNR₇R₈ using classical or modified peptide bond formation conditions or using Lewis acid condition and finally the protective group (PG) can be removed using appropriate deprotecting conditions.

Alternatively, the heterocycle containing CO₂R can be built upon the benzonitrile derivative 8 where L is N or C; using various known cyclization reactions reported in the literature (WO6020357 and Bioorg Med Chem Lett, 641 (2001)). This can be taken forward to the target compound using the conditions as reported in Scheme 1. 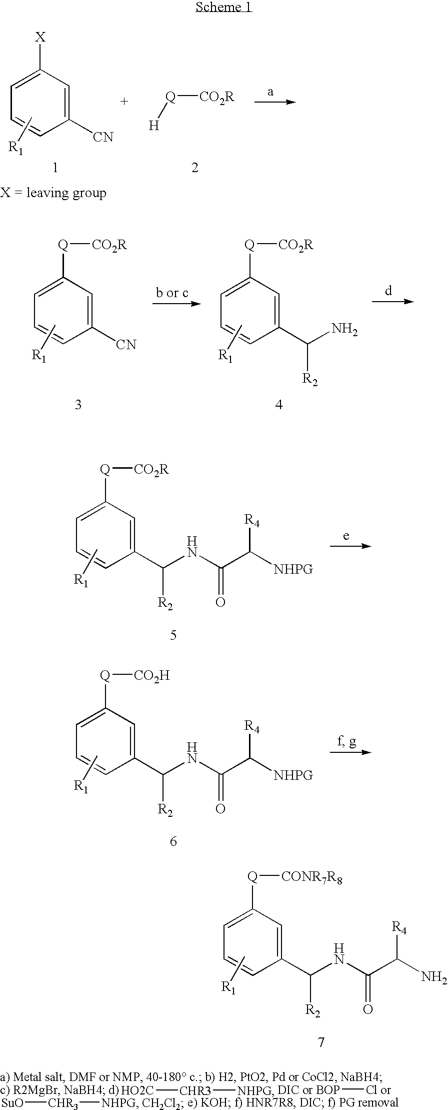 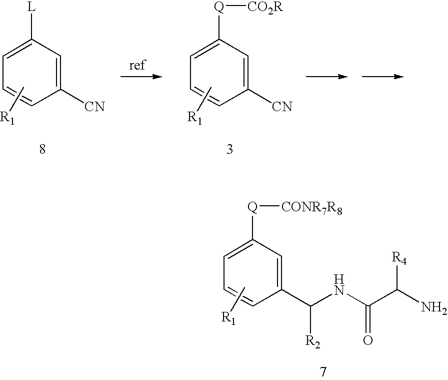
Likewise compounds of Formula I wherein W is COR₆R₈ where R₈ forms a heterocycloalkyl ring with Q and R₈ is aryl or heteroaryl; can be synthesized using a general reaction sequence as described Scheme 3. The required benzonitrile 9 can be synthesized using procedure as reported in WO2003049681. 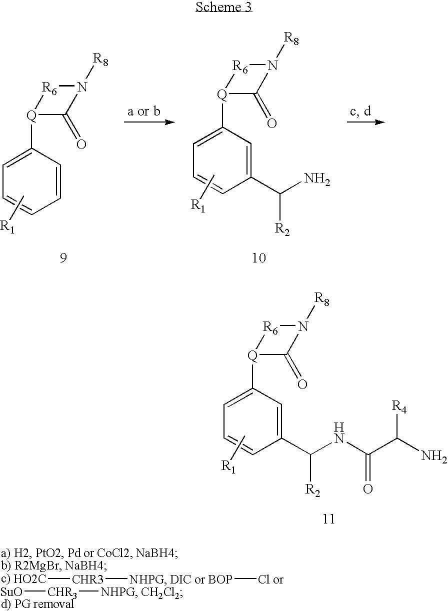
Compounds of Formula I wherein W is NR₉R₁₀ where R₉ is H and R₁₀ is COR₁₁; can be synthesized using a general reaction sequence as depicted in Scheme 4. Acid 6 can be converted into corresponding amine using Curtius rearrangement. To obtain amide derivatives (when R₁₁=aryl or heteroaryl or cycloalkyl) the amine can be coupled using acid chloride or fluoride or standard peptide coupling conditions. To furnish carbamate derivatives (when R₁₁═O-aryl, O-heteroaryl or O-cycloalkyl), the amine can be combined with corresponding halocarbonate or p-nitrophenyl carbonate. Corresponding urea (when R₁₁═NR₄R₅) derivatives can be made by coupling the amine with corresponding isocayanate or N,N-disubstituted carbamyl chloride derivative. The terminal amine functionality can be unmasked at the end using appropriate deprotection strategy.

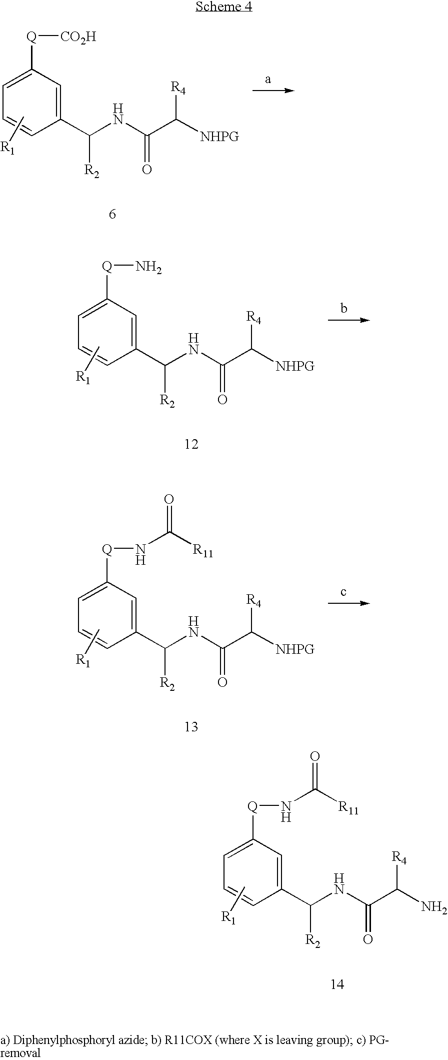

Compounds of formula I wherein W is a 1,3,4-oxidiazole is made by a general synthetic route described in Scheme 5. The acid 6 can be combined with acylhydrazide of type (R₁R₂CONHNH₂) using coupling reagents such as DIC or PyBrop to yield diacylhydrazide derivative 15. This can be cyclized to 16 with removal of water using reagents such as DIC or DCC or Burgess reagent at 60 to 150° C. Finally removal of protective group such as Boc can be accomplished with mineral or organic acid.

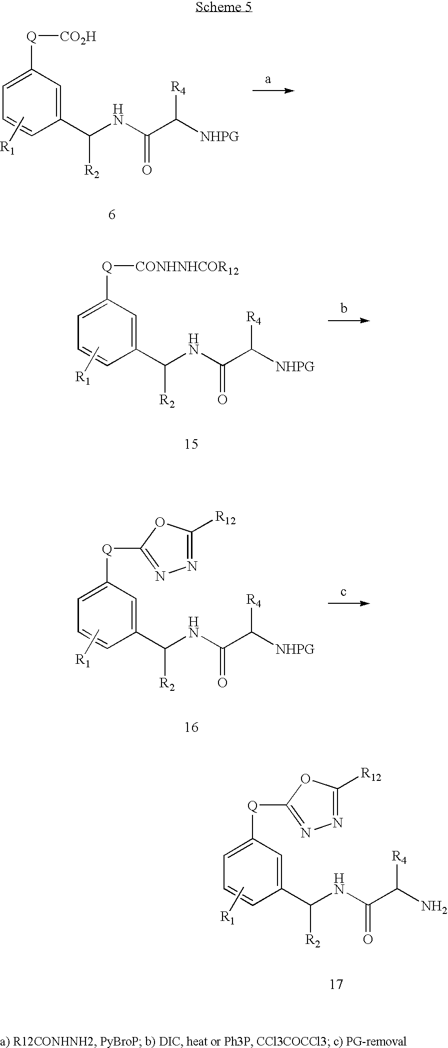

Similarly compounds of formula I wherein W is a 1,2,4-oxidiazole is made by a general synthetic route described in Scheme 6.

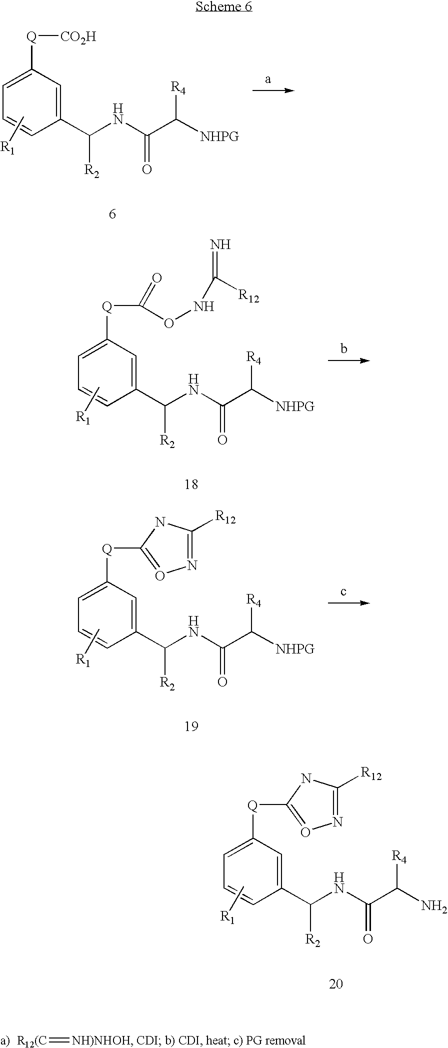

Compounds of formula I wherein W is an aryl fused oxazole or thiazole or imidazole is made by a general synthetic route described in Scheme 7.

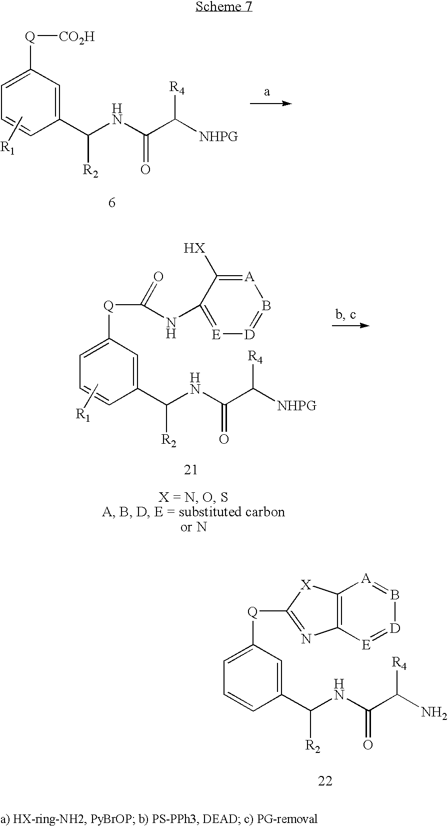

Compounds of formula I wherein W is 1,3,4-oxidiazole linked at 5 position through a nitrogen tether can be made by a general synthetic route described in Scheme 8. 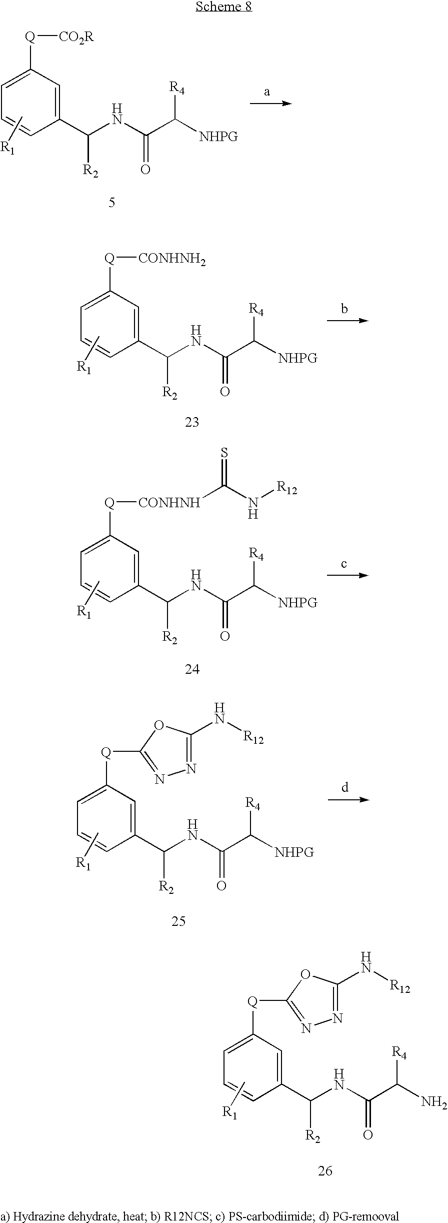
Similarly compounds of formula I wherein W is 1,3-oxazole can be made by a general synthetic route described in Scheme 9

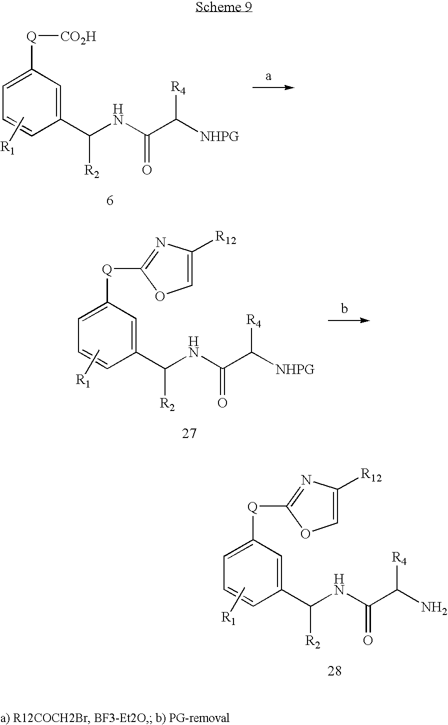

### EXAMPLES

### 1-(3-((2-aminopropanamido)methyl)phenyl)-N-benzyl-3-(trifluoromethyl)-1H-pyrazole-5-carboxamide (Example 1)

### Example 1

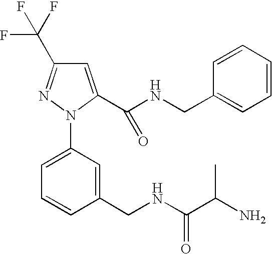

### Part A: Preparation of 1-(3-(aminomethyl)phenyl)-3-(trifluoromethyl)-1H-pyrazole-5-carboxylic acid

To a solution of 1-(3-cyanophenyl)-3-(trifluoromethyl)-1H-pyrazole-5-carboxylic acid (prepared using procedure reported in J Med Chem, 44, 566 (2001)) (1.40 g, 5.0 mmol) in THF (5 mL) and isopropanol (100 mL) was added 4M HCl in dioxane (5 mL) followed by Pd/C (10% wt, 500 mg) and PtO₂ (20 mg). The reaction was hydrogenated (55 psi H₂) for 20 hours and filtered through celite. The filtrate was concentrated in vacuo. The residue was dissolved in methanol and passed through SCX column (8×5 g), eluted first with methanol and then 2M NH₃ in methanol. The NH₃ elutants were combined and concentrated in vacuo. The residue was re-dissolved in methanol and triethylamine (1 mL) was added. This was concentrated in vacuo to remove any residual NH₄⁺, giving the title compound as a light yellow solid (1.00 g, 70%).

MS (ESI) m/z 286.07 (M+H)

### Part B: Preparation of 1-(3-((2-(tert butoxycarbonyl)propanamido)methyl)phenyl)-3-(trifluoromethyl)-1H-pyrazole-5-carboxylic acid

To a suspension of 2 (1.00 g, 3.5 mmol) and triethylamine (1.0 mL, 7.0 mmol) in CH₂Cl₂ (40 mL) was added Boc-Ala-OSu (1.10 g, 3.85 mmol). The reaction was stirred at room temperature for 2 hours, became homogeneous and was concentrated in vacuo. The residue was dissolved in methanol (4 mL). Water (40 mL) was added slowly, resulting in a cloudy mixture. To this was added 1.0 M aqueous HCl dropwise while stirring, until pH˜2. White precipitate formed and the mixture was filtered. The solid was washed with water, dissolved in methanol and concentrated in vacuo to give 3 as a light yellow glass (1.37 g). Another 86 mg of 3 was recovered from the filtrate when crystals formed after a few days, giving a total yield of 91% of the title compound. MS (ESI) m/z 457.17 (M+H)

NMR (CDCl3, δ, ppm): 10.0 (s, 1H), 8.1 (bs, 1H), 7.9 (s, 1H), 7.7 (d, 1H), 7.5 (d, 1H), 7.37 (dd, 1H), 4.52 (bs, 2H), 4.32 (m, 1H), 1.43 (s, 9H), 1.3 (d, 3H)

### Part C: Preparation of 1-(3-((2-aminopropanamido) methyl)phenyl)-N-benzyl-3-(trifluoromethyl)-1H-pyrazole-5-carboxamide trifluoroacetic acid salt (Example 1)

To a solution of HOAt (4.1 mg, 0.03 mmol), EDCI (5.8 mg, 0.03 mmol), and DIEA (0.017 mL, 0.10 mmol) in 1,2-dichloroethane (0.50 mL) was added 3 (9.1 mg, 0.02 mmol). The reaction was stirred at room temperature for 30 minutes before benzylamine (0.0055 mL, 0.05 mmol) was added. The reaction was stirred at room temperature for 20 hours and then heated at 65° C. for 5 hours. The reaction was concentrated in vacuo. To the residue was added CH₂Cl₂ (0.6 mL) followed by TFA (0.1 mL). The reaction was stirred at room temperature for 2 hours, concentrated in vacuo, azeotroped with methanol and purified by preparative HPLC to give Example 1 as a white solid (9.5 mg, 85%).

Conditions: Column—YMC ODS (20×50 mm)

Solvents—A—90% water—10% methanol—0.1% TFA
   B—10% water—90% methanol—0.1% TFA
Gradient—25% B to 100% B in 12 min
Retention time: 9 min

MS (ESI) m/z 446.18 (M+H)

NMR (CD3OD): 8.2 (s, 1H), 7.96 (s, 1H), 7.71 (d, 1H), 7.33 (m, 5H), 7.2 (m, 1H), 7.1 (d, 1H), 4.6 (s, 1H), 4.4 (s, 1H), 3.9 (m, 1H), 1.3 (d, 3H)

Preparation of Examples 2 to 85: The following examples were prepared using a method analogous to that used to prepare Example 1.

TABLE 1

| Example # | Structure | MS m/z |
|---|---|---|
| 2 | 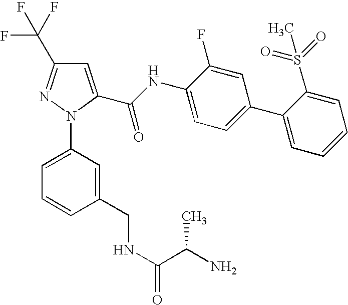 | 604.6 |
| 3 | 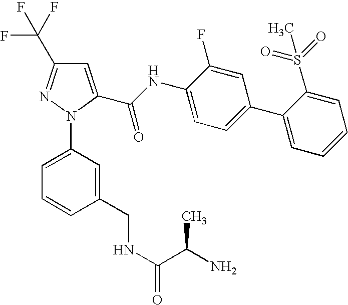 | 604.6 |
| 4 | 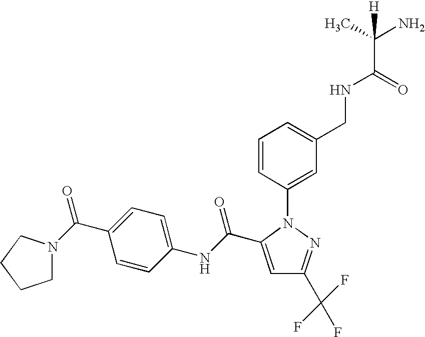 | 529.5 |
| 5 | 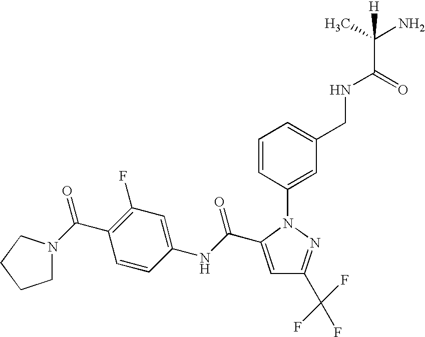 | 547.5 |
| 6 | 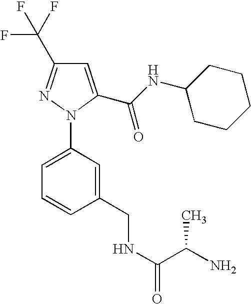 | 438.47 |
| 7 | 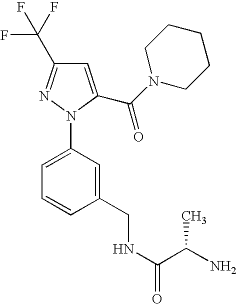 | 424.44 |
| 8 | 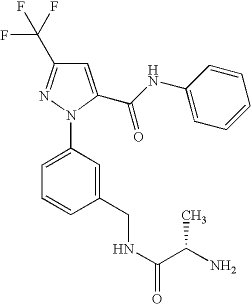 | 432.42 |
| 9 | 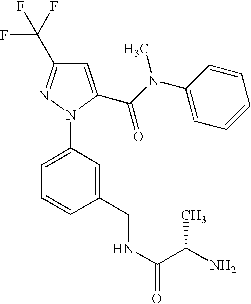 | 446.45 |
| 10 | 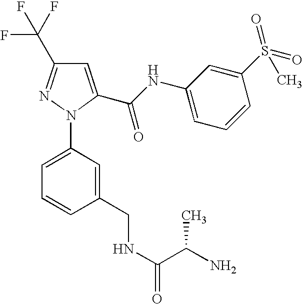 | 510.51 |
| 11 | 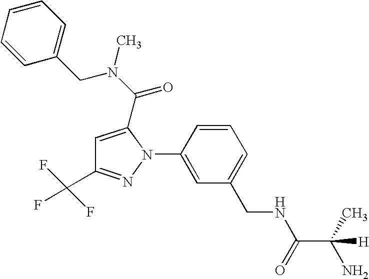 | 460.5 |
| 12 | 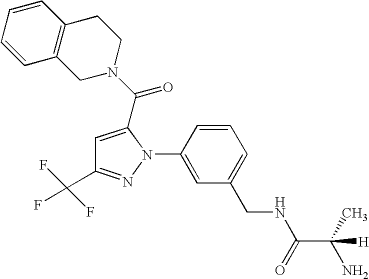 | 472.5 |
| 13 | 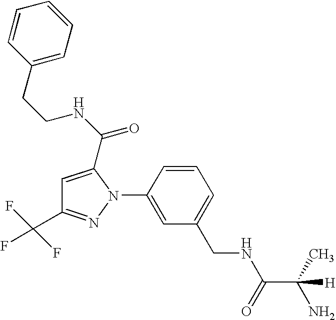 | 460.5 |
| 14 | 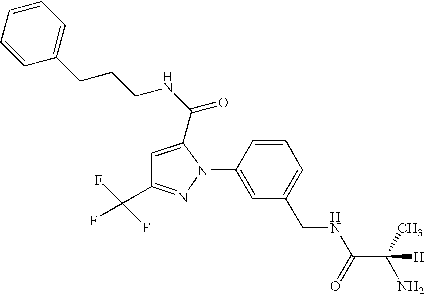 | 474.5 |
| 15 | 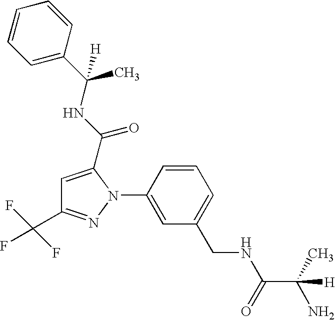 | 460.5 |
| 16 | 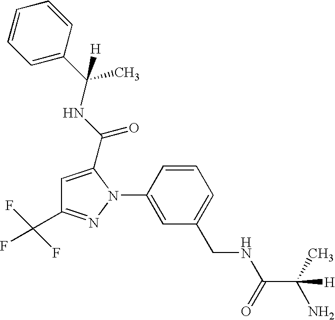 | 460.5 |
| 17 | 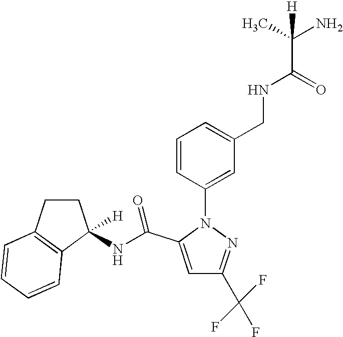 | 472.5 |
| 18 | 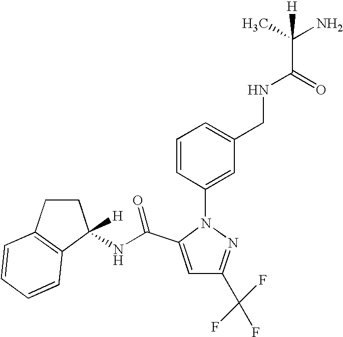 | 472.5 |
| 19 | 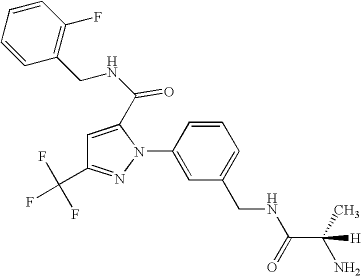 | 464.4 |
| 20 | 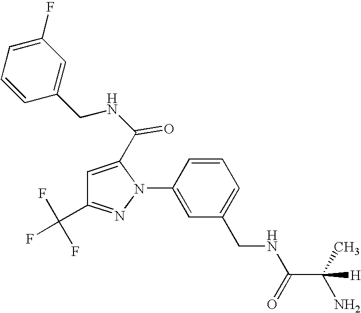 | 464.4 |
| 21 | 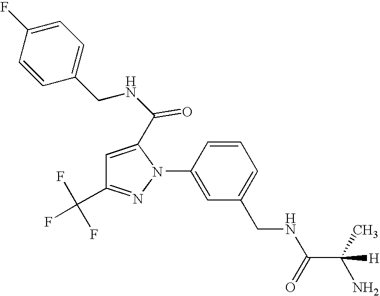 | 464.4 |
| 22 | 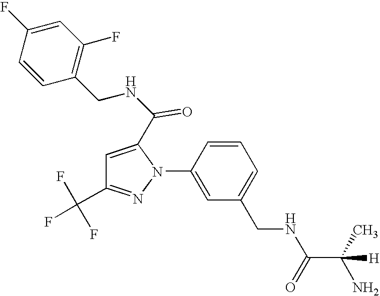 | 482.4 |
| 23 | 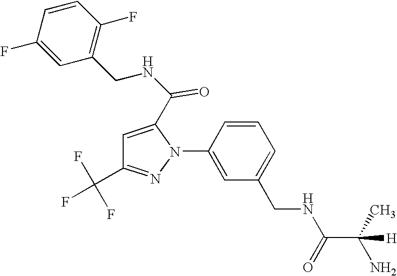 | 482.4 |
| 24 | 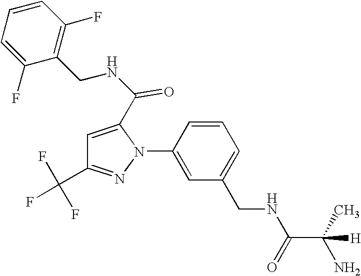 | 482.4 |
| 25 | 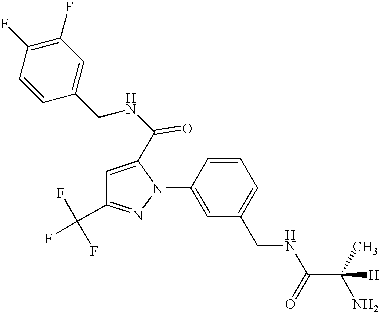 | 482.4 |
| 26 | 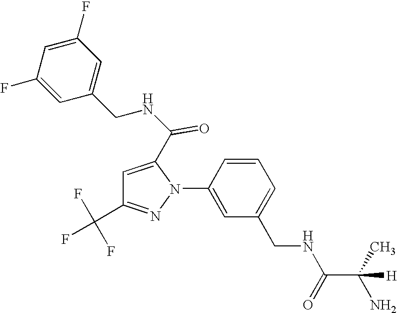 | 482.4 |
| 27 | 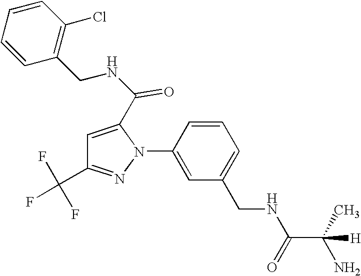 | 480.9 |
| 28 | 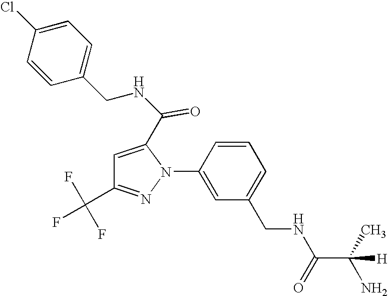 | 480.9 |
| 29 | 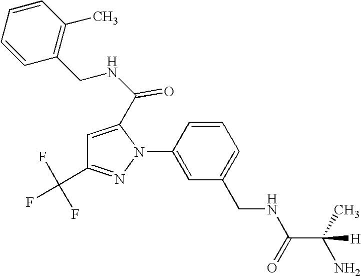 | 460.5 |
| 30 | 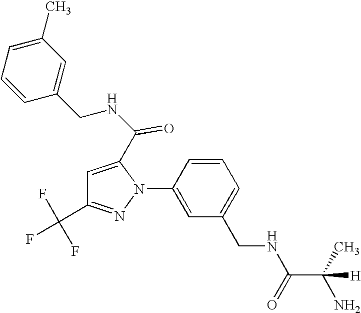 | 460.5 |
| 31 | 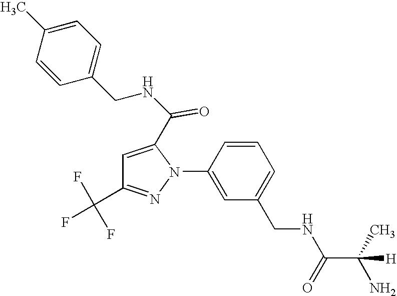 | 460.5 |
| 32 | 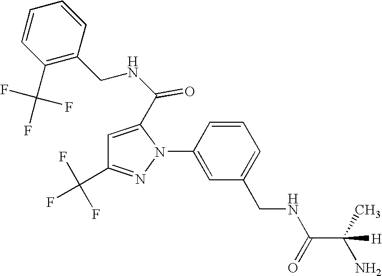 | 514.4 |
| 33 | 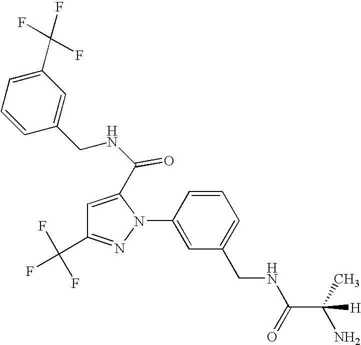 | 514.4 |
| 34 | 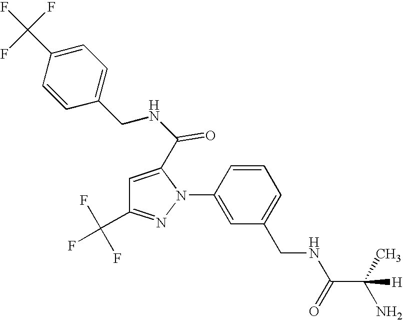 | 514.4 |
| 35 | 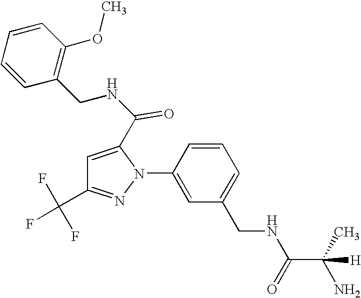 | 476.5 |
| 36 | 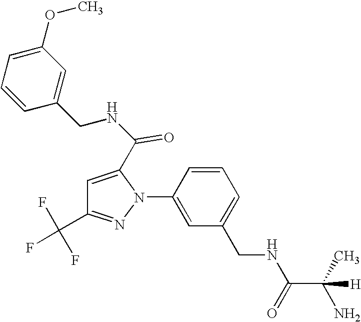 | 476.5 |
| 37 | 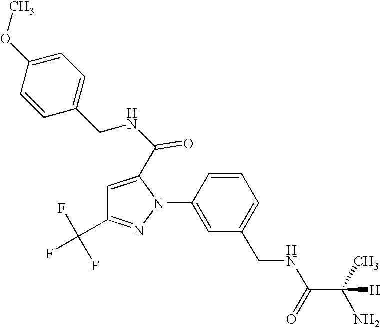 | 476.5 |
| 38 | 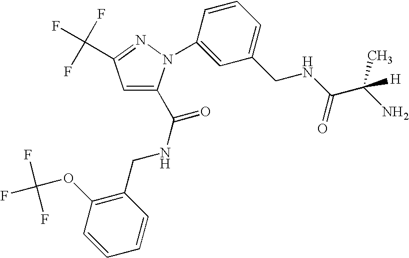 | 530.4 |
| 39 | 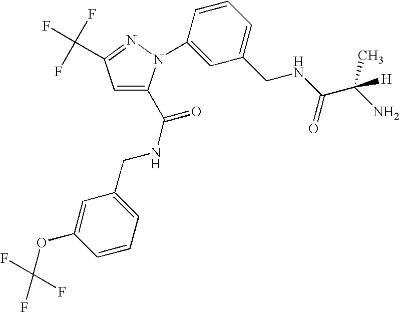 | 530.4 |
| 40 | 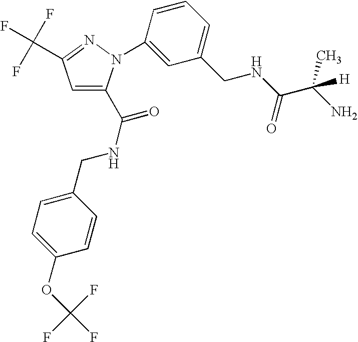 | 530.4 |
| 41 | 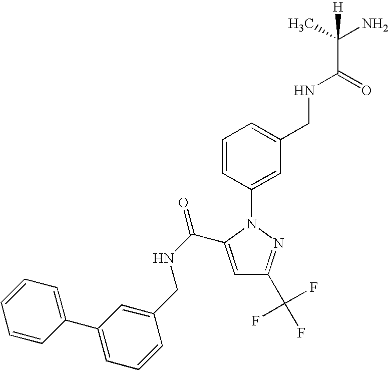 | 522.5 |
| 42 | 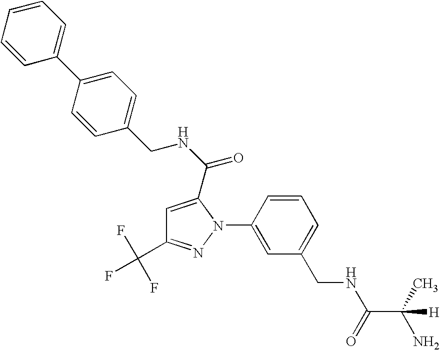 | 522.5 |
| 43 | 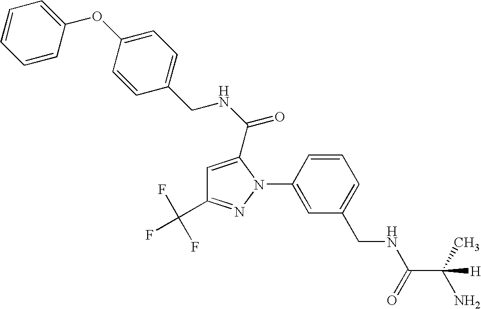 | 538.5 |
| 44 | 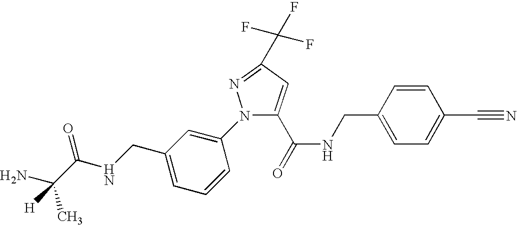 | 471.5 |
| 45 | 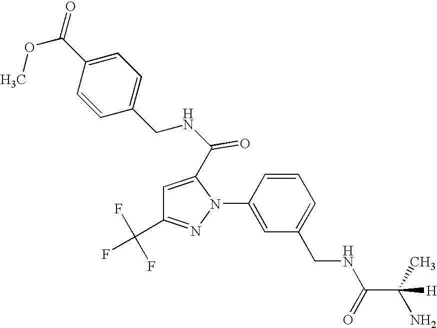 | 504.5 |
| 46 | 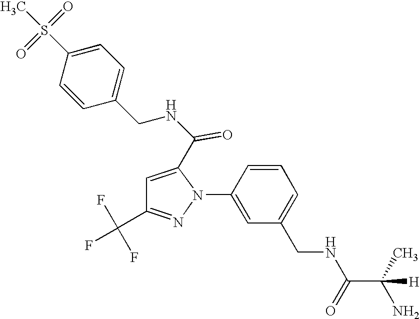 | 524.5 |
| 47 | 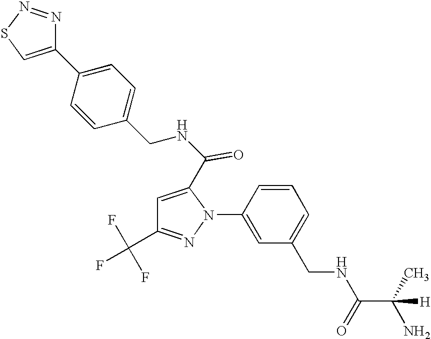 | 530.6 |
| 48 | 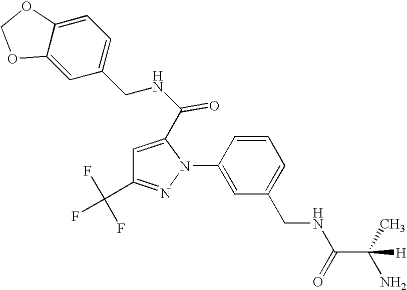 | 490.5 |
| 49 | 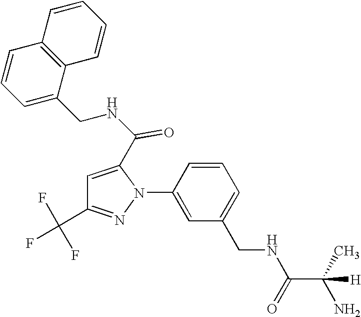 | 496.5 |
| 50 | 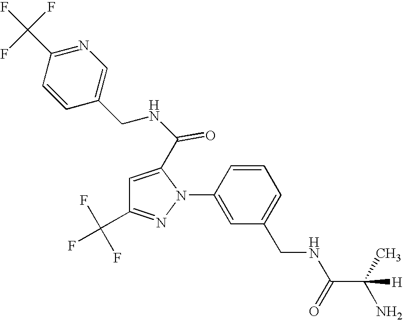 | 515.4 |
| 51 | 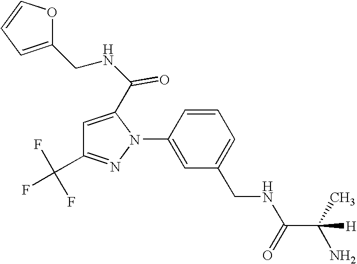 | 436.4 |
| 52 | 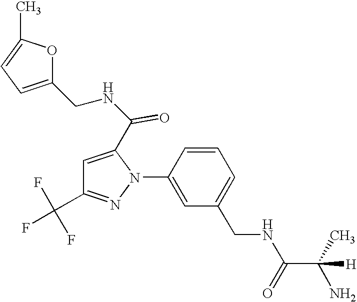 | 450.4 |
| 53 | 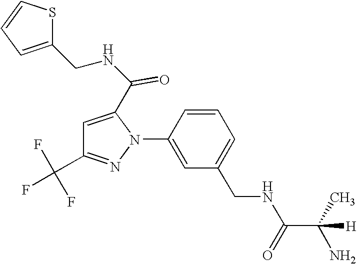 | 452.5 |
| 54 | 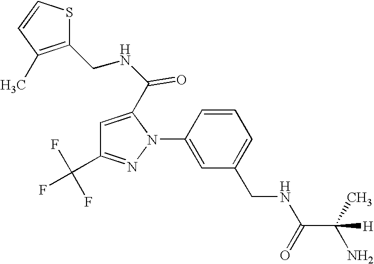 | 466.5 |
| 55 | 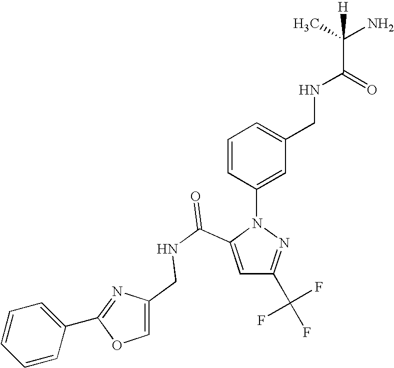 | 513.5 |
| 56 | 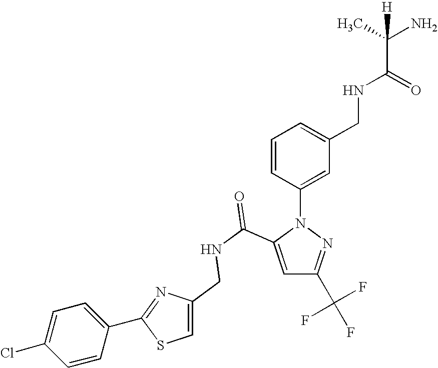 | 564 |
| 57 | 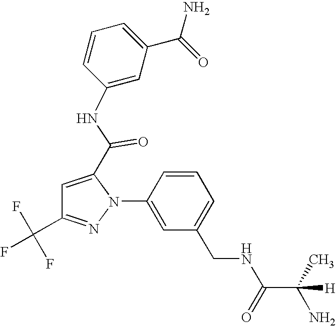 | 475.4 |
| 58 | 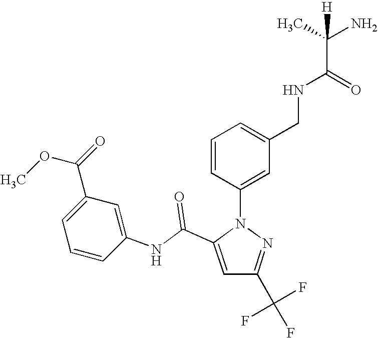 | 490.5 |
| 59 | 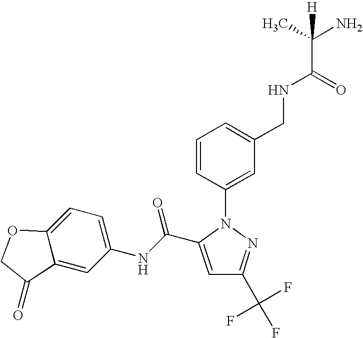 | 488.4 |
| 60 | 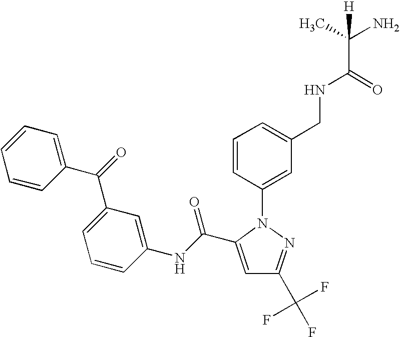 | 536.5 |
| 61 | 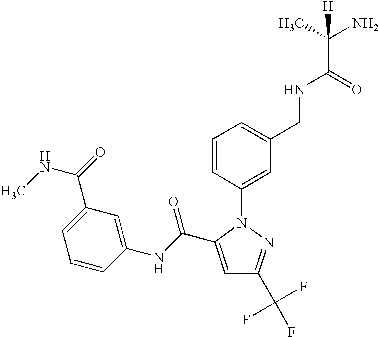 | 489.5 |
| 62 | 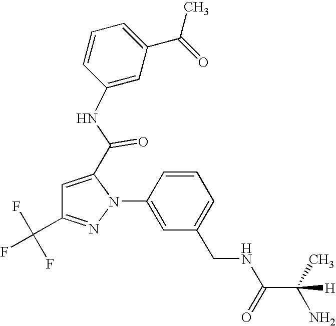 | 474.5 |
| 63 | 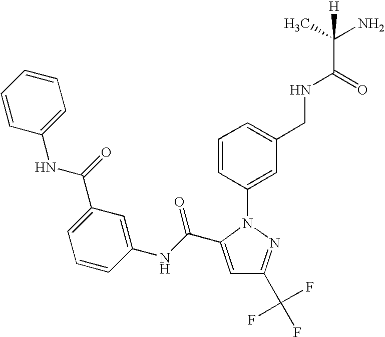 | 551.5 |
| 64 | 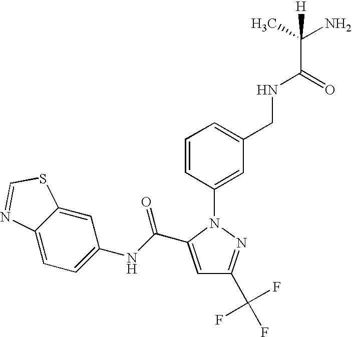 | 489.5 |
| 65 | 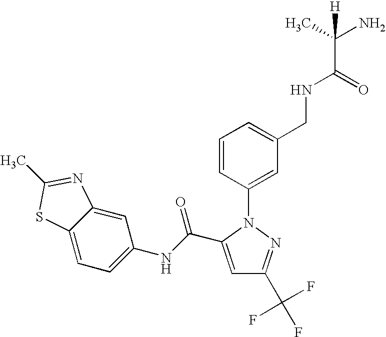 | 503.5 |
| 66 | 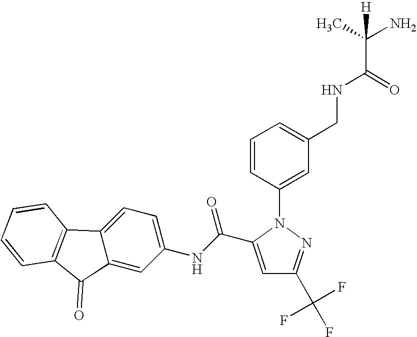 | 534.5 |
| 67 | 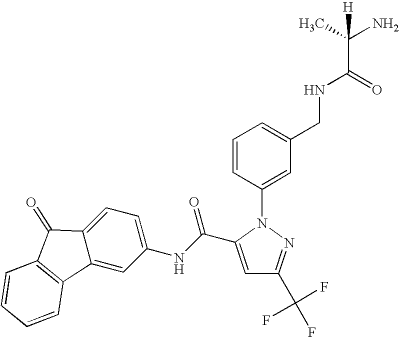 | 534.5 |
| 68 | 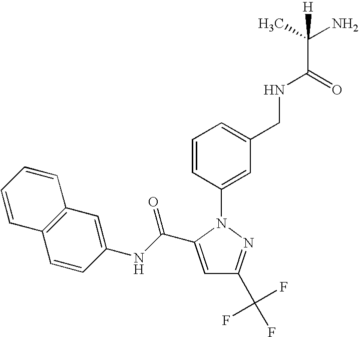 | 482.5 |
| 69 | 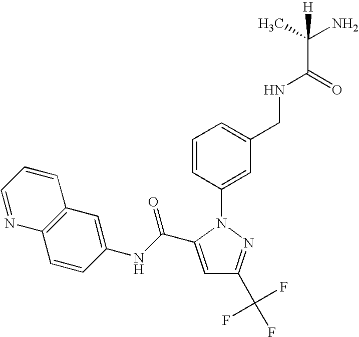 | 483.5 |
| 70 | 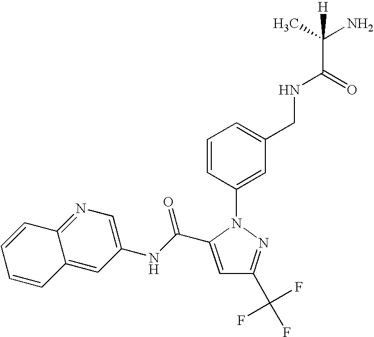 | 483.5 |
| 71 | 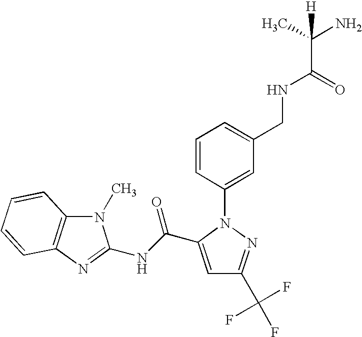 | 486.5 |
| 72 | 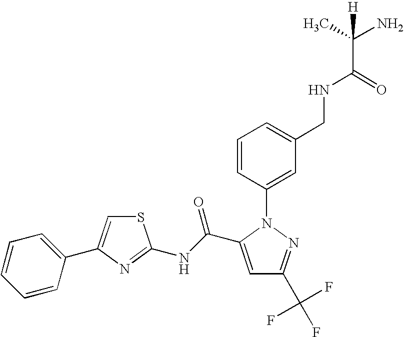 | 515.5 |
| 73 | 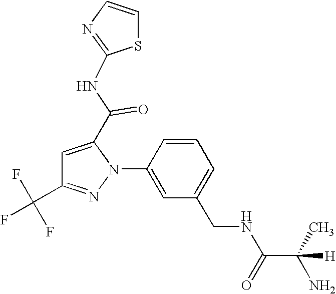 | 439.4 |
| 74 | 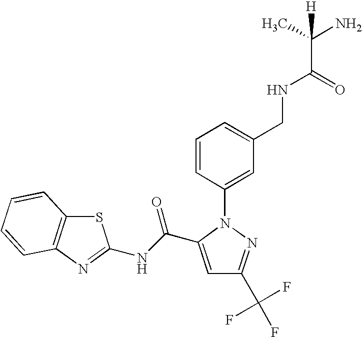 | 489.5 |
| 75 | 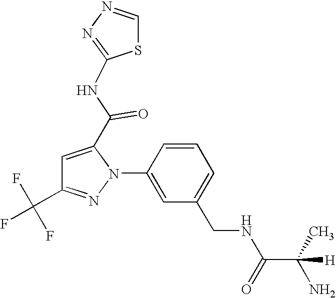 | 440.4 |
| 76 | 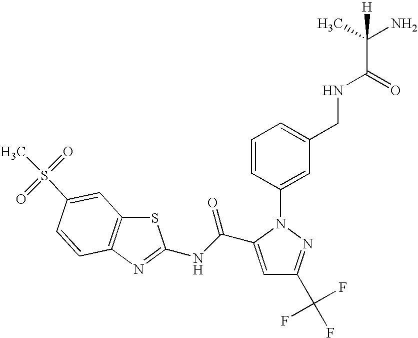 | 567.6 |
| 77 | 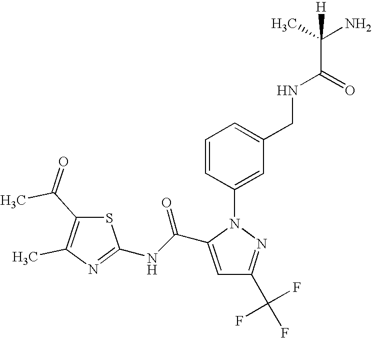 | 495.5 |
| 78 | 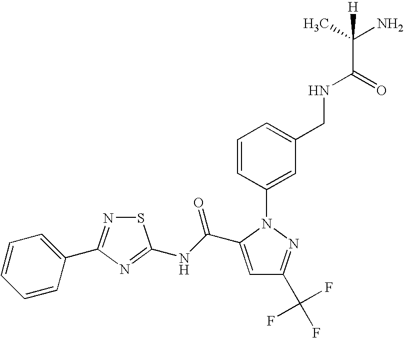 | 516.5 |
| 79 | 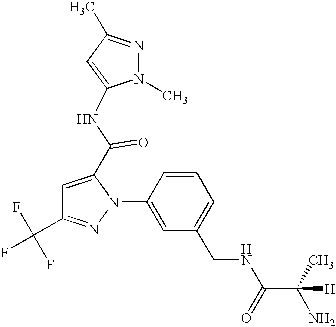 | 450.4 |
| 80 | 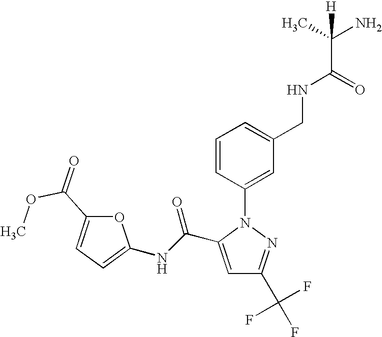 | 480.4 |
| 81 | 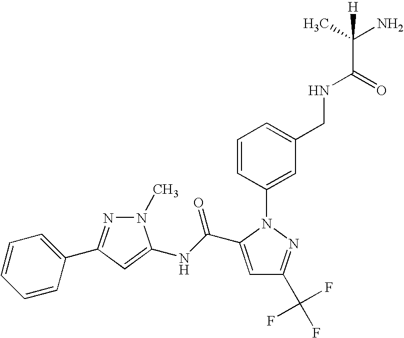 | 512.5 |
| 82 | 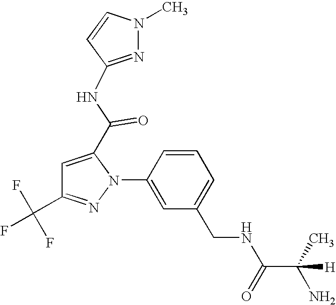 | 436.4 |
| 83 | 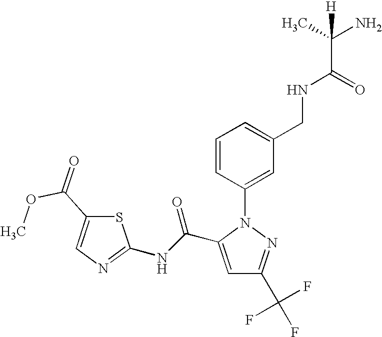 | 497.5 |
| 84 | 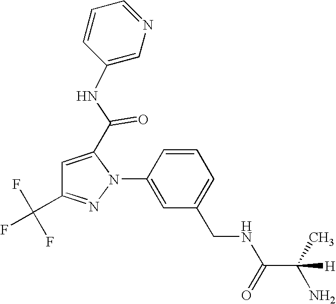 | 433.4 |
| 85 | 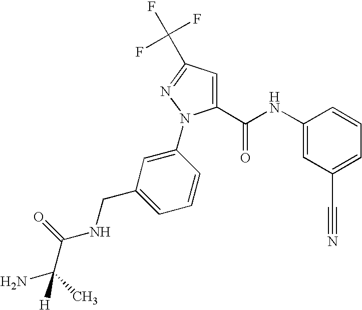 | 457.4 |

### N-(3-(5-(5-phenyl-1,3,4-oxadiazol-2-yl)-3(trifluoromethyl)-1H-pyrazol-1-yl)benzyl)-2-aminopropanamide (Example 86)

### Example 86

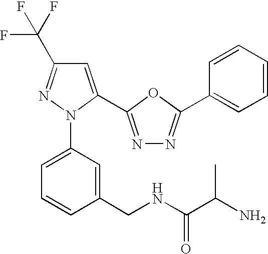

### Part A: Preparation of 1-(3-((2-(tert butoxycarbonyl)aminopropanamido)methyl)phenyl)-N′-benzoyl-3-(trifluoromethyl)-1H-pyrazole-5-carbohydrazide

PyBOP (86 mg, 0.16 mmol) was added to a solution of 1-(3-((2-(tert butoxycarbonyl)propanamido)methyl)phenyl)-3-(trifluoromethyl)-1H-pyrazole-5-carboxylic acid (50 mg, 0.11 mmol), benzoic hydrazide (23 mg, 0.16 mmol) and N,N-diisopropylethylamine (21.2 mg, 0.16 mmol) in dichloromethane (2 mL). The reaction mixture was stirred at room temperature for 18 hours. The reaction mixture was diluted with dichloromethane-water (10:2 Ml). The organic layer was washed with water, dried and concentrated to yield the intermediate which was carried as it to the next reaction. Diisopropylcarbodiimide (50 mg) was added to the solution of the intermediate (35 mg, 0.06 mmol) in DMF (0.1 Ml). The solution was heated at 100° C. for 18 hours. The solution was concentrated in vaccuo and the crude was purified using preparative HPLC using conditions below to yield 28.3 mg of tert-butyl 1-(3-(5-(5-phenyl-1,3,4-oxadiazol-2-yl)-3-(trifluoromethyl)-1H-pyrazol-1-yl)benzylamino)-1-oxopropan-2-ylcarbamate.

Conditions: Column—YMC ODS (20×50 mm)

Solvents—A—90% water—10% methanol—0.1% TFA
   B—10% water—90% methanol—0.1% TFA
Gradient—20% B to 100% B in 12 min
Retention time: 10.1 min

MS (ESI) m/z 557.21 (M+H)

NMR:8.12(m, 2H), 7.8(s, 1H), 7.6-7.5(m, 4H), 7.2-7.1 (m, 2H), 6.9(s, 1H), 4.5(dd, 2H), 4.2(, 1H), 1.46(s, 9H), 1.3(d, 3H)

### Part B: Preparation of N-(3-(5-(5-phenyl-1,3,4-oxadiazol-2-yl)-3(trifluoromethyl)-1H-pyrazol-1-yl)benzyl)-2-aminopropanamide (Example 2)

tert-butyl 1-(3-(5-(5-phenyl-1,3,4-oxadiazol-2-yl)-3-(trifluoromethyl)-1H-pyrazol-1-yl)benzylamino)-1-oxopropan-2-ylcarbamate (28.3 mg, 0.05 mmol) was treated with a mixture of trifluoroacetic acid (0.3 Ml) in CH₂Cl₂ (3 Ml). The reaction mixture was stirred at room temperature for 1 hour. The reaction mixture was concentrated in vacuo. The solution of crude product in methanol was loaded onto a pre-equilibrated SCX column (3 G in 6 ml cartridge, Waters). The column was eluted with methanol (2×5 Ml) and then with 1 M ammonia solution in methanol (2×5 mL). The fractions containing methanolic ammonia were concentrated in vacuo to give pure product (Example 2) (17 mg).

MS m/z 457.16 (M+H)

NMR (CD3OD): 7.9 (m, 2H), 7.8 (s, 1H), 7.6 (m, 3H), 7.2 (m, 2H), 7.1 (s, 1H), 4.45 (dd, 2H), 3.86 (m, 1H), 1.3 (d, 3H).

Preparation of Examples 86 to 164: The following examples were prepared using a method analogous to that used to prepare Example 87.

TABLE 2

| Compd # | Structure | MS m/z |
|---|---|---|
| 87 | 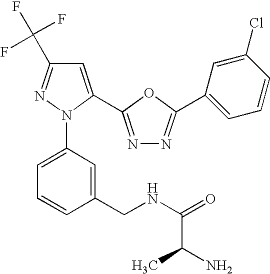 | 491.88 |
| 88 | 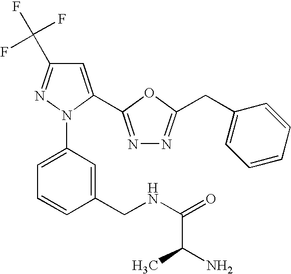 | 471.46 |
| 89 | 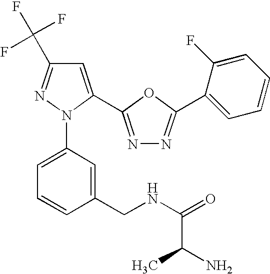 | 475.42 |
| 90 | 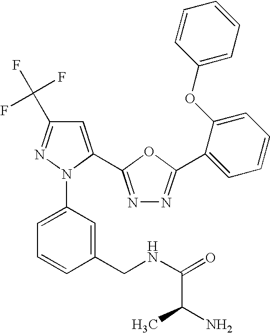 | 549.53 |
| 91 | 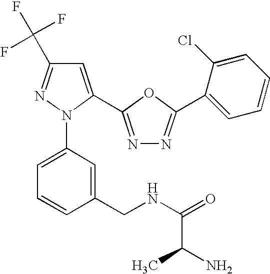 | 491.88 |
| 92 | 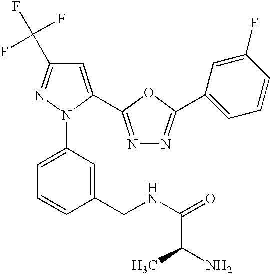 | 475.42 |
| 93 | 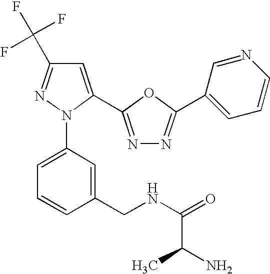 | 458.42 |
| 94 | 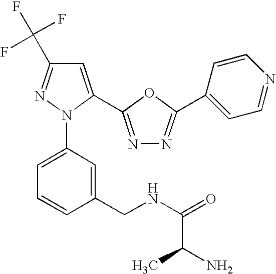 | 458.42 |
| 95 | 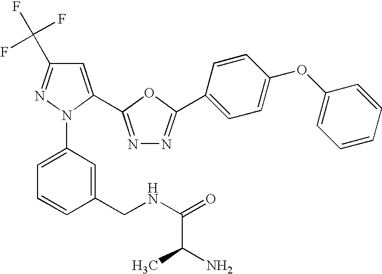 | 549.53 |
| 96 | 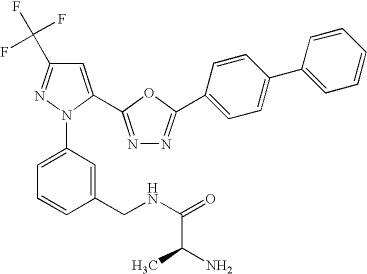 | 533.53 |
| 97 | 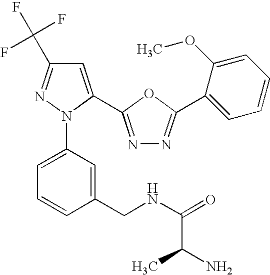 | 487.46 |
| 98 | 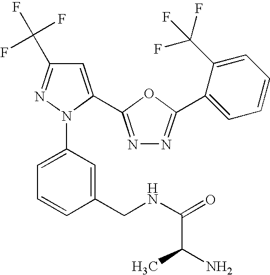 | 525.43 |
| 99 | 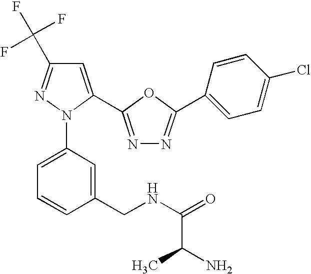 | 491.88 |
| 100 | 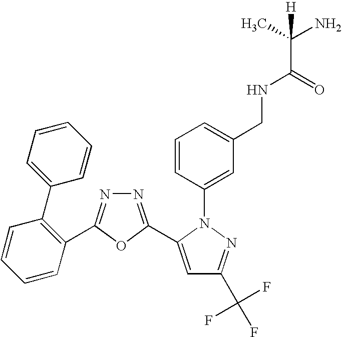 | 533.5 |
| 101 | 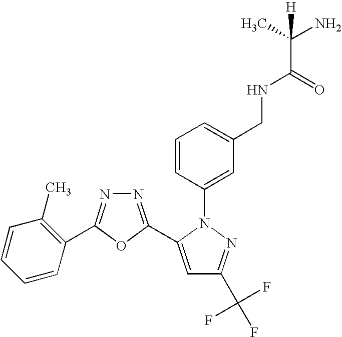 | 471.5 |
| 102 | 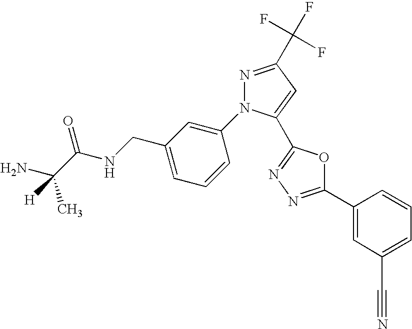 | 482.4 |
| 103 | 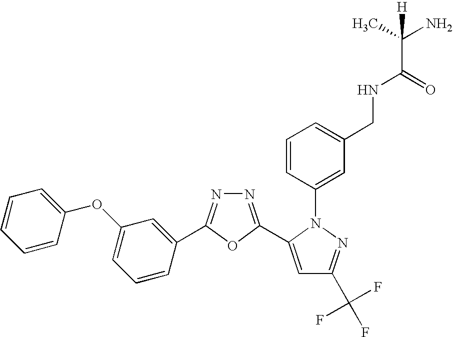 | 549.5 |
| 104 | 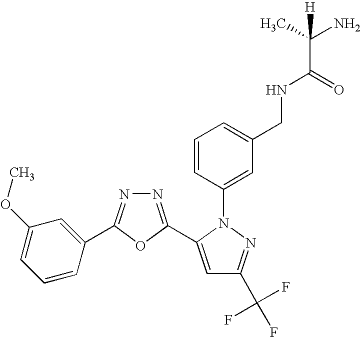 | 487.5 |
| 105 | 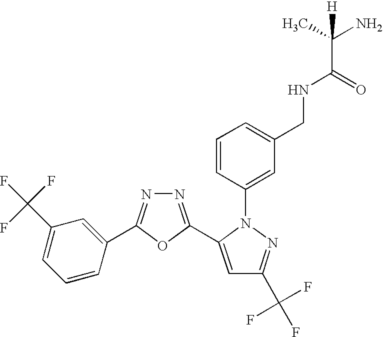 | 525.4 |
| 106 | 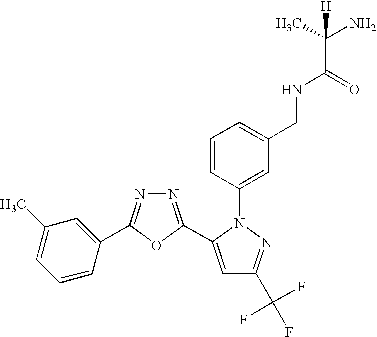 | 471.5 |
| 107 | 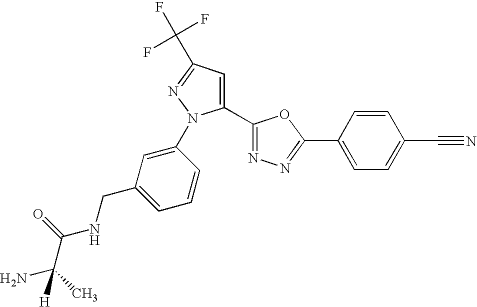 | 482.4 |
| 108 | 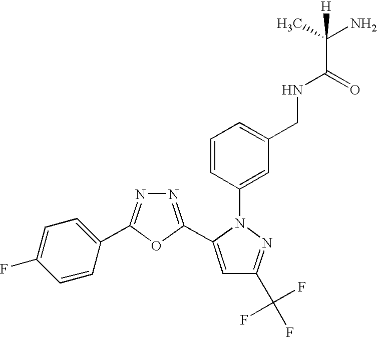 | 475.4 |
| 109 | 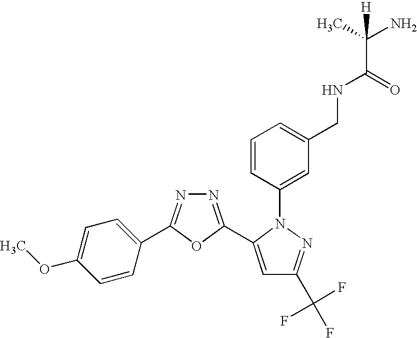 | 487.5 |
| 110 | 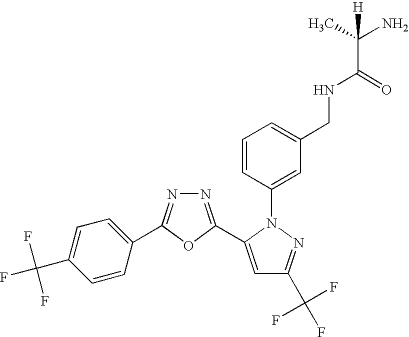 | 525.4 |
| 111 | 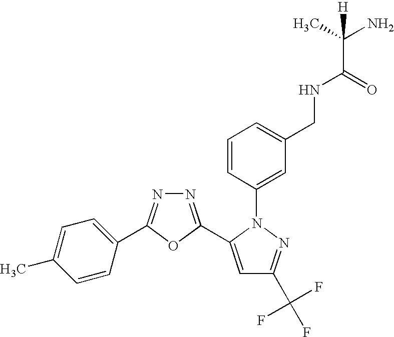 | 471.5 |
| 112 | 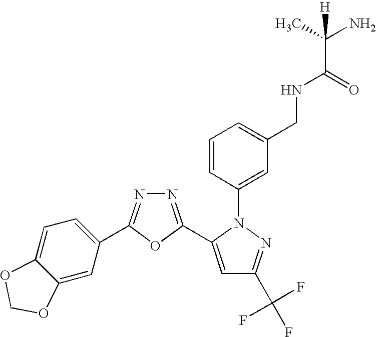 | 501.4 |
| 113 | 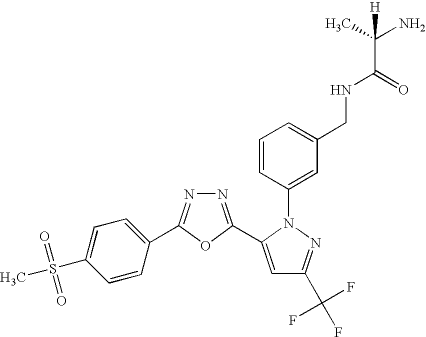 | 535.5 |
| 114 | 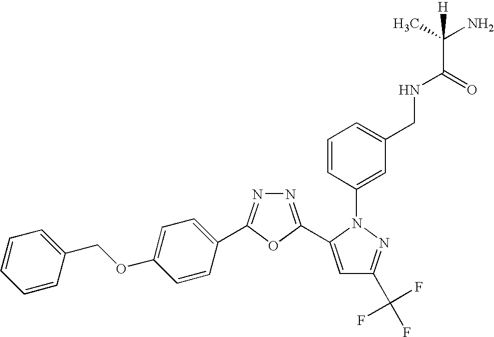 | 563.6 |
| 115 | 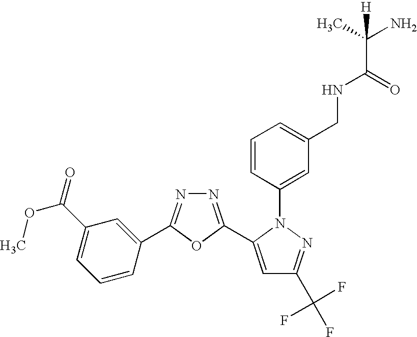 | 515.5 |
| 116 | 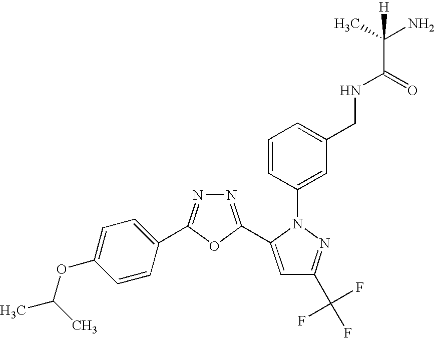 | 515.5 |
| 117 | 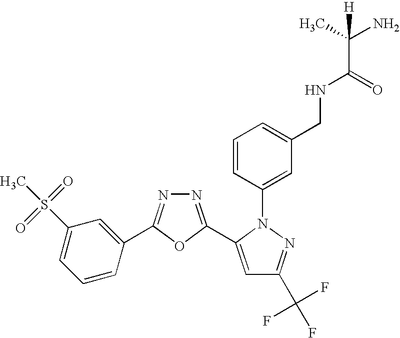 | 535.5 |
| 118 | 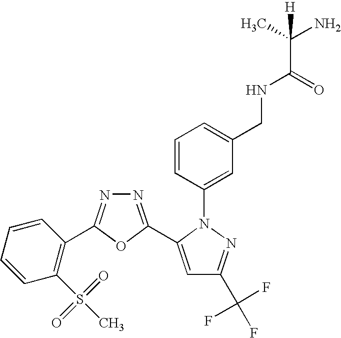 | 535.5 |
| 119 | 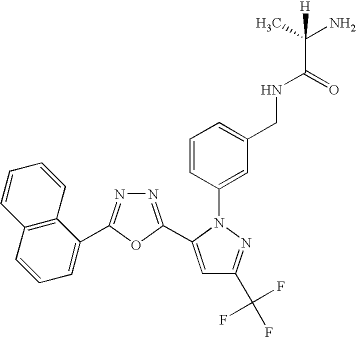 | 507.5 |
| 120 | 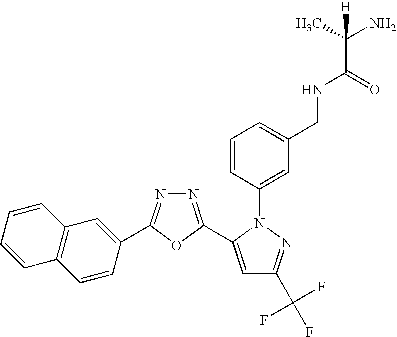 | 507.5 |
| 121 | 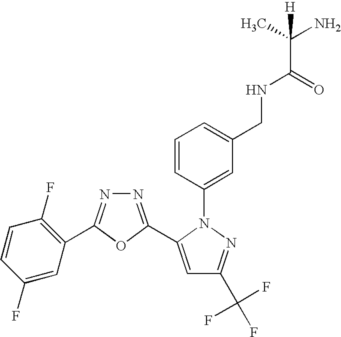 | 493.4 |
| 122 | 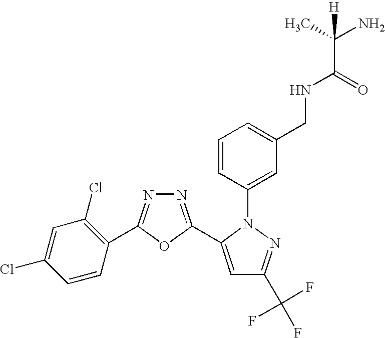 | 526.3 |
| 123 | 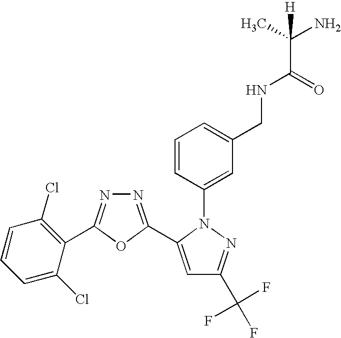 | 526.3 |
| 124 | 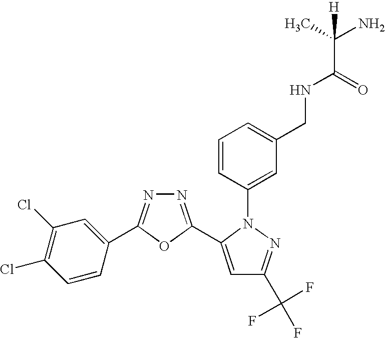 | 526.3 |
| 125 | 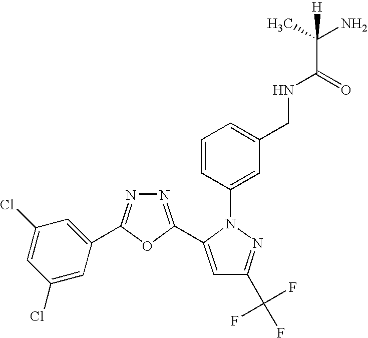 | 526.3 |
| 126 | 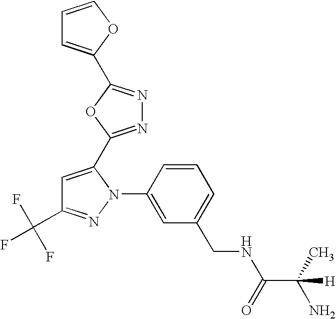 | 447.4 |
| 127 | 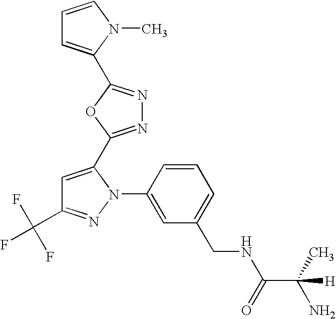 | 460.4 |
| 128 | 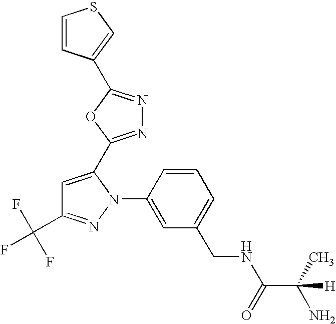 | 463.5 |
| 129 | 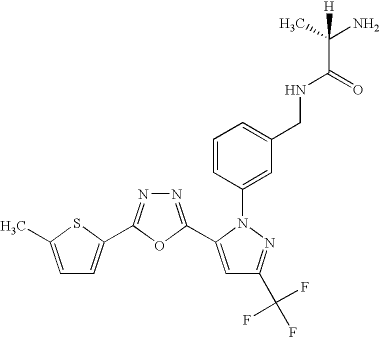 | 477.5 |
| 130 | 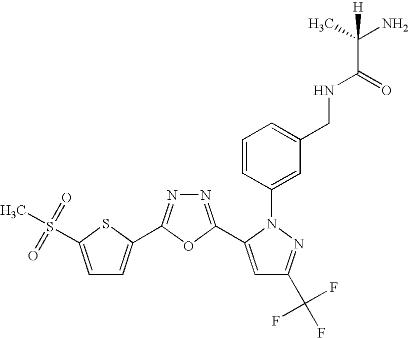 | 541.5 |
| 131 | 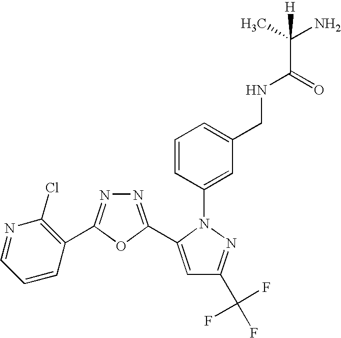 | 492.9 |
| 132 | 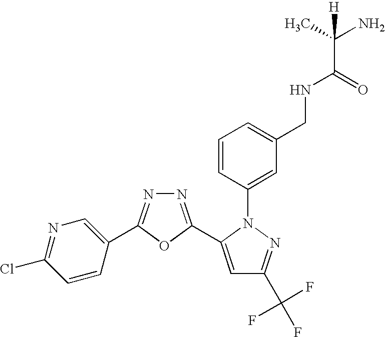 | 492.9 |
| 133 | 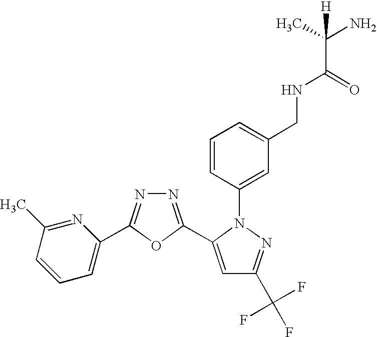 | 472.4 |
| 134 | 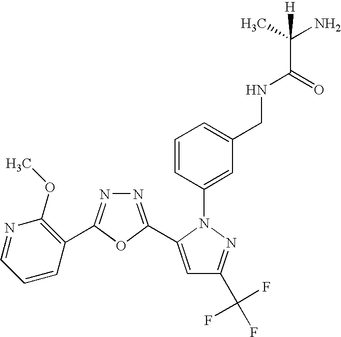 | 488.4 |
| 135 | 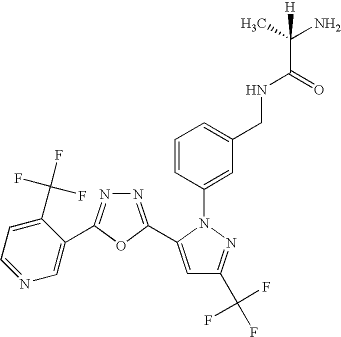 | 526.4 |
| 136 | 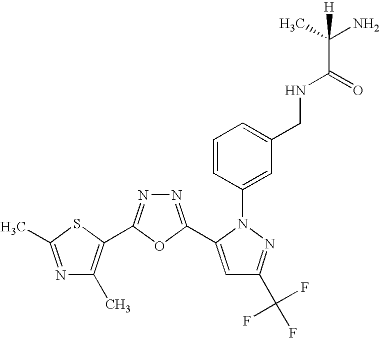 | 492.5 |
| 137 | 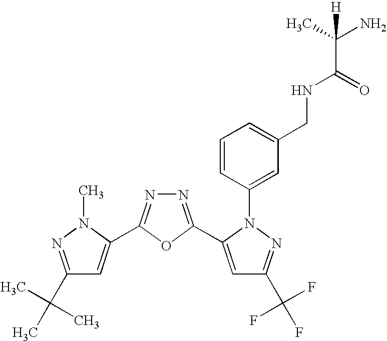 | 517.5 |
| 138 | 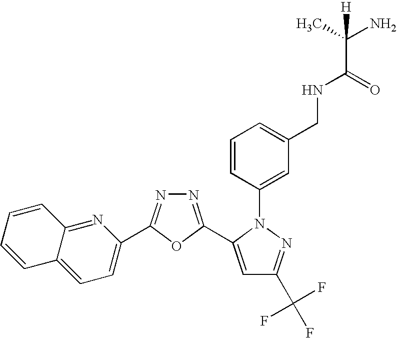 | 508.5 |
| 139 | 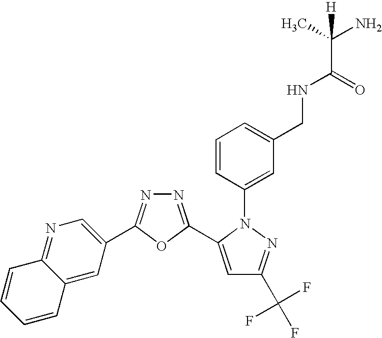 | 508.5 |
| 140 | 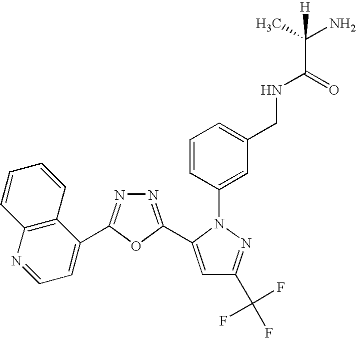 | 508.5 |
| 141 | 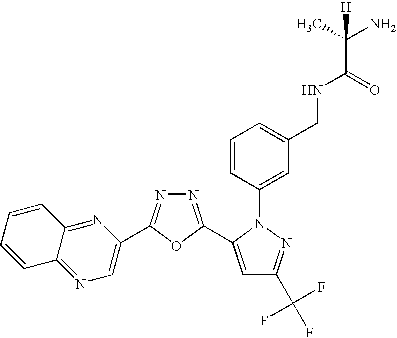 | 509.5 |
| 142 | 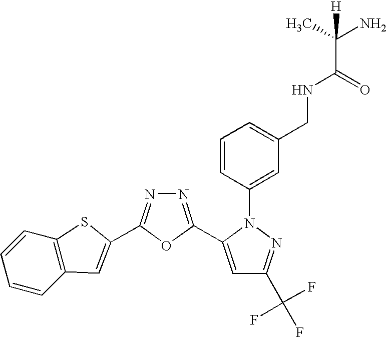 | 513.5 |
| 143 | 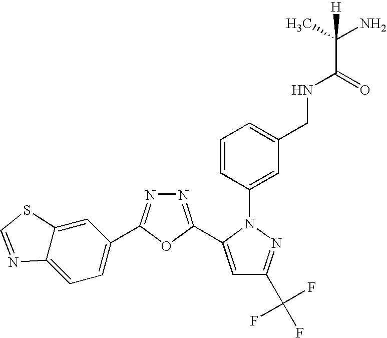 | 514.5 |
| 144 | 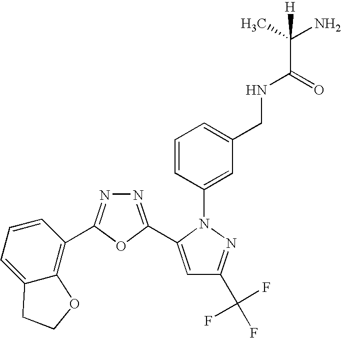 | 499.5 |
| 145 | 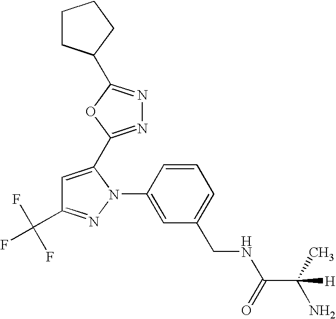 | 449.5 |
| 146 | 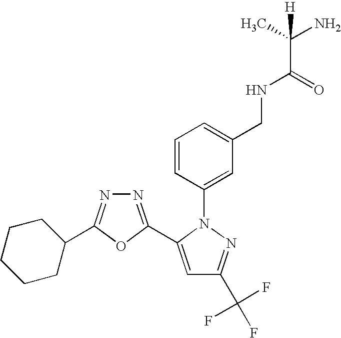 | 463.5 |
| 147 | 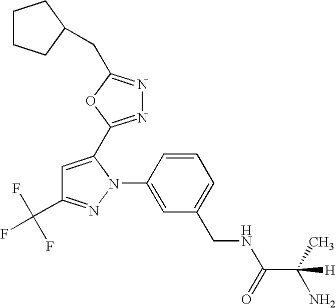 | 463.5 |
| 148 | 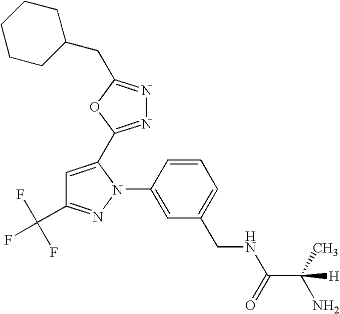 | 477.5 |
| 149 | 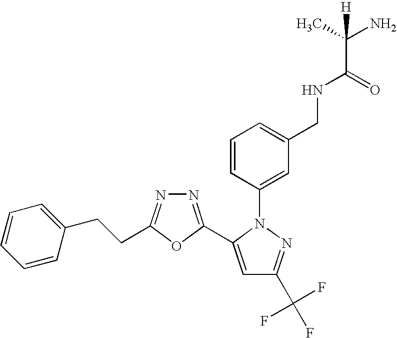 | 485.5 |
| 150 | 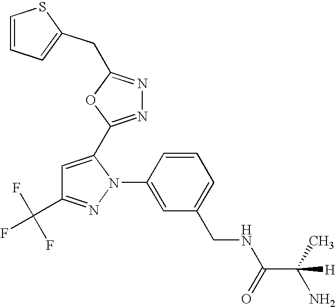 | 477.5 |
| 151 | 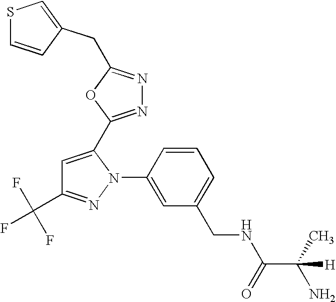 | 477.5 |
| 152 | 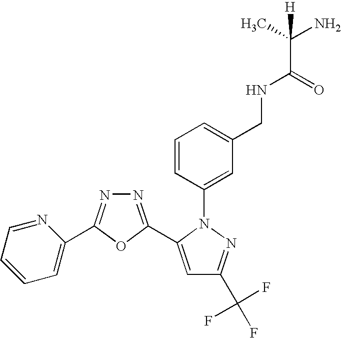 | 458.4 |
| 153 | 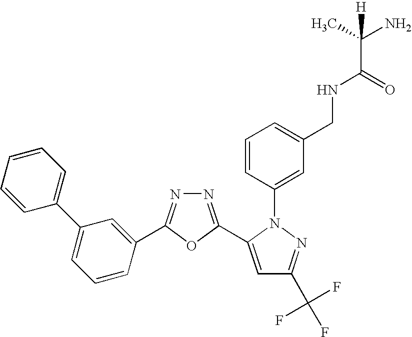 | 533.5 |
| 154 | 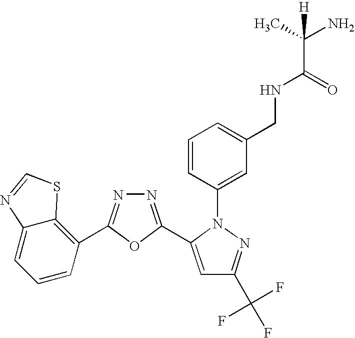 | 514.5 |
| 155 | 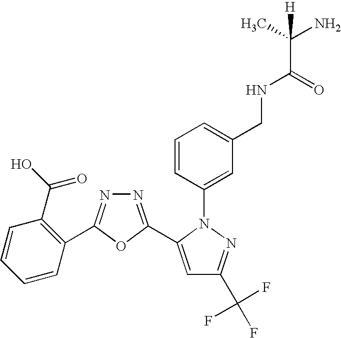 | 501.44 |
| 156 | 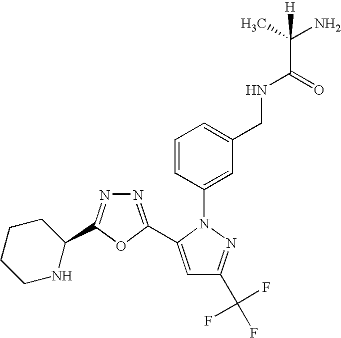 | 464.47 |
| 157 | 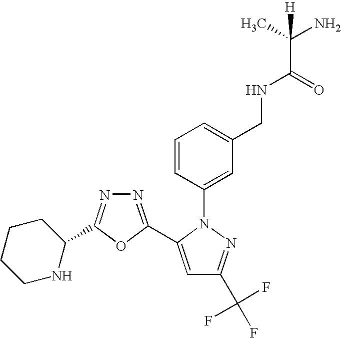 | 464.47 |
| 158 | 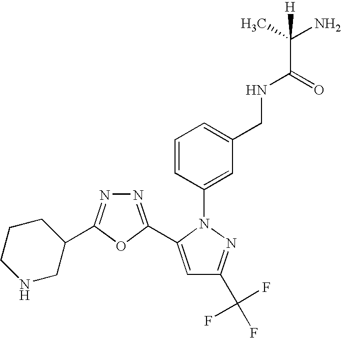 | 464.47 |
| 159 | 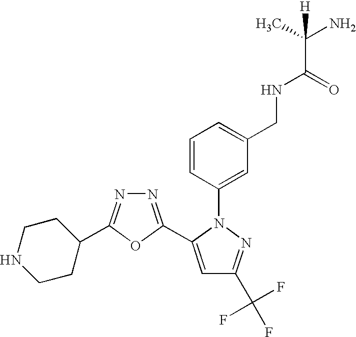 | 464.47 |
| 160 | 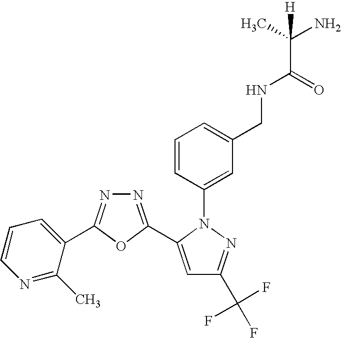 | 472.45 |
| 161 | 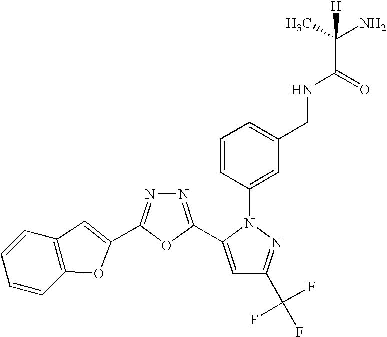 | 497.45 |
| 162 | 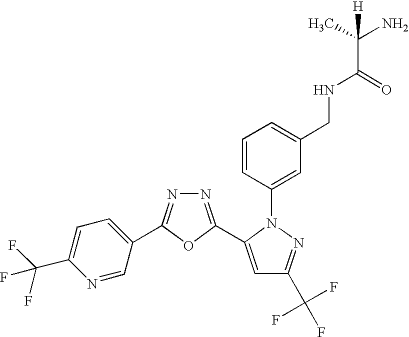 | 526.42 |
| 163 | 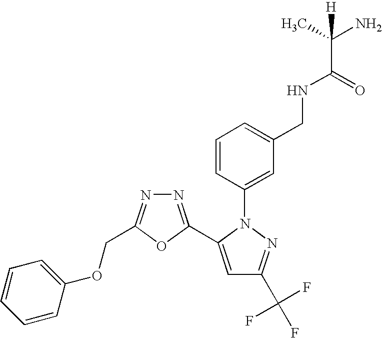 | 487.46 |
| 164 | 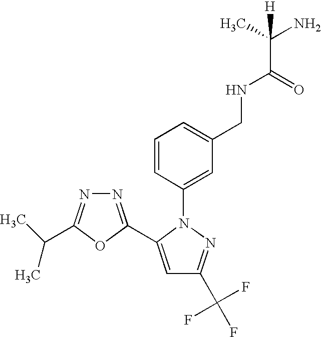 | 423.41 |
| 165 | 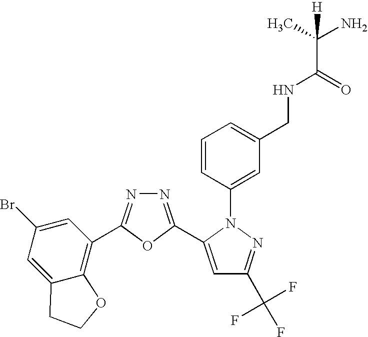 | 578.36 |

### N-(3-(5-(3-phenyl-1,2,4-oxadiazol-5-yl)-3-(trifluoromethyl)-1H-pyrazol-1-yl)benzyl)-2-aminopropanamide (Example 166)

### Example 166

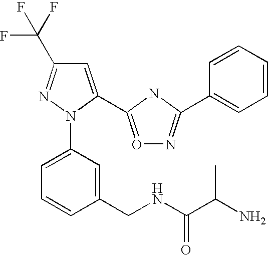

### Part A: Preparation of tert-butyl 1-(3-(5-(3-phenyl-1,2,4-oxadiazol-5-yl)-3-(trifluoromethyl)-1H-pyrazol-1-yl)benzylamino)-1-oxopropan-2-ylcarbamate

Benzamidoime (16.4 mg, 0.12 mmol) was added to a solution of 1,1′-carbonyldiimidazole (20 mg, 0.12 mmol) and 1-(3-((2-(tert butoxycarbonyl)propanamido)methyl)phenyl)-3-(trifluoromethyl)-1H-pyrazole-5-carboxylic acid (50 mg, 0.1 mmol) in DMF (1 Ml). The reaction mixture was stirred at room temperature for 4 hours. Additional 1,1′-carbonyldiimidazole (19.5 mg, 0.12 mmol) in DMF (1 Ml) was added to the reaction mixture. The mixture was heated to 100° C. for 6 hours. The solution was concentrated in vacuo and the crude product was purified using preparative HPLC to yield tert-butyl 1-(3-(5-(3-phenyl-1,2,4-oxadiazol-5-yl)-3-(trifluoromethyl)-1H-pyrazol-1-yl)benzylamino)-1-oxopropan-2-ylcarbamate (2.3 mg).

Conditions: Column—YMC ODS (20×50 mm)

Solvents—A—90% water—10% methanol—0.1% TFA
   B—10% water—90% methanol—0.1% TFA
Gradient—20% B to 100% B in 12 min
Retention time: 10.2 min

MS (ESI) m/z 557.21 (M+H)

NMR: 8.1(m, 2H), 7.8(s, 1H), 7.5-7.6(m,4H), 7.1(m, 1H), 7.24(m,1H), 7.0(s,1H), 4.55(m,2H), 4.2(m,1H), 1.48(s,9H), 1.3(d,3H)

### Part B: Preparation of N-(3-(5-(3-phenyl-1,2,4-oxadiazol-5-yl)-3-(trifluoromethyl)-1H-pyrazol-1-yl)benzyl)-2-aminopropanamide (Example 3)

tert-butyl 1-(3-(5-(3-phenyl-1,2,4-oxadiazol-5-yl)-3-(trifluoromethyl)-1H-pyrazol-1-yl)benzylamino)-1-oxopropan-2-ylcarbamate (2.3 mg, 0.004 mmol) was treated with trifluoroacetic acid (0.1 Ml) in CH₂Cl₂ (1 mL). The reaction mixture was stirred at room temperature for 1 hour. The reaction mixture was concentrated in vacuo. The solution of crude product in methanol was loaded onto a pre-equilibrated SCX column (3 G in 6 ml cartridge, Waters). The column was eluted with methanol (2×5 Ml) and then with 1 M ammonia solution in methanol (2×5 mL). The fractions containing methanolic ammonia were concentrated in vacuo to give title product (Example 3) (1 mg).

MS (ESI) 457.16 (M+H)

NMR(CD3OD): 8.1 (d, 2H), 7.8 (s, 1H), 7.6 (m, 3H), 7.0-7.2 (m, 3H), 4.4 (s, 2H), 3.5 (s, 1H), 1.3 (d, 2H).

### N-(3-(5-(5-(phenylamino)-1,3,4-oxadiazol-2-yl)-3-(trifluoromethyl)-1H-pyrazol-1-yl)benzyl)-2aminopropanamide (Example 167)

### Example 167

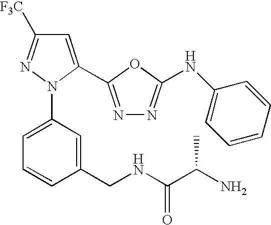

### Part A: Preparation of ethyl 1-(3-cyanophenyl)-3-(trifluoromethyl)-1H-pyrazole-5-carboxylate

A flask was charged with 1-(3-(aminomethyl)phenyl)-3-(trifluoromethyl)-1H-pyrazole-5-carboxylic acid (5.5 g) in dichloromethane (25 mL), and oxalyl chloride (12 mL) was added to the flask (and two drops of DMF). The solution was stirred at RT for 1 hour, concentrated in vacuo and re-dissolved in dichlormethane (10 mL) and ethanol (10 mL). The solution was stirred at RT for 2 hours. The solution was concentrated in vacuo and dried by vacuum pump to yield 4.5 G of the title ester.

MS m/z 310.2 (M+H)

### Part B: Preparation of ethyl 1-(3-(aminomethyl)phenyl)-3-(trifluoromethyl)-1H-pyrazole-5-carboxylate

A flask was charged with ethyl 1-(3-cyanophenyl)-3-(trifluoromethyl)-1H-pyrazole-5-carboxylate (2.8 g) in ethanol (40 mL) at RT. Palladium on carbon (1.0 g) and platinum oxide (350 mg), and HCl in water (0.5 mL) was added to the flask. The reaction solution was stirred at RT with hydrogen gas from a balloon for 2 hours. The solution was filtered through celite, washed with methanol (10 mL) and concentrated. The crude was purified by silica gel column chromatography with 5% methanol and CH₂Cl₂ giving the pure 1.2 G of product.

MS m/z 314.19 (M+H)

### Part C: Preparation of ethyl 1-(3-(((S)-2-(tert-butoxycarbonyl)propanamido)methyl)phenyl)-3-(trifluoromethyl)-1H-pyrazole-5-carboxylate

A flask was charged with ethyl 1-(3-(aminomethyl)phenyl)-3-(trifluoromethyl)-1H-pyrazole-5-carboxylate (1.20 g) in CH₂Cl₂ (10 mL) at RT. N-Boc-alanine succinamide (1.1 g) was added to the reaction solution along with triethylamine (0.53 mL). The reaction solution was stirred at RT for 2 hours. The solution was concentrated in vaccuo The crude was purified using silica gel column chromatography (5% ethyl acetate/heptane) to give 1.7 G of the pure product.

MS m/z 485 (M+H)

### Part D: Preparation of 1-(3-(((S)-2-(tert-butoxycarbonyl)propanamido)methyl)phenyl)-3-(trifluoromethyl)-1H-pyrazole-5-carboxyhydrazide

A flask was charged with ethyl 1-(3-(((S)-2-(tert-butoxycarbonyl)propanamido)methyl)phenyl)-3-(trifluoromethyl)-1H-pyrazole-5-carboxylate (1.78 G), hydrazine monohydrate (0.445 mL) in ethanol (10 mL). The solution was heated to 60° C. for 6 hours The solution was concentrated in vacuo to a white powder, aqueous work up with CH₂Cl₂ and H2O, dried with magnesium sulfate and concentrated giving 1.7 G of the pure product.

MS m/z 471 (M+H)

### Part E: tert-butyl-1-(3-(5-(5-(phenylamino)-1,3,4-oxadiazol-2-yl)-3-(trifluoromethyl)-1H-pyrazol-1-yl)benzylamino)-1-oxopropan-2-ylcarbamate

A vial was charged with 1-(3-(((S)-2-(tert-butoxycarbonyl)propanamido)methyl)phenyl)-3-(trifluoromethyl)-1H-pyrazole-5-carboxyhydrazide (50 mg), phenylisothiocyanate (16 mg, 14 μL)) and DMF (3 mL). The reaction solution was stirred at RT for 2 hours. To the solution was added 3 ml of DMF and PS-Carbodiimide (PS=polystyrene) (483 mg) and the solution was heated to 80° C. for 24 hours. To the solution was added PS-BEMP and P-propylamine and the solution was shaken at RT for 24 hours. The solution was cooled, filtered and purified using preparative HPLC using conditions given below
Conditions: Column—YMC ODS (20×50 mm)
Solvents—A—90% water—10% methanol—0.1% TFA
   B—10% water—90% methanol—0.1% TFA
Gradient—45% B to 100% B in 12 min
Retention time: 10.2 min

MS m/z 572 (M+H)

NMR (CD3OD): 7.5-7.1 (m,9H), 6.95 (t, 1H), 4.39 (dd, 2H), 3.94 (m, 1H), 1.45-1.13 (m, 12H)

### Part F: Preparation of N-(3-(5-(5-(phenylamino)-1,3,4-oxadiazol-2-yl)-3-(trifluoromethyl)-1H-pyrazol-1-yl)benzyl)-2-aminopropanamide (Example 4)

A flask was charged with tert-butyl-1-(3-(5-(5-(phenylamino)-1,3,4-oxadiazol-2-yl)-3-(trifluoromethyl)-1H-pyrazol-1-yl)benzylamino)-1-oxopropan-2-ylcarbamate (31 mg) in dichloromethane (3 mL) and TFA (0.3 mL). The reaction solution was stirred at RT for 1 hour. The solvent and excess TFA was removed in vacuo. The product was converted into the free base by passing through an SCX column (500 mg) eluting with 2 M ammonia solution in methanol to give 21 mg of the title product.

MS m/z 472 (M+H)

NMR (CD3OD):7.2-7.5(m,9H), 6.96(t,1H), 4.42(dd,2H), 3.88(m,1H), 1.41(d,3H)

Preparation of Examples 168 to 177: The following examples were prepared using a method analogous to that used to prepare Example 166.

TABLE 3

| Compd # | Structure | MS m/z |
|---|---|---|
| 168 | 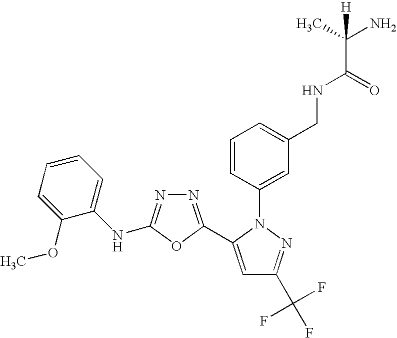 | 502.47 |
| 169 | 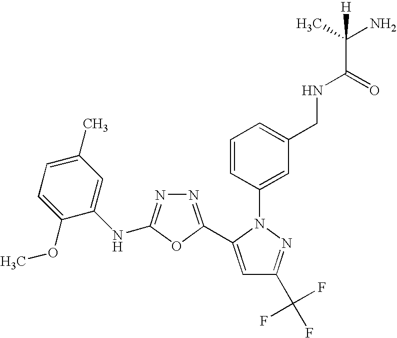 | 516.5 |
| 170 | 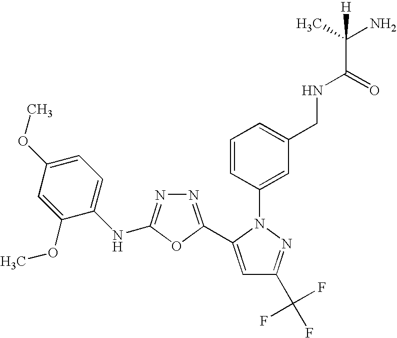 | 532.5 |
| 171 | 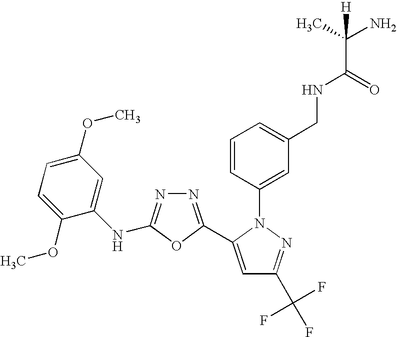 | 532.5 |
| 172 | 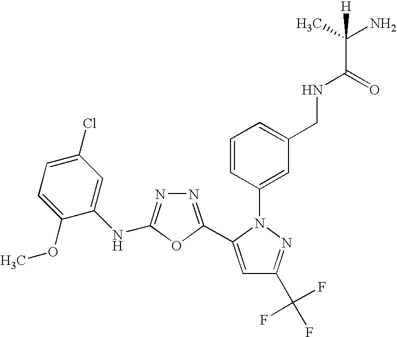 | 536.92 |
| 173 | 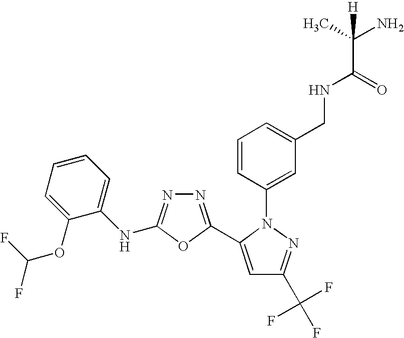 | 538.45 |
| 174 | 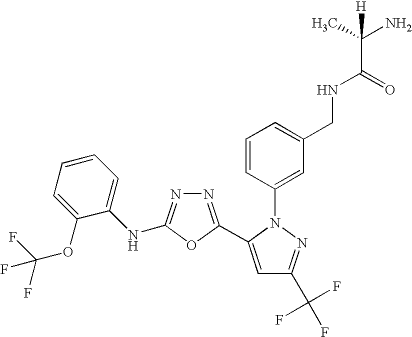 | 556.44 |
| 175 | 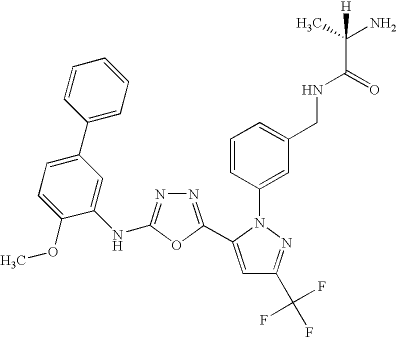 | 578.57 |
| 176 | 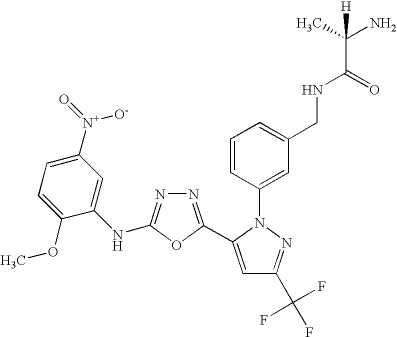 | 547.47 |
| 177 | 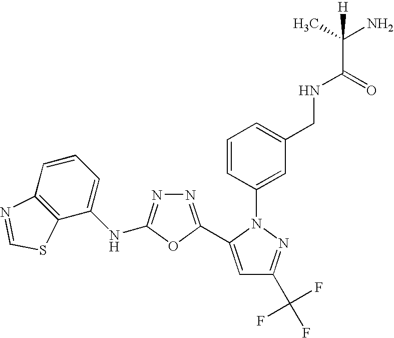 | 529.52 |

### N-(3-(5-(benzo[d]oxazol-2-yl)-3-(trifluoromethyl)-1H-pyrazol-1-yl)benzyl)-2-aminopropanamide (Example 178)

### Example 178

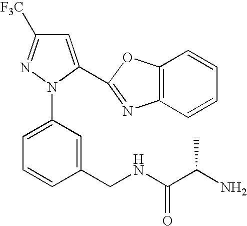

### Part A: Preparation of tert-butyl-1-(3-(5-((2-hydroxyphenyl)carbamoyl)-3-(trifluoromethyl)-1H-pyrazol-1-yl)benzylamino)-1-oxopropan-2-ylcarbamate

A 10 mL vial was charged with 1-(3-((2-(tert butoxycarbonyl)propanamido)methyl)phenyl)-3-(trifluoromethyl)-1H-pyrazole-5-carboxylic acid (170 mg, 0.37 mmol), PyBrOP (286 mg, 0.55, Hunig base (0.16 Ml, 0.93 mmol) in 1,4-dioxane (5 mL) followed by 2-aminophenol (60 mg, 0.55 mmol). The solution was heated to 80° C. for 1 hour and then cooled to room temperature and concentrated in vaccuo. The crude product was purified using silica gel chromatography (25% ethyl acetate-hexane) to yield 150 mg of the title compound.

MS (ESI) m/z 548 (M+H)

### Part B: Preparation of N-(3-(5-(benzo[d]oxazol-2-yl)-3-(trifluoromethyl)-1H-pyrazol-1-yl)benzyl)-2-aminopropanamide (Example 5)

DEAD (0.6 Ml, 0.32 mmol) was added to the stirred suspension of tert-butyl-1-(3-(5-((2-hydroxyphenyl)carbamoyl)-3-(trifluoromethyl)-1H-pyrazol-1-yl)benzylamino)-1-oxopropan-2-ylcarbamate (150 mg, 0.27 mmol), PS-triphenylphosphine (190 mg of 3 mmol/g loading) in THF (3 Ml. The reaction mixture was stirred at 85° C. for 10 hours. The solution was filtered and the filtrate was concentrated in vaccuo. The crude product was purified using preparative HPLC using condition shown below
Conditions: Column—YMC ODS (20×50 mm)
Solvents—A—90% water—10% methanol—0.1% TFA
   B—10% water—90% methanol—0.1% TFA
Gradient—40% B to 100% B in 10 min
Retention time: 10.2 min

MS m/z 530.1 (M+H)

The product obtained was dissolved in a mixture of CH₂Cl₂-TFA (1:0.2 Ml). The reaction mixture was stirred at room temperature for 1 hour and then concentrated in vaccuo. The product was converted to free amine by loading the methanolic solution on a SCX cartridge (500 mg) and eluting with 2M ammonia solution in methanol. The combined fractions were concentrated in vaccuo to yield 8 mg of the title product.

MS m/z 430 (M+H)

NMR (CD3OD): 7.32-7.7 (m, 9H), 4.5 (s, 2H), 3.7 (m,1H), 1.4 (d,3H).

### (2S)—N-(3-(2-methyl-5-(5-phenyl-1,3,4-oxadiazol-2yl)pyrimidin-4-yl)benzyl)-2-aminopropanamide (Example 179)

### Example 179

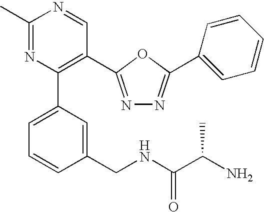

### Part A: Preparation of ethyl 2-(3-bromobenzoyl)-3-(dimethylamino)acrylate

A mixture containing ethyl 3-(3-bromophenyl)-3-oxopropanoate (4 G) and N,N-dimethylformamide dimethyl acetal (16 mL) was heated to 90° C. for 6 h. Reaction mixture was concentrated in vaccuo to give 6 G of ethyl 2-(3-bromobenzoyl)-3-(dimethylamino)acrylate crude which was used as such in the next reaction.

### Part B: Preparation of ethyl 4-(3-bromophenyl)-2-methylpyrimidine-5-carboxylate

A solution of sodium ethoxide in ethanol (1.6 Ml, 25%, 6.4 mmol) was added to the suspension of acetaamidine hydrochloride (0.6 G, 6.4 mmol) in ethanol. The mixture was stirred for 30 minutes. To this mixture was added the solution of ethyl 2-(3-bromobenzoyl)-3-(dimethyl-amino)acrylate (2 G, 1.5 mmol) in ethanol (8 Ml). The reaction mixture was heated to reflux for 3 h and then cooled to room temperature and concentrated under vaccuo. The residue was taken up in a mixture of ethyl acetate-water (60 Ml, 4:1). Organic layer was washed with brine, dried (Na2SO4) and concentrated to yield the crude which upon silica gel column chromatographic purification (30% ethyl acetate-hexane) afforded 1.5 G of ethyl 4-(3-bromophenyl)-2-methylpyrimidine-5-carboxylate.

NMR (CDCl3): 9.05 (s, 1H), 7.74 (t, 1H), 7.62 (dd, 1H), 7.60 (dd, 1H), 7.4 (m, 1H), 4.24 (q, 2H), 2.83 (s, 3H), 1.17 (t, 3H)

### Part C: Preparation of 4-(3-bromophenyl)-2-methylpyrimidine-5-carbohydrazide

Hydrazine hydrate (1 Ml) was added to the solution of ethyl 4-(3-bromophenyl)-2-methylpyrimidine-5-carboxylate (1.5 G) in ethanol (10 Ml). The reaction mixture was refluxed for 16 h; cooled at room temperature and concentrated in vaccuo. The residue was partitioned between dichloromethane-water (50 Ml, 4:1). The organic layer was washed with water, dried and concentrated to yield 1.4 G of crude 4-(3-bromophenyl)-2-methylpyrimidine-5-carbohydrazide which was used as such in the next reaction.

### Part D: Preparation of 4-(3-bromophenyl)-2-methyl-5-(5-phenyl-1,3,4-oxadiazol-2-yl)pyrimidine

PyBroP (0.5 G, 0.9 mmol) was added to the solution of crude 4-(3-bromophenyl)-2-methylpyrimidine-5-carbohydrazide (0.2 G, 0.64 mmol) and N,N-diisopropylethylamine (0.3 Ml) in DMF (1 Ml). The reaction mixture was stirred at room temperature for 3 h. Diisopropyl carbodiimide (0.25 G, 0.98 mmol) was added to the reaction mixture and the mixture was heated to 190° in microwave reactor for 45 minutes. The reaction mixture was cooled and partitioned between ethyl acetate-water (50 Ml, 4:1). The organic layer was washed with brine, dried over Na2SO4 and concentrated. The crude was purified using silica gel column chromatography (20% ethyl acetate-hexane) to afford 170 mg of 4-(3-bromophenyl)-2-methyl-5-(5-phenyl-1,3,4-oxadiazol-2-yl)pyrimidine.

NMR (CDCl3): 9.13 (s, 1H), 8.13 (m, 3H), 7.7 (s,1H), 7.53 (m, 3H), 7.37 (m, 2H), 2.82 (s, 3H)

### Part E: Preparation of 3-(2-methyl-5-(5-phenyl-1,3,4-oxadiazol-2-yl)pyrimidin-4-yl)benzonitrile

Zinc cyanide (30 mg, 0.25 mmol) and ((C6H5)3P)4Pd (0) (10 mg, 0.02 mmol) was added to the solution of 4-(3-bromophenyl)-2-methyl-5-(5-phenyl-1,3,4-oxadiazol-2-yl)pyrimidine (82 mg, 0.2 mmol) in DMF (2 Ml). The reaction mixture was heated to 195° C. for 45 minutes in a microwave oven. The reaction mixture was cooled to room temperature and diluted with a mixture of ethyl acetate-water (25 Ml, 4:1). The organic layer was washed with brine, dried over Na2SO4 and concentrated in vaccuo to yield the crude which upon silica gel chromatographic purification yielded 62 mg of pure 3-(2-methyl-5-(5-phenyl-1,3,4-oxadiazol-2-yl)pyrimidin-4-yl)benzonitrile.

NMR (CDCl3): 9.13 (s, 1H), 8.32 (m, 1H), 8.13 (m, 2H), 7.7 (s,1H), 7.53 (m, 3H), 7.37 (m, 2H), 2.82 (s, 3H)

### Part F: Preparation of (3-(2-methyl-5-(5-phenyl-1,3,4-oxadiazol-2-yl)pyrimidin-4-yl)phenyl)methanamine

Sodium borohydride (20 mg, 0.54 mmol) was added to the suspension of 3-(2-methyl-5-(5-phenyl-1,3,4-oxadiazol-2-yl)pyrimidin-4-yl)benzonitrile (62 mg, 0.18 mmol) and nickel chloride hexahydrate (52 mg, 0.21 mmol) in ethanol (2 Ml). The reaction mixture was stirred at room temperature for 1 h and then concentrated in vaccuo. The residue was partitioned between dichloromethane-water (25 Ml, 4:1). The organic layer was washed with water, dried and concentrated to furnish 60 mg of crude (3-(2-methyl-5-(5-phenyl-1,3,4-oxadiazol-2-yl)pyrimidin-4-yl)phenyl)methanamine which was utilized as such in the next reaction.

MS m/z 344.15 M+H)

### Part G: Preparation of (2S)—N-(3-(2-methyl-5-(5-phenyl-1,3,4-oxadiazol-2yl)pyrimidin-4-yl)benzyl)-2-aminopropan-amide

Boc-S-alanine-OSu (30 mg) was added to the solution of crude (3-(2-methyl-5-(5-phenyl-1,3,4-oxadiazol-2-yl)pyrimidin-4-yl)phenyl)methanamine (60 mg) in dichloromethane (2 Ml). The reaction mixture was stirred at room temperature for 1 h. To this reaction mixture was added a trifluoroacetic acid in dichloromethane (2 Ml, 25% v/v). The resulting mixture was stirred for 45 minutes at room temperature and concentrated in vacuo. The crude product was purified using preparative HPLC using conditions below.
Conditions: Column—YMC ODS (20×50 mm)
Solvents—A—90% water—10% methanol—0.1% TFA
   B—10% water—90% methanol—0.1% TFA
Gradient—25% B to 100% B in 12 min
Retention time: 9.8 min

MS m/z 415.18 (M+H)

NMR (CD3OD): 9.2(s, 1H), 7.9 (m, 2H), 7.7 (s,1H), 7.53 (m, 3H), 7.37 (m, 2H), 7.07 (s, 1H), 4.56 (dd, 2H), 3.92 (m, 1H), 2.82 (s, 3H), 1.30 (d, 3H).

### (2S)—N-(3-(1-methyl-4-(5-phenyl-1,3,4-oxadiazol-2-yl)-1H-pyrazol-5-yl)benzyl)-2-aminopropanamide (Example 180)

### Example 180

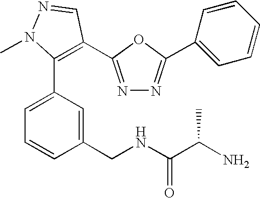

### Part A: Preparation of ethyl 5-(3-cyanophenyl)-1-methyl-1H-pyrazole-4-carboxylate

A mixture of ethyl-3-cyanobenzoylacetate (1.0 G, 5 mmol) and N,N-dimethylformamide dimethylacetal (2.74 G, 14 mmol) was heated to 100° C. for 24 h. The reaction mixture was concentrated in vacuo and the residue was re-dissolved in methanol (5 Ml), followed by addition of methyl hydrazine (0.3 Ml, 6 mmol). The reaction mixture was stirred at room temperature for 2 h and then concentrated in vaccuo. The residue was partitioned between ethyl acetate-water (60 Ml, 4:1), washed with water, dried over Na2SO4 and concentrated. The crude was purified using silica gel column chromatography (1 to 15% ethyl acetate-dichloromethane) to yield 0.36 G of required regioisomer; ethyl 5-(3-cyanophenyl)-1-methyl-1H-pyrazole-4-carboxylate and 0.3 G of ethyl 3-(3-cyanophenyl)-1-methyl-1H-pyrazole-4-carboxylate.

MS m/z 256.3(M+H)

### Part B: Preparation of ethyl 5-(3-(aminomethyl)phenyl)-1-methyl-1H-pyrazole-4-carboxylate

10% Pd on charcoal (100 mg) was added to the solution of ethyl 5-(3-cyanophenyl)-1-methyl-1H-pyrazole-4-carboxylate (100 mg, 3.9 mmol) and trifluoroacetic acid (50 Ul) in isopropanol. The reaction mixture was stirred under the blanket of hydrogen gas for 2 h. The reaction mixture was filtered over celite. The filtrate was concentrated in vaccuo to yield 79 mg of crude (˜85% by LCMS) ethyl 5-(3-(aminomethyl)phenyl)-1-methyl-1H-pyrazole-4-carboxylate which was carried as such to the next reaction.

### Part C: Preparation of ethyl 5-(3-(((S)-2-(tert-butoxycarbonyl)propanamido)methyl)phenyl)-1-methyl-1H-pyrazole-4-carboxylate

Boc-S-alanine-O-succinamide (87 mg, 0.3 mmol) was added to the solution of ethyl 5-(3-(aminomethyl)phenyl)-1-methyl-1H-pyrazole-4-carboxylate (78.2 mg, 0.3 mmol) and triethylamine (42 Ul) in dichloromethane (2 Ml). The solution was stirred at room temperature for 45 minutes. The reaction mixture was diluted with 10 Ml of dichloromethane and 5 mL of water. The organic layer was washed with water, dried over Na2SO4 and concentrated to yield the crude which was further purified by silica gel column chromatography (5% ethyl acetate-heptane) to afford 89 mg of ethyl 5-(3-(((S)-2-(tert-butoxycarbonyl)propanamido)methyl)phenyl)-1-methyl-1H-pyrazole-4-carboxylate

### Part D: Preparation of 5-(3-(((S)-2-(tert-butoxycarbonyl)propanamido)methyl)phenyl)-1-methyl-1H-pyrazole-4-carboxylic acid

Aqueous sodium hydroxide solution (0.2 Ml, 1 M) was added to the solution of ethyl 5-(3-(((S)-2-(tert-butoxycarbonyl)propanamido)methyl)phenyl)-1-methyl-1H-pyrazole-4-carboxylate (89 mg) in ethanol (3 Ml) and THF (1 Ml). The reaction mixture was heated to 60° C. for 6 h. The reaction was diluted with ethyl acetate-water (10 mL, 1:1). The organic layer was washed with 1 N NaOH. The combined aqueous layer was acidified with 1 N HCl and extracted with dichloromethane. Organic layer was washed with water, dried over Na2SO4 and concentrated to yield 50 mg of crude 5-(3-(((S)-2-(tert-butoxycarbonyl)propanamido)methyl)phenyl)-1-methyl-1H-pyrazole-4-carboxylic acid which was used as such in the next reaction.

MS m/z 403.1(M+H)

### Part E: tert-butyl (S)-1-(3-(1-methyl-4-(5-phenyl-1,3,4-oxadiazol-2-yl)-1H-pyrazol-5-yl)benzylamino)-1-oxopropan-2-ylcarbamate

Benzoic hydrazide (10.2 mg, 0.07 mmol) was added to the solution of 5-(3-(((S)-2-(tert-butoxycarbonyl)propanamido)methyl)phenyl)-1-methyl-1H-pyrazole-4-carboxylic acid (50 mg, 0.124 mmol), PyBroP (97 mg, 0.186 mmol) and Hunig's base (54.1 Ul, 0.3 mmol) in DMF (1 Ml). The reaction mixture was heated at 120° C. for 10 min in microwave. To this solution was added diisopropylcarbodiimide (100 Ul, 0.621 mmol) and the reaction mixture was further heated to 150° C. for 40 minutes in microwave. The reaction mixture was diluted with methanol (1 Ml) and the solution was directly loaded onto a preparative HPLC for purification to yield 25 mg of tert-butyl (S)-1-(3-(1-methyl-4-(5-phenyl-1,3,4-oxadiazol-2-yl)-1H-pyrazol-5-yl)benzylamino)-1-oxopropan-2-ylcarbamate.

The conditions for purification are as follows:
Conditions: Column—YMC ODS (20×50 mm)
Solvents—A—90% water—10% methanol—0.1% TFA
   B—10% water—90% methanol—0.1% TFA
Gradient—45% B to 100% B in 12 min
Retention time: 12 min

MS m/z 503.3 (M+H)

NMR (CDCl3): 8.6 (s, 1H), 8.12 (m, 2H), 7.74 (m, 1H), 7.54 (m, 2H), 7.38 (m, 1H), 7.2 (m, 1H), 7.06 (m, 1H), 6.6 (m, 2H), 4.58-4.67 (m, 2H), 4.3 (m, 1H), 3.7 (s, 3H), 1.5 (s, 9H), 1.3 (d, 3H)

### Part F: Preparation of (2S)—N-(3-(1-methyl-4-(5-phenyl-1,3,4-oxadiazol-2-yl)-1H-pyrazol-5-yl)benzyl)-2-aminopropanamide

tert-butyl (S)-1-(3-(1-methyl-4-(5-phenyl-1,3,4-oxadiazol-2-yl)-1H-pyrazol-5-yl)benzylamino)-1-oxopropan-2-ylcarbamate (25 mg) was treated with a mixture of trifluoroacetic acid in dichloromethane (0.2:1 Ml) at room temperature for 30 minutes. The reaction mixture was concentrated in vaccuo and the residue was re-dissolved in methanol (2 Ml). The methanolic solution was loaded onto a pre-washed and equilibrated (with methanol) cartridge containing 2 G of SCX (Waters). The column was eluted with 5 Ml OF METHANOL AND THEN WITH 10 Ml of 2 M ammonia solution in methanol. The later fractions were concentrated to yield 3 mg of (2S)—N-(3-(1-methyl-4-(5-phenyl-1,3,4-oxadiazol-2-yl)-1H-pyrazol-5-yl)benzyl)-2-aminopropanamide.

MS m/z 503.3 (M+H)

NMR (CD3OD): 8.3(s, 1H), 7.9 (m, 2H), 7.3-7.6 (m, 6H), 4.6(dd, 2H), 3.9 (m, 1H), 3.7 (s, 3H), 1.3 (d, 3H)

### (2S)—N-(3-(1-methyl-4-(5-phenyl-1,3,4-oxadiazol-2-yl)-1H-pyrazol-3-yl)benzyl)-2-aminopropanamide (Example 181)

### Example 181

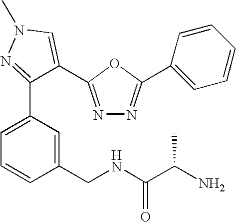

### Part A: Preparation of ethyl 3-(3-cyanophenyl)-1-methyl-1H-pyrazole-4-carboxylate

Obtained as a minor regioisomer during the preparation of 179 (Part A)

### Part B: Preparation of ethyl 3-(3-(aminomethyl)phenyl)-1-methyl-1H-pyrazole-4-carboxylate

10% Pd on charcoal (100 mg) was added to the solution of ethyl 3-(3-cyanophenyl)-1-methyl-1H-pyrazole-4-carboxylate

(120 mg, 3.9 mmol) and trifluoroacetic acid (50 Ul) in isopropanol. The reaction mixture was stirred under the blanket of hydrogen gas for 2 h. The reaction mixture was filtered over celite. The filtrate was concentrated in vaccuo to yield 79 mg of crude (˜85% by LCMS) ethyl 3-(3-(aminomethyl)phenyl)-1-methyl-1H-pyrazole-4-carboxylate which was carried to the next step without further purification.

MS m/z 261.2 (M+H)

### Part C: Preparation of ethyl 3-(3-(((S)-2-(tert-butoxycarbonyl)propanamido)methyl)phenyl)-1-methyl-1H-pyrazole-4-carboxylate

Boc-S-alanine-O-succinamide (87 mg, 0.3 mmol) was added to the solution of ethyl 3-(3-(aminomethyl)phenyl)-1-methyl-1H-pyrazole-4-carboxylate (79 mg, 0.3 mmol) and triethylamine (42 Ul) in dichloromethane (2 Ml). The solution was stirred at room temperature for 45 minutes. The reaction mixture was diluted with 10 Ml of dichloromethane and 5 mL of water. The organic layer was washed with water, dried over Na2SO4 and concentrated to yield the crude which was further purified by silica gel column chromatography (5% ethyl acetate-heptane) to afford 89 mg of ethyl 5-(3-(((S)-2-(tert-butoxycarbonyl)propanamido)methyl)phenyl)-1-methyl-1H-pyrazole-4-carboxylate.

MS m/z 431.3(M+H)

### Part D: 3-(3-(((S)-2-(tert-butoxycarbonyl)propanamido)methyl)phenyl)-1-methyl-1H-pyrazole-4-carboxylic acid hydrazide

Hydrazine monohydrate (23 uL) was added to the solution of ethyl 5-(3-(((S)-2-(tert-butoxycarbonyl)propanamido)methyl)phenyl)-1-methyl-1H-pyrazole-4-carboxylate (77 mg, 0.18 mmol) in ethanol. The solution was heated for 5 days at 60° C. and then cooled to room temperature and concentrated in vaccuo. The residue was partitioned between water and dichloromethane (25 Ml, 1:4). The organic layer was washed with water, dried and concentrated to yield 46 mg of 3-(3-(((S)-2-(tert-butoxycarbonyl)propanamido)methyl)phenyl)-1-methyl-1H-pyrazole-4-carboxylic acid hydrazide.

MS m/z 417(M+H)

### Part E: Preparation of tert-butyl (S)-1-(3-(1-methyl-4-(5-phenyl-1,3,4-oxadiazol-2-yl)-1H-pyrazol-3-yl)benzylamino)-1-oxopropan-2-ylcarbamate

PyBrOP (84.5 mg, 0.17 mmol) was added to the solution of 3-(3-(((S)-2-(tert-butoxycarbonyl)propanamido)methyl)phenyl)-1-methyl-1H-pyrazole-4-carboxylic acid hydrazide (45 mg, 0.11 mmol), benzoic acid (20 mg, 0.17 mmol) and Hunig base (47 Ul, 0.27 mmol) in DMF (1 Ml). The reaction mixture was heated at 120° C. in microwave for 10 minutes. The reaction mixture was cooled to room temperature and di-isopropylcarbodiimide (85 mg, 0.54) was added. The reaction mixture was heated at 150° C. for 30 minutes in microwave oven. The reaction was cooled to room temperature and the crude was purified using preparative HPLC using the conditions below to afford 5.2 mg of tert-butyl (S)-1-(3-(1-methyl-4-(5-phenyl-1,3,4-oxadiazol-2-yl)-1H-pyrazol-3-yl)benzylamino)-1-oxopropan-2-ylcarbamate.
Conditions: Column—YMC ODS (20×50 mm)
Solvents—A—90% water—10% methanol—0.1% TFA
   B—10% water—90% methanol—0.1% TFA
Gradient—45% B to 100% B in 12 min
Retention time: 12 min

MS m/z 503.3 (M+H)

NMR (CDCl3): 8.6 (s, 1H), 8.12 (m, 2H), 7.74 (m, 1H), 7.54 (m, 2H), 7.38 (m, 1H), 7.2 (m, 1H), 7.06 (m, 1H), 6.6 (m, 2H), 4.58-4.67 (m, 2H), 4.3 (m, 1H), 3.7 (s, 3H), 1.5 (s, 9H), 1.3 (d, 3H)

### Part F: Preparation of (2S)—N-(3-(1-methyl-4-(5-phenyl-1,3,4-oxadiazol-2-yl)-1H-pyrazol-3-yl)benzyl)-2-aminopropanamide

tert-butyl (S)-1-(3-(1-methyl-4-(5-phenyl-1,3,4-oxadiazol-2-yl)-1H-pyrazol-3-yl)benzylamino)-1-oxopropan-2-ylcarbamate was treated with a mixture of trifluoroacetic acid in dichloromethane (0.2:1 Ml) at room temperature for 30 minutes. The reaction mixture was concentrated in vaccuo and the residue was re-dissolved in methanol (2 Ml). The methanolic solution was loaded onto a pre-washed and equilibrated (with methanol) cartridge containing 2 G of SCX (Waters). The column was eluted with 5 Ml OF METHANOL AND THEN WITH 10 Ml of 2 M ammonia solution in methanol. The later fractions were concentrated to yield 3 mg of (2S)—N-(3-(1-methyl-4-(5-phenyl-1,3,4-oxadiazol-2-yl)-1H-pyrazol-5-yl)benzyl)-2-aminopropanamide.

MS m/z 503.3 (M+H)

NMR (CD3OD): 8.3(s, 1H), 7.9 (m, 2H), 7.3-7.6 (m, 6H), 4.6(dd, 2H), 3.9 (m, 1H), 3.7 (s, 3H), 1.3 (d, 3H)

### (2S)—N-(3-(5-(5-phenyl-1,3,4-oxadiazol-2-yl)-1H-imidazol-1-yl)benzyl)-2-aminopropanamide (Example 182)

### Example 182

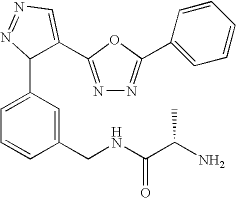

### Part A: Preparation of methyl 1-(3-cyanophenyl)-1H-imidazole-5-carboxylate

Using the procedure as described in example #191 in U.S. Pat. No. 6,548,512

### Part B: Preparation of methyl 1-(3-(aminomethyl)phenyl)-1H-imidazole-5-carboxylate

10% Pd on charcoal (200 mg) and platinum oxide (100 mg) were added to the solution of methyl 1-(3-cyanophenyl)-1H-imidazole-5-carboxylate (300 mg, 1.32 mmol) and trifluoroacetic acid (0.1 mL) in ethanol. The reaction mixture was stirred under the blanket of hydrogen gas for 24 h. The reaction mixture was filtered over celite. The filtrate was concentrated in vaccuo to give the crude. The crude was purified using preparative HPLC using conditions below to yield 100 mg of methyl 1-(3-(aminomethyl)phenyl)-1H-imidazole-5-carboxylate
Conditions: Column—YMC ODS (20×50 mm)
Solvents—A—90% water—10% methanol—0.1% TFA
   B—10% water—90% methanol—0.1% TFA
Gradient—25% B to 100% B in 12 min
Retention time: 9.6 min

MS m/z 232.2 (M+H)

### Part C: Preparation of methyl 1-(3-(((S)-2-(tert-butoxycarbonyl)propanamido)methyl)phenyl)-1H-imidazole-5-carboxylate

Boc-S-alanine-O-succinamide (125 mg, 0.43 mmol) was added to the solution of methyl 1-(3-(aminomethyl)phenyl)-1H-imidazole-5-carboxylate (101 mg, 0.43 mmol) and triethylamine (60 Ul) in dichloromethane (2 Ml). The solution was stirred at room temperature for 45 minutes. The reaction mixture was diluted with 10 Ml of dichloromethane and 5 mL of water. The organic layer was washed with water, dried over Na2SO4 and concentrated to yield the crude which was further purified by silica gel column chromatography (5% ethyl acetate-heptane) to afford 89 mg of ethyl 5-(3-(((S)-2-(tert-butoxycarbonyl)propanamido)methyl)phenyl)-1-methyl-1H-pyrazole-4-carboxylate.

NMR (CDCl3): 7.6 (s, 1H), 7.3-7.5 (m, 3H), 7.2 (m, 1H), 7.06 (s, 1H), 6.1 (m, 2H), 4.67 (m, 2H), 4.1 (m, 1H), 3.7 (s, 3H), 1.3-1.5 (m, 12H)

MS m/z 431.3(M+H)

### Part D: Preparation of 1-(3-(((S)-2-(tert-butoxycarbonyl)propanamido)methyl)phenyl)-1H-imidazole-5-carboxylic acid hydrazide

Hydrazine monohydrate (14 uL) was added to the solution of methyl 1-(3-(((S)-2-(tert-butoxycarbonyl)propanamido) methyl)phenyl)-1H-imidazole-5-carboxylate (46 mg, 0.113 mmol) in ethanol (5 Ml). The solution was heated for 24 h at 60° C. and then cooled to room temperature and concentrated in vaccuo. The residue was partitioned between water and dichloromethane (25 Ml, 1:4). The organic layer was washed with water, dried and concentrated to yield 32 mg of 1-(3-(((S)-2-(tert-butoxycarbonyl)propanamido) methyl)phenyl)-1H-imidazole-5-carboxylic acid hydrazide.

MS m/z 403(M+H)

### Part E: Preparation of tert-butyl (S)-1-(3-(5-(5-phenyl-1,3,4-oxadiazol-2-yl)-1H-imidazol-1-yl)benzylamino)-1-oxopropan-2-ylcarbamate

PyBrOP (62.5 mg, 0.12 mmol) was added to the solution of 1-(3-(((S)-2-(tert-butoxycarbonyl)propanamido)methyl)phenyl)-1H-imidazole-5-carboxylic acid hydrazide (32 mg, 0.08 mmol), benzoic acid (10 mg, 0.08 mmol) and Hunig base (35 Ul, 0.2 mmol) in DMF (1 Ml). The reaction mixture was heated at 120° C. in microwave for 15 minutes. The reaction mixture was cooled to room temperature and di-isopropylcarbodiimide (62 mg, 0.40) was added. The reaction mixture was heated at 150° C. for 30 minutes in microwave oven. The reaction was cooled to room temperature and the crude was purified using preparative HPLC using the conditions below to afford 40 mg of tert-butyl (S)-1-(3-(5-(5-phenyl-1,3,4-oxadiazol-2-yl)-1H-imidazol-1-yl)benzylamino)-1-oxopropan-2-ylcarbamate.
Conditions: Column—YMC ODS (20×50 mm)
Solvents—A—90% water—10% methanol—0.1% TFA
   B—10% water—90% methanol—0.1% TFA
Gradient—45% B to 100% B in 12 min
Retention time: 10 min

MS m/z 489 (M+H)

NMR (CDCl3): 8.6 (s, 1H), 8.12 (m, 2H), 7.74 (m, 1H), 7.54 (m, 2H), 7.38 (m, 1H), 7.2 (m, 1H), 7.06 (m, 1H), 6.6 (m, 2H), 4.58-4.67 (m, 2H), 4.3 (m, 1H), 3.7 (s, 3H), 1.5 (s, 9H), 1.3 (d, 3H)

### Part F: Preparation of (2S)—N-(3-(5-(5-phenyl-1,3,4-oxadiazol-2-yl)-1H-imidazol-1-yl)benzyl)-2-aminopropanamide trifluoroacetate salt

39 mg (0.08 mmol) of tert-butyl (S)-1-(3-(5-(5-phenyl-1,3,4-oxadiazol-2-yl)-1H-imidazol-1-yl)benzylamino)-1-oxopropan-2-ylcarbamate in dichloromethane (2 Ml) was treated trifluoroacetic acid (0.4 Ml). The reaction mixture was stirred at room temperature for 45 minutes and then concentrated in vaccuo to afford 20 mg of (2S)—N-(3-(5-(5-phenyl-1,3,4-oxadiazol-2-yl)-1H-imidazol-1-yl)benzyl)-2-aminopropanamide trifluoroacetate salt.

MS m/z 389 (M+H)

NMR (CD3OD): 8.4 (s, 1H), 8.12 (m, 1H), 7.92 (d, 2H), 7.54 (m, 5H), 4.50(dd, 2H), 3.95 (m, 1H), 1.3 (d, 3H)

### (2S)—N-(3-(4-(5-phenyl-1,3,4-oxadiazol-2-yl)oxazol-5-yl)benzyl)-2-aminopropanamide (Example 183)

### Example 183

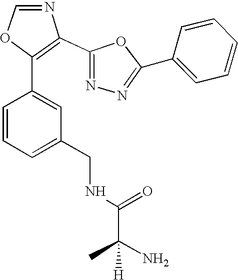

### Part A: Preparation of methyl 5-(3-cyanophenyl)oxazole-4-carboxylate

Methyl isocyanoacetate (0.5 Ml, 6 mmol) was added to the solution of 3-cyanobenzoyl chloride (1 G, 6 mmol) and triethylamine (2.5 Ml) in dichloromethane (20 ml). The reaction mixture was stirred for 24 h. The reaction mixture was diluted with 20 mL of dichloromethane and 10 mL of water. The organic layer was washed with water, dried over Na2SO4 and concentrated. The crude product was recrystallized from hot methanol to give 0.98 G of methyl 5-(3-cyanophenyl)oxazole-4-carboxylate

NMR (CDCl3):8.7 (s, 1H), 8.2 (d, 1H), 7.93 (s, 1H), 7.82 (d, 1H), 7.6 (m, 1H), 3.78 (s, 3H)

### Part B: Preparation of methyl 5-(3-(aminomethyl)phenyl)oxazole-4-carboxylate

10% Pd on charcoal (100 mg) and platinum oxide (170 mg) were added to the solution of methyl 1-(3-cyanophenyl)-1H-imidazole-5-carboxylate (300 mg, 1.32 mmol) and trifluoroacetic acid (50 uL) in isopropanol. The reaction mixture was stirred under the blanket of hydrogen gas for 4 h. The reaction mixture was filtered over celite. The filtrate was concentrated in vaccuo to give the crude. The crude was dissolved in methanol and loaded over a SCX column (2 G) pre-equilibrated with methanol. The column was eluted with 10 Ml of methanol and then 10 mL of 2N methanolic ammonia solution. The fractions from the later elution were combined and concentrated to yield 123 mg of methyl 5-(3-(aminomethyl)phenyl)oxazole-4-carboxylate.

MS m/z 234 (M+H)

### Part C: Preparation of methyl 5-(3-(((S)-2-(tert-butoxycarbonyl)propanamido)methyl)phenyl)oxazole-4-carboxylate

Boc-S-alanine-O-succinamide (153 mg, 0.53 mmol) was added to the solution of methyl 5-(3-(aminomethyl)phenyl)oxazole-4-carboxylate (123 mg, 0.53 mmol) and triethylamine (75 uL) in dichloromethane (2 mL). The solution was stirred at room temperature for 45 minutes. The reaction mixture was diluted with 10 Ml of dichloromethane and 5 mL of water. The organic layer was washed with water, dried over Na2SO4 and concentrated to yield the crude which was further purified by silica gel column chromatography (5% ethyl acetate-heptane) to afford 106 mg of methyl 5-(3-(((S)-2-(tert-butoxycarbonyl)propanamido)methyl)phenyl)oxazole-4-carboxylate

NMR (CDCl3): 8.7 (s, 1H), 7.8 (d, 1H), 7.35 (m, 2H), 7.15 (d, 1H), 6.1 (m, 1H), 4.7 (s, 2H), 4.1 (m, 1H), 3.8 (s, 3H), 1.4 (m, 12H)

MS m/z 404(M+H)

### Part D: Preparation of 5-(3-(((S)-2-(tert-butoxycarbonyl)propanamido)methyl)phenyl)oxazole-4-carboxylic acid hydrazide

Hydrazine monohydrate (30 uL) was added to the solution of methyl 5-(3-(((S)-2-(tert-butoxycarbonyl)propanamido) methyl)phenyl)oxazole-4-carboxylate (106 mg, 0.26 mmol) in ethanol (5 mL). The solution was heated for 1 h at 60° C. and then cooled to room temperature and concentrated in vaccuo. The residue was partitioned between water and dichloromethane (25 mL, 1:4). The organic layer was washed with water, dried over Na2SO4 and concentrated to yield 91 mg of 5-(3-(((S)-2-(tert-butoxycarbonyl)propanamido) methyl)phenyl)oxazole-4-carboxylic acid hydrazide.

MS m/z 404(M+H)

### Part E: Preparation of tert-butyl (S)-1-(3-(4-(5-phenyl-1,3,4-oxadiazol-2-yl)oxazol-5-yl)benzylamino)-1-oxopropan-2-ylcarbamate

PyBrOP (174 mg, 0.336 mmol) was added to the solution of 5-(3-(((S)-2-(tert-butoxycarbonyl)propanamido) methyl)phenyl)oxazole-4-carboxylic acid hydrazide (90 mg, 0.224 mmol), benzoic acid (41 mg, 0.336 mmol) and Hunig base (98 uL, 0.56 mmol) in DMF (1 Ml). The reaction mixture was heated at 120° C. in microwave for 15 minutes. The reaction mixture was cooled to room temperature and di-isopropylcarbodiimide (141 mg, 1.12) was added. The reaction mixture was heated at 150° C. for 30 minutes in microwave oven. The reaction was cooled to room temperature and the crude was purified using preparative HPLC using the conditions below to afford 40 mg of tert-butyl (S)-1-(3-(4-(5-phenyl-1,3,4-oxadiazol-2-yl)oxazol-5-yl)benzylamino)-1-oxopropan-2-ylcarbamate.
Conditions: Column—YMC ODS (20×50 mm)
Solvents—A—90% water—10% methanol—0.1% TFA
   B—10% water—90% methanol—0.1% TFA
Gradient—45% B to 100% B in 12 min
Retention time: 10 min

NMR (CDCl3):8.21 (s, 1H), 8.1 (m, 2H), 7.7 (d, 1H), 7.5 (m, 3H), 7.33 (m, 2H), 7.0 (d, 1H), 6.8 (bs, 1H), 4.63 (dd, 2H), 4.32 (m, 1H), 1.5 (s, 9H), 1.32 (d, 3H)

MS m/z 490 (M+H)

### Part F: Preparation of (2S)—N-(3-(4-(5-phenyl-1,3,4-oxadiazol-2-yl)oxazol-5-yl)benzyl)-2-aminopropanamide

Trifluoroacetic acid (1 Ml) was added to the solution of tert-butyl (S)-1-(3-(4-(5-phenyl-1,3,4-oxadiazol-2-yl)oxazol-5-yl)benzylamino)-1-oxopropan-2-ylcarbamate (32 mg) in dichloromethane (3 Ml). The reaction mixture was stirred for 45 minutes at room temperature and then concentrated in vaccuo. The product was dissolved in 2 mL of methanol and loaded onto pre-equilibrated (with methanol) column of SCX. The column was eluted with methanol (2×2 mL) and then with 2 N solution of ammonia in methanol. The fractions were concentrated to yield 16 mg of (2S)—N-(3-(4-(5-phenyl-1,3,4-oxadiazol-2-yl)oxazol-5-yl)benzyl)-2-aminopropanamide.

MS m/z 390.3 (M+H)

NMR (CD3OD): 8.6 (s, 1H), 8.4 (s, 1H), 8.2 (m, 3H), 7.6 (m, 7H), 4.5 (dd, 2H), 3.6 (m, 1H), 1.32 (d, 3H)

### (2S)—N-(3-(5-methyl-4-(5-phenyl-1,3,4-oxadiazol-2-yl)isoxazol-3-yl)benzyl)-2-aminopropanamide (Example 184)

### Example 184

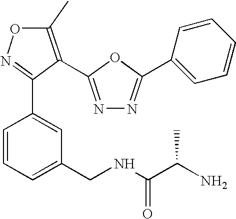

### Part A: Preparation of 3-(3-(aminomethyl)phenyl)-5-methylisoxazole-4-carboxylic acid

Using the procedure as reported in WO 9828282

### Part B: Preparation of 3-(3-(((S)-2-(tert-butoxycarbonyl)propanamido)methyl)phenyl)-5-methylisoxazole-4-carboxylic acid

Boc-S-alanine-O-succinamide (94 mg, 0.33 mmol) was added to the solution of 3-(3-(aminomethyl)phenyl)-5-methylisoxazole-4-carboxylic acid (70 mg, 0.3 mmol) and triethylamine (75 uL) in dichloromethane (2 mL). The solution was stirred at room temperature for 45 minutes. The reaction mixture was diluted with 10 Ml of dichloromethane and 5 mL of water. The organic layer was washed with water, dried over Na2SO4 and concentrated to yield the crude which was further purified by silica gel column chromatography (5% ethyl acetate-heptane) to afford 120 mg of 3-(3-(((S)-2-(tert-butoxycarbonyl)propanamido)methyl)phenyl)-5-methylisoxazole-4-carboxylic acid

NMR (CDCl3): 11 (s, 1H), 7.2-7.3 (m, 3H), 7.15 (d, 1H), 4.5 (s, 2H), 3.4 (m, 1H), 1.4 (m, 12H),

MS m/z 404(M+H)

### Part B: Preparation of tert-butyl (S)-1-(3-(5-methyl-4-(5-phenyl-1,3,4-oxadiazol-2-yl)isoxazol-3-yl)benzylamino)-1-oxopropan-2-ylcarbamate

PyBroP (44 mg, 0.08 mmol) was added to the solution of 3-(3-(((S)-2-(tert-butoxycarbonyl)propanamido)methyl)phenyl)-5-methylisoxazole-4-carboxylic acid (28 mg, 0.07 mmol) and N,N-diisopropylethylamine (18 mg, 0.14 mmol) in DMF (1 Ml). The reaction mixture was stirred for 16 h at room temperature and then di-isopropylcarbodiimide (36 mg, 0.28 mmol) was added to the reaction mixture. The reaction mixture was heated at 180° C. in a microwave oven for 30 minutes. The reaction mixture was diluted with 1 mL of methanol and the crude was purified using preparative HPLC using the conditions as below to yield 7 mg of tert-butyl (S)-1-(3-(5-methyl-4-(5-phenyl-1,3,4-oxadiazol-2-yl)isoxazol-3-yl)benzylamino)-1-oxopropan-2-ylcarbamate
Conditions: Column—YMC ODS (20×50 mm)
Solvents—A—90% water—10% methanol—0.1% TFA
   B—10% water—90% methanol—0.1% TFA
Gradient—45% B to 100% B in 12 min
Retention time: 9.5 min

MS m/z 504 (M+H)

### Part C: Preparation of (2S)—N-(3-(5-methyl-4-(5-phenyl-1,3,4-oxadiazol-2-yl)isoxazol-3-yl)benzyl)-2-aminopropanamide

7 mg of tert-butyl (S)-1-(3-(5-methyl-4-(5-phenyl-1,3,4-oxadiazol-2-yl)isoxazol-3-yl)benzylamino)-1-oxopropan-2-ylcarbamate was treated with 0.1 mL of trifluoroacetic acid in 0.5 mL dichloromethane. The reaction mixture was stirred at room temperature for 30 minutes and then evaporated in vaccuo to yield 6 mg of trifluoroacetic acid salt of (2S)—N-(3-(5-methyl-4-(5-phenyl-1,3,4-oxadiazol-2-yl)isoxazol-3-yl)benzyl)-2-aminopropanamide.

NMR (CD3OD): 7.89 (m, 2H), 7.74 (s, 1H), 7.66-7.51 (m, 6H), 4.51 (m, 2H), 3.98 (q, 1H), 2.87 (s, 3H), 1.51 (d, 3H)

MS m/z 404 (M+H)

### N-(3-(3-methyl-5-(5-phenyl-1,3,4-oxadiazol-2-yl)-1H-pyrazol-1-yl)benzyl)-2-aminopropanamide (Example 185)

### Example 185

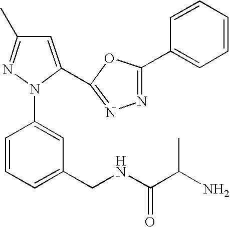

### Part A: Preparation of 1-(3-(aminomethyl)phenyl)-3-methyl-1H-pyrazole-5-carboxylic acid hydrate

Using the procedure as reported (Pinto et al, J Med Chem, 566-578 (2001)).

### Part B: Preparation of 1-(3-(((S)-2-(tert-butoxycarbonyl)propanamido)methyl)phenyl)-3-methyl-1H-pyrazole-5-carboxylic acid

Boc-S-alanine-O-succinamide (94 mg, 0.33 mmol) was added to the solution of 3-(3-(aminomethyl)phenyl)-5-methylisoxazole-4-carboxylic acid (70 mg, 0.3 mmol) and triethylamine (152 uL) in dichloromethane (2 mL). The solution was stirred at room temperature for 45 minutes. The reaction mixture was diluted with 10 mL of dichloromethane and 5 mL of water. The organic layer was washed with water, dried over Na2SO4 and concentrated to yield the crude which was further purified by silica gel column chromatography (5% ethyl acetate-heptane) to afford 120 mg of 3-(3-(((S)-2-(tert-butoxycarbonyl)propanamido)methyl)phenyl)-5-methylisoxazole-4-carboxylic acid

NMR (CDCl3): 11 (s, 1H), 7.2-7.3 (m, 3H), 7.15 (d, 1H), 4.5 (s, 2H), 3.4 (m, 1H), 1.4 (m, 12H),

MS m/z 404(M+H)

### Part C: Preparation of N-(3-(3-methyl-5-(5-phenyl-1,3,4-oxadiazol-2-yl)-1H-pyrazol-1-yl)benzyl)-2-aminopropanamide (TFA salt)

PyBroP (44 mg, 0.084 mmol) was added to the solution of 3-(3-(((S)-2-(tert-butoxycarbonyl)propanamido)methyl)phenyl)-5-methylisoxazole-4-carboxylic acid (28 mg, 0.07 mmol), benzoic hydrazide (11.4 mg, 0.084 mmol) and N,N-diisopropylethylamine (25 Ul, 0.14 mmol) in DMF (0.5 Ml). The reaction mixture was stirred at room temperature for 2 h. Diisopropylcarbodiimide (36 mg, 0.28 mmol) was added to the reaction mixture and heated in a microwave oven at 185° C. for 30 minutes. The reaction mixture was directly loaded onto a preparative HPLC column equilibrated with 25% solvent B (as defined below) and purified using the conditions below:
Conditions: Column—YMC ODS (20×50 mm)
Solvents—A—90% water—10% methanol—0.1% TFA
   B—10% water—90% methanol—0.1% TFA
Gradient—25% B to 100% B in 12 min
Retention time: 10 min

MS m/z 503.3 (M+H)

The product was taken up in a mixture of dichloromethane and trifluoroacetic acid (0.8:0.2 Ml) and stirred at room temperature for 30 minutes. The solvent was evaporated in vaccuo to afford 7.6 mg of trifluoroacetic acid salt of N-(3-(3-methyl-5-(5-phenyl-1,3,4-oxadiazol-2-yl)-1H-pyrazol-1-yl)benzyl)-2-aminopropanamide

NMR(CD₃OD) 7.92 (m, 2H), 7.65-7.45 (m, 7H), 7.12 (s, 1H), 4.53 (m, 2H), 3.97 (q, J=7.1 Hz, 1H), 2.43 (s, 3H), 1.51 (d, J=7.1 Hz, 3H).

MS m/z 517.3 (M+H)

### (2S)-2-amino-N-((2-methyl-5-(7-oxo-6-(4-(2-oxopiperidin-1-yl)phenyl)-3-(trifluoromethyl)-4,5,6,7-tetrahydropyrazolo[3,4-c]pyridin-1-yl)phenyl)methyl)propanamide (Example 186)

### Example 186

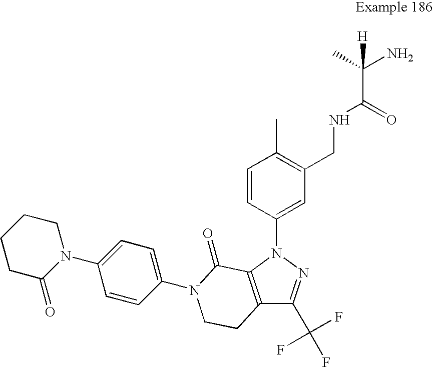

### Part A: Preparation of 1-(3-(aminomethyl)-4-methylphenyl)-6-(4-(2-oxopiperidin-1-yl)phenyl)-3-(trifluoromethyl)-5,6-dihydro-1H-pyrazolo[3,4-c]pyridin-7(4H)-one

Using the same procedure as reported in WO2003049681

### Part B: Preparation of 1-(3-(aminomethyl)-4-methylphenyl)-6-(4-(2-oxopiperidin-1-yl)phenyl)-3-(trifluoromethyl)-5,6-dihydro-1H-pyrazolo[3,4-c]pyridin-7(4H)-one

Boc-S-alanine-O-succinamide (31 mg, 0.11 mmol) was added to the solution of 1-(3-(aminomethyl)-4-methylphenyl)-6-(4-(2-oxopiperidin-1-yl)phenyl)-3-(trifluoromethyl)-5,6-dihydro-1H-pyrazolo[3,4-c]pyridin-7(4H)-one (30 mg, 0.1 mmol) and triethylamine (75 uL) in dichloromethane (2 mL). The solution was stirred at room temperature for 45 minutes. The reaction mixture was diluted with 10 Ml of dichloromethane and 5 mL of water. The organic layer was washed with water, dried over Na2SO4 and concentrated to yield the crude.

The crude was treated with a mixture of trifluoroacetic acid and dichloromethane (0.2:1 Ml) for 45 minutes. The reaction mixture was concentrated to give the crude which was purified using preparative HPLC using the conditions outlined below to afford 8.5 mg of trifluoroacetic acid salt of 1-(3-(aminomethyl)-4-methylphenyl)-6-(4-(2-oxopiperidin-1-yl)phenyl)-3-(trifluoromethyl)-5,6-dihydro-1H-pyrazolo[3,4-c]pyridin-7(4H)-one
Conditions: Column—YMC ODS (20×50 mm)
Solvents—A—90% water—10% methanol—0.1% TFA
   B—10% water—90% methanol—0.1% TFA
Gradient—25% B to 100% B in 12 min
Retention time: 8.8 min

MS m/z 569.5(M+H)

### Benzyl 1-(3-(((S)-2-aminopropanamido)methyl)phenyl)-3-(trifluoromethyl)-1H-pyrazol-5-ylcarbamate (Example 187)

### Example 187

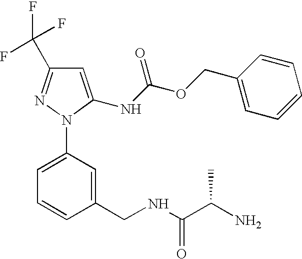

### Part A: Preparation of benzyl 1-(3-(((S)-2-(tert-butoxycarbonyl)aminopropanamido)methyl)phenyl)-3-(trifluoromethyl)-1H-pyrazol-5-ylcarbamate

Diphenylphosphoryl azide (19 mg, 0.07 mmol) was added to the solution of 1-(3-(((S)-2-(tert-butoxycarbonyl)propanamido)methyl)phenyl)-3-(trifluoromethyl)-1H-pyrazole-5-carboxylic acid (23 mg, 0.05 mmol) and triethylamine (10 mg, 0.1 mmol) in 1,4-dioxane (0.6 Ml). The reaction mixture was stirred at room temperature for 16 h. Benzyl alcohol (22 mg, 0.2 mmol) was added to the reaction and the mixture was heated at 80° C. for 1 h. The reaction mixture was concentrated and the crude was purified using silica gel column chromatography (1% MeOH—CH₂Cl₂) to afford 22 mg of benzyl 1-(3-(((S)-2-(tert-butoxycarbonyl)aminopropanamido)methyl)phenyl)-3-(trifluoromethyl)-1H-pyrazol-5-ylcarbamate

MS m/z 562.6 (M+H)

### Part B: Preparation of benzyl 1-(3-(((S)-2-aminopropanamido)methyl)phenyl)-3-(trifluoromethyl)-1H-pyrazol-5-ylcarbamate

Benzyl 1-(3-(((S)-2-(tert butoxycarbonyl)aminopropanamido) methyl)phenyl)-3-(trifluoromethyl)-1H-pyrazol-5-ylcarbamate (9 mg, 0.016 mmol) was treated with a mixture of trifluoroacetic acid in dichloromethane (0.1:0.3 Ml). The reaction mixture was stirred at room temperature for 45 minutes and then concentrated in vaccuo to yield 9 mg of trifluoroacetic acid salt of benzyl 1-(3-(((S)-2-(tert-butoxycarbonyl)aminopropanamido) methyl)phenyl)-3-(trifluoromethyl)-1H-pyrazol-5-ylcarbamate.

NMR (CD3OD): 7.51-7.26 (m, 9H), 6.72 (s, 1H), 5.13 (s, 2H), 4.46 (m, 2H), 3.92 (q, J=7.1 Hz, 1H), 1.51 (d, J=7.1 Hz, 3H).

MS m/z 462 (M+H)

### (2S)—N-(3-(5-(2-phenylacetamido)-3-(trifluoromethyl)-1H-pyrazol-1-yl)benzyl)-2-aminopropanamide (Example 188)

### Example 188

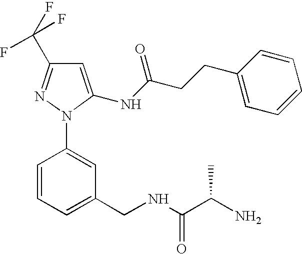

### Part A: Preparation of tert-butyl (S)-1-(3-(5-amino-3-(trifluoromethyl)-1H-pyrazol-1-yl)benzylamino)-1-oxopropan-2-ylcarbamate

10% Palladium on carbon (6 mg) was added to the solution of benzyl 1-(3-(((S)-2-(tert-butoxycarbonyl)aminopropanamido) methyl)phenyl)-3-(trifluoromethyl)-1H-pyrazol-5-ylcarbamate (9 mg, 0.016 mmol) in methanol (1 Ml). The suspension was stirred under the blanket of hydrogen gas (using balloon) for 4 h. The reaction mixture was filtered over a pad of celite and the celite was washed with methanol. The combined methanol layer was concentrated in vaccuo to afford 6.8 mg of tert-butyl (S)-1-(3-(5-amino-3-(trifluoromethyl)-1H-pyrazol-1-yl)benzylamino)-1-oxopropan-2-ylcarbamate.

MS m/z 428.3 (M+H)

### Part B: Preparation of tert-butyl (S)-1-(3-(5-(2-phenylacetamido)-3-(trifluoromethyl)-1H-pyrazol-1-yl)benzylamino)-1-oxopropan-2-ylcarbamate

2-phenylacetyl chloride (5 mg, 0.03 mmol) was added to the solution of tert-butyl (S)-1-(3-(5-amino-3-(trifluoromethyl)-1H-pyrazol-1-yl)benzylamino)-1-oxopropan-2-ylcarbamate (6 mg, 0.014 mmol) and triethylamine (7 mg, 0.07 mmol) in dichloromethane (1 Ml). The reaction mixture was stirred at room temperature for 16 h. The reaction was concentrated in vaccuo and the crude product was re-dissolved in 1 mL of methanol and loaded onto preparative HPLC column and purified using conditions as shown below.
Conditions: Column—YMC ODS (20×50 mm)
Solvents—A—90% water—10% methanol—0.1% TFA
   B—10% water—90% methanol—0.1% TFA
Gradient—25% B to 100% B in 12 min
Retention time: 9.2 min

MS m/z 546.5 (M+H)

### Part B: Preparation of (2S)—N-(3-(5-(2-phenylacetamido)-3-(trifluoromethyl)-1H-pyrazol-1-yl)benzyl)-2-aminopropanamide trifluoroacetic acid salt

tert-Butyl (S)-1-(3-(5-(2-phenylacetamido)-3-(trifluoromethyl)-1H-pyrazol-1-yl)benzylamino)-1-oxopropan-2-ylcarbamate (3 mg) in dichloromethane (0.2 mL) was treated with a trifluoroacetic acid (50 Ul). The reaction mixture was stirred at room temperature for 30 minutes. The reaction mixture was concentrated in vaccuo to furnish 2 mg of trifluoroacetate salt of (2S)—N-(3-(5-(2-phenylacetamido)-3-(trifluoromethyl)-1H-pyrazol-1-yl)benzyl)-2-aminopropanamide

NMR (CD3OD): 7.43-7.22 (m, 9H), 6.77 (s, 1H), 4.44 (m, 2H), 3.92 (q, J=7.1 Hz, 1H), 3.62 (s, 1H), 1.51 (d, J=7.1 Hz, 3H).

MS m/z 446 (M+H)

### 1-(1-(3-(((S)-2-aminopropanamido)methyl)phenyl)-3-(trifluoromethyl)-1H-pyrazol-5-yl)-3-phenylurea (Example 189)

### Example 189

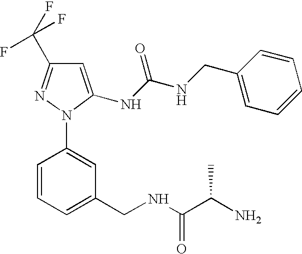

Phenyl isocyanate (6 mg, 0.05 mmol) was added to the solution of tert-butyl (S)-1-(3-(5-amino-3-(trifluoromethyl)-1H-pyrazol-1-yl)benzylamino)-1-oxopropan-2-ylcarbamate (13 mg, 0.03 mmol) and triethylamine (10 mg, 0.1 mmol) in dichloromethane (0.5 mL). The reaction mixture was stirred at 45° C. for 24 h. The reaction mixture was diluted with 5 Ml of dichloromethane and 2 mL water. The organic layer was washed with water, dried (over Na2SO4) and concentrated. The crude was purified using silica gel column chromatography (2% MeOH—CH₂Cl₂) to yield 7 mg of tert-butyl (S)-1-(3-(5-(3-phenylureido)-3-(trifluoromethyl)-1H-pyrazol-1-yl)benzylamino)-1-oxopropan-2-ylcarbamate

The product was taken up in a mixture of trifluoroacetic acid and dichloromethane (50:200 Ul) and stirred at room temperature for 45 minutes. The reaction mixture was concentrated in vaccuo to furnish 7 mg of trifluoroacetic acid salt of 1-(1-(3-(((S)-2-aminopropanamido)methyl)phenyl)-3-(trifluoromethyl)-1H-pyrazol-5-yl)-3-phenylurea.

NMR (CD3OD): 7.61-7.47 (m, 4H), 7.35 (m, 2H), 7.27 (m, 2H), 7.03 (m, 1H), 6.83 (s, 1H), 4.53 (m, 2H), 3.92 (q, J=7.1 Hz, 1H), 1.50 (d, J=7.1 Hz, 3H).

MS m/z 447.4 (M+H)

### 3′-[((S)-2-Amino-propionylamino)-methyl]-biphenyl-2-carboxylic acid benzylamide (Example 190)

### Example 190

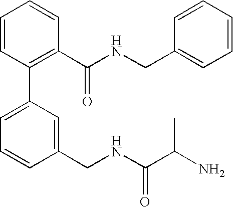

### Part A: Preparation of 3′-Aminomethyl-biphenyl-2-carboxylic acid methyl ester

Potassium carbonate (276 mg, 2 mmol) was added to the stirred suspension of 2-Iodo-benzoic acid methyl ester (157 MG, 0.6 mmol), 2-aminomethylphenylboronic acid (94 mg, 0.5 mmol) and Pd (0)(PPh3)4 (11.6 mg, 0.01 mmol) in a mixture of toluene-methanol-water (1.5:0.7:0.7 Ml). The reaction mixture was heated to 900 for 16 h. It was partitioned between water and dichloromethane (10 mL each). The aqueous layer was re-extracted with 10 mL of dichloromethane. Combined organic layer was washed with water; dried (Na2SO4) and concentrated to yield 32 mg of crude 3′-Aminomethyl-biphenyl-2-carboxylic acid methyl ester which was used as it in the next reaction.

MS m/z 242.3 (M+H)

### Part B: Preparation of 3′-[(2-tert-Butoxycarbonylamino-propionylamino)-methyl]-biphenyl-2-carboxylic acid methyl ester

Boc-Ala-Osu (40 mg, 0.14 mmol) was added to the solution of crude 3′-Aminomethyl-biphenyl-2-carboxylic acid methyl ester (29 mg, 0.12 mmol) and triethylamine (20 uL, 0.18 mmol) in dichloromethane (1 Ml). The reaction mixture was stirred at room temperature for 16 h. The reaction was diluted with 10 mL of dichloromethane and 2 mL of water. The organic layer was separated and washed with water, dried (Na2SO4) and concentrated to yield the crude which was purified using silica gel chromatography (25% EtOAc-hexane) to afford 30 mg of 3′-[(2-tert-Butoxycarbonylamino-propionylamino)-methyl]-biphenyl-2-carboxylic acid methyl ester.

NMR (CDCl3): 7.9 (m, 2H), 7.5 (d, 1H), 7.3 (m, 2H), 7.2 (m, 1H), 6.9 (m, 2H), 4.6 (bs, 2H), 4.19 (m, 1H), 3.6 (s, 3H), 1.4 (s, 9H), 1.3 (d, 3H)

### Part B: Preparation of 3′-[(2-tert-Butoxycarbonylamino-propionylamino)-methyl]-biphenyl-2-carboxylic acid

1 M aqueous solution of sodium hydroxide (0.36 mL) was added to the solution of 3′-[(2-tert-Butoxycarbonylamino-propionylamino)-methyl]-biphenyl-2-carboxylic acid methyl ester (30 mg, 0.073 mmol) in methanol (0.5 Ml). The solution was stirred at room temperature for 16 h and then concentrated in vaccuo. The residue was acidified with 10% acetic acid in water. The aqueous layer was diluted with ethyl acetate (5 Ml). The organic layer was separated and washed with water, dried (Na2SO4) and concentrated to furnish 22 mg of 3′-[(2-tert-Butoxycarbonylamino-propionylamino)-methyl]-biphenyl-2-carboxylic acid

MS m/z 399 (M+H)

### Part C: Preparation of 3′-[((S)-2-Amino-propionylamino)-methyl]-biphenyl-2-carboxylic acid benzylamide trifluoroacetate salt

EDCI (15 mg, 0.04 mmol) was added to the solution of 3′-[(2-tert-Butoxycarbonylamino-propionylamino)-methyl]-biphenyl-2-carboxylic acid (10 mg, 0.025 mmol), HOAt (11 mg, 0.04 mmol) and N,N-diisopropylethylamine (40 uL) in dichloromethane (0.3 Ml). After stirring at room temperature for 20 minutes, benzyl amine (5.4 mg, 0.05 mmol) was added and the reaction mixture was stirred at room temperature for additional 16 h. Diluted with 5 mL dichloromethane and 1 mL of water. The organic layer was washed with water, dried (Na2SO4) and concentrated. The crude was suspended in a mixture of trifluoroacetic acid and dichloromethane (0.1:0.3 Ml). The reaction mixture was stirred at room temperature for 30 minutes and then concentrated in vaccuo to afford the crude which was purified using the conditions below to furnish 10 mg of 3′-[((S)-2-Amino-propionylamino)-methyl]-biphenyl-2-carboxylic acid benzylamide trifluoroacetate salt
Conditions: Column—YMC ODS (20×50 mm)
Solvents—A—90% water—10% methanol—0.1% TFA
   B—10% water—90% methanol—0.1% TFA
Gradient—15% B to 100% B in 12 min
Retention time: 9.2 min

NMR (CD3OD): 7.54-7.20 (m, 11H), 7.02 (m, 2H), 4.46-4.33 (m, 4H), 3.92 (q, J=7.1 Hz, 1H), 1.50 (d, J=7.1 Hz, 3H)

MS m/z 388.5 (M+H)

### (2S)—N-(3-(5-(5-(2-methylbenzo [d]oxazol-7-yl)-1,3,4-oxadiazol-2-yl)-3-(trifluoromethyl)-1H-pyrazol-1-yl)benzyl)-2-aminopropanamide (Example 190)

### Example 190

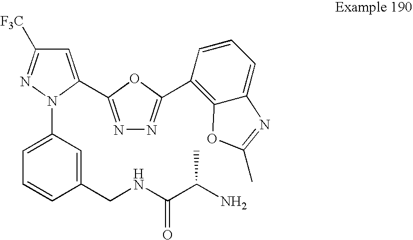

### Part A: Preparation of 2-methylbenzo[d]oxazole-7-carboxylic acid

A vial was charged with 3-amino-2-hydroxybenzoic acid (0.5 g, 3.26 mmol) in triethylorthoacetate (2 mL) and p-toluenesulfonic acid (20 mg). The reaction mixture was heated for 5 hours at 100° C. The reaction mixture was concentrated in vaccuo and the residue (0.55 g) was used as such in the next reaction.

MS m/z 178 (M+H)

### Part B: Preparation of (2S)—N-(3-(5-(5-(2-methylbenzo[d]oxazol-7-yl)-1,3,4-oxadiazol-2-yl)-3-(trifluoromethyl)-1H-pyrazol-1-yl)benzyl)-2-aminopropanamide

A vial containing 2-methylbenzo[d]oxazole-7-carboxylic acid (56.5 mg, 0.32 mmol), 1-(3-(((S)-2-(tert-butoxycarbonyl)propanamido)methyl)phenyl)-3-(trifluoromethyl)-1H-pyrazole-5-carboxylic acid hydrazide (100 mg, 0.21 mmol), PyBOP (165 mg, 0.31 mmol) and triethylamine (74 μL) in DMF (2 mL) was heated in microwave oven at 120° C. for 20 minutes.

The reaction mixture was cooled at room temperature and to which was added diisopropylcarbodidimide (134 mg, 1.06 mmol) and the reaction was heated to 140° C. for 30 minutes in a microwave oven. The reaction was diluted with methanol (2 mL) and directly loaded onto preparative HPLC column. The crude product was purified using the conditions as shown below to yield tert-butyl (S)-1-(3-(5-(5-(2-methylbenzo[d]oxazol-7-yl)-1,3,4-oxadiazol-2-yl)-3-(trifluoromethyl)-1H-pyrazol-1-yl)benzylamino)-1-oxopropan-2-ylcarbamate (10 mg)
Conditions: Column—YMC ODS (20×50 mm)
Solvents—A—90% water—10% methanol—0.1% TFA
   B—10% water—90% methanol—0.1% TFA
Gradient—25% B to 100% B in 12 min
Retention time: 9.6 min

tert-butyl (S)-1-(3-(5-(5-(2-methylbenzo[d]oxazol-7-yl)-1,3,4-oxadiazol-2-yl)-3-(trifluoromethyl)-1H-pyrazol-1-yl)benzylamino)-1-oxopropan-2-ylcarbamate (10 mg) was treated with a mixture of trifluoroacetic acid in dichloromethane (1:3, 2 Ml) for 2 hours at room temperature. Then the reaction mixture was concentrated in vacuo to yield (2S)—N-(3-(5-(5-(2-methylbenzo[d]oxazol-7-yl)-1,3,4-oxadiazol-2-yl)-3-(trifluoromethyl)-1H-pyrazol-1-yl)benzyl)-2-aminopropanamide (6 mg) as trifluoroacetate salt.

NMR (CD3OD): 8.2-8 (m, 1H), 7.8 (s, 1H), 7.7 (m, 1H), 7.52-7.4 (m, 2H), 7.3-7.1 (m, 2H), 4.37 (dd, 2H), 3.7 (q, J=7.1 Hz, 1H), 2.6 (s, 3H), 1.50 (d, J=7.1 Hz, 3H)

MS m/z 512.1 (M+H)

### (2S)—N-(3-(5-(3-(benzo [d]thiazol-7-yl)-1,2,4-oxadiazol-5-yl)-3-(trifluoromethyl)-1H-pyrazol-1-yl)benzyl)-2-aminopropanamide (Example 191)

### Example 191

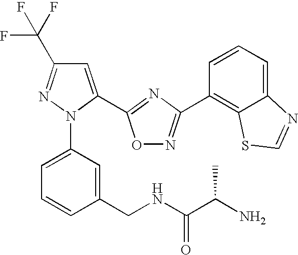

### Part A: Preparation of benzo[d]thiazole-7-carbaldehyde

Isobutyl chloroformate (715 mg, 5.5 mmol) was added to the cooled (0° C.) solution of benzo[d]thiazole-7-carboxylic acid (895 mg, 5 mmol) and N-methylmorpholine (1.2 mL, 11.0 mmol) in dichloromethane (15 mL). After 15 minutes of stirring, N-methoxymethanamine hydrochloride (488 mg, 5 mmol) was added and the reaction mixture was stirred at room temperature for 16 hours. The reaction mixture was washed with 0.2 M KHSO₄ (2×10 mL); followed by water and the combined organic layer was dried (Na₂SO₄) and concentrated. The product was used as such in the next reaction.

Di-isobutyl aluminium hydride (8 mL, 8.0 mmol, 1M soln in toluene) was added to the cooled solution of crude product as obtained above (890 mg, 4 mmol) in dry tetrahydrofuran (30 mL) cooled at −78° C. The reaction mixture was stirred at −78° C. for 1 hour and then quenched with 1 mL of ethyl acetate. The reaction mixture was poured into a mixture of ether and 1M aqueous citric acid. The layers were separated. The aquesous layer was extracted with ethyl acetate (20 mL). The combined organic layers were washed with water, dried (Na₂SO₄) and concentrated. The crude product was purified using silica gel column chromatography (30% ethyl acetate-hexane) to yield 325 mg of benzo[d]thiazole-7-carbaldehyde.

NMR (CDCl₃): 10.7(s, 1H), 9.75 (s, 1H), 8.3 (d, 1H), 7.9 (d, 1H), 7.63 (m, 1H)

MS m/z 164 (M+H)

### Part B: Preparation of benzo[d]thiazole-7-carbonitrile

A mixture of benzo[d]thiazole-7-carbaldehyde (200 mg, 1.2 mmol) and hydroxylamine hydrochloride (128 mg, 1.8 mmol) in a mixture of triethylamine and acetonitrile (260 μL: 10 mL) was heated at 70° C. for 2 hours. Solvent was evaporated under vacuo and the residue was used as such in the next reaction.

The crude product was treated with triphenylphosphine (390 mg, 1.5 mmol) and N-chlorosuccinamide (197 MG, 1.5 mmol) in dichloromethane (5 mL). The reaction mixture was stirred at room temperature for 16 hours and then concentrated in vacuo. The crude was purified using silica gel column chromatography (30% ethyl acetate-hexane) to afford 165 mg of benzo[d]thiazole-7-carbonitrile.

NMR (CDCl₃): 9.65 (s, 1H), 8.5 (d, 1H), 7.9 (d, 1H), 7.63 (m, 1H)

### Part C: Preparation of N′-hydroxybenzo[d]thiazole-7-carboxamidine

A mixture of benzo[d]thiazole-7-carbonitrile (20 mg, 0.13 mmol), hydroxylamine hydrochloride (13 mg, 0.19 mmol) and triethylamine (26.1 μL, 0.19 mmol) in ethanol (3 mL) was heated to 70° C. for 2 hours. The solvent was evaporated and the crude product was purified using preparative HPLC using conditions as described below to give 29 mg of N′-hydroxybenzo[d]thiazole-7-carboxamidine
Conditions: Column—YMC ODS (20×50 mm)
Solvents—A—90% water—10% methanol—0.1% TFA
   B—10% water—90% methanol—0.1% TFA
Gradient—15% B to 100% B in 12 min
Retention time: 8.6 min

MS m/z 194 (M+H)

### Part D: Preparation of (2S)—N-(3-(5-(3-(benzo[d]thiazol-7-yl)-1,2,4-oxadiazol-5-yl)-3-(trifluoromethyl)-1H-pyrazol-1-yl)benzyl)-2-aminopropanamide (Example 191)

A mixture of 1-(3-(((S)-2-(tert-butoxycarbonyl)propanamido)methyl)phenyl)-3-(trifluoromethyl)-1H-pyrazole-5-carboxylic acid (32 mg, 0.07 mmol), HBTU (27 mg, 0.07 mmol), N′-hydroxybenzo[d]thiazole-7-carboxamidine (13.5 mg, 0.07 mmol) and N,N′-diisopropylethylamine (29 μL, 0.17 mmol) in dry DMF (1 mL) was heated in microwave oven at 190° C. for 2 minutes. The reaction mixture was diluted with a mixture of ethyl acetate-water (20:5 mL); the organic layer was separated; washed with water; dried (Na₂SO₄) and concentrated to afford a crude.

The crude was treated with trifluoroacetic acid (0.2 mL) in dichloromethane (0.8 mL). The reaction mixture was concentrated, the resulting crude was dissolved in methanol and loaded onto preparative HPLC. The following conditions were used for the purification to yield 22 mg of (2S)—N-(3-(5-(3-(benzo[d]thiazol-7-yl)-1,2,4-oxadiazol-5-yl)-3-(trifluoromethyl)-1H-pyrazol-1-yl)benzyl)-2-aminopropanamide as trifluoroacetic acid salt.
Conditions: Column—YMC ODS (20×50 mm)
Solvents—A—90% water—10% methanol—0.1% TFA
   B—10% water—90% methanol—0.1% TFA
Gradient—15% B to 100% B in 12 min
Retention time: 9.4 min

NMR (CD3OD): 9.65 (s, 1H), 8.1-8.3 (m, 2H), 7.7-7.9 (m, 3H), 7.1 (m, 3H), 4.4 (dd, 2H), 3.8 (m, 1H), 1.3 (d, 3H)

MS m/z 514.2 (M+H)

We claim:

1. A compound of formula I, a stereoisomer, a tautomer, or a pharmaceutically acceptable salt thereof, 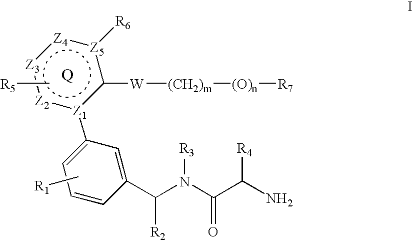
wherein:
Ring Q is phenyl; 5-membered heteroaryl, in which Z₄ is a bond, Z₁, Z₂, Z₃ and Z₅ are each independently C, N, O, or S, and at least one of Z₁, Z₂, Z₃ and Z₅ is a heteroatom selected from N, O and S; or 6-membered heteroaryl, in which Z₁, Z₂, Z₃, Z₄ and Z₅ are each independently C, or N, and at least one of Z₁, Z₂, Z₃, Z₄ and Z₅ is N;
W is —C(═O)NR₈—, —NR₉C(═O)—, —NR₉C(═O)NR₉—, 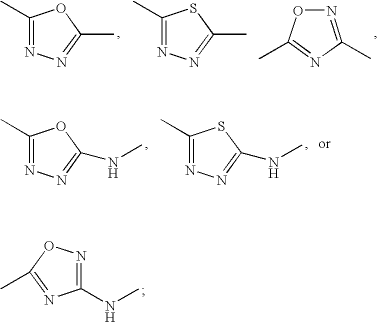
alternatively, W—(CH₂)—(O)n-R₇ is 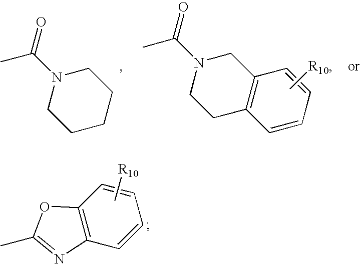
R₁ is H, halogen, CN, alkyl or substituted alkyl, O—C₁-C₄ alkyl, S—C₁-C₄ alkyl, or SO₂—C₁-C₄ alkyl;
R₂ is H or C₁-C₄ alkyl;
R₃ is H, Me or Et, or optionally R₃ together with R₄ may form a 5- or 6-membered heterocycle
R₄ is H, Me, Et, iso-propyl, CH₂Ph, OH, or OPh, or optionally R₄ together with R₃ may form a 5- or 6-membered heterocycle;
R₅ is nil, H, Me, Et, propyl, iso-propyl, OMe, OEt, SMe, SO₂Me, CF₃, or OCF₃;
R₆ is nil, H, Me, or Et, or optionally R₆ together with R₈ may form a 5- or 6-membered heterocycle;
R₇ is cycloalkyl or substituted cycloalkyl, heterocycle or substituted heterocycle, or aryl or substituted aryl;
R₈ is H, or Me, or optionally R₈ together with R₆ may form a 5- or 6-membered heterocycle;
or alternatively R₈ together with R₇ may form a 5- or 6-membered heterocycle or substituted heterocycle;
R₉ is H, or Me;
R₁₀ is hydrogen, halogen, haloalkyl, cyano, nitro, alkyl, cycloalkyl, alkenyl, cycloalkenyl, alkynyl, heterocycle, aryl, OR, SR, S(═O)R, S(═O)₂R, P(═O)₂R, S(═O)₂OR, P(═O)₂OR, NRR, NRS(═O)₂R, NRP(═O)₂R, S(═O)₂NRR, P(═O)₂NRR, C(═O)OR, C(═O)R, C(═O)NRR, OC(═O)R, OC(═O)NRR, NRC(═O)OR, NRC(═O)NRR, NRS(═O)₂NRR, NRP(═O)₂NRR, NR_{b}C(═O)Rₐ, or NRP(═O)₂R, wherein R is independently hydrogen, alkyl, cycloalkyl, alkenyl, cycloalkenyl, alkynyl, heterocycle, or aryl;
m is 0, 1, 2 or 3;
n is 0 or 1; and
p is 1, 2 or 3.

2. The compound of claim 1, wherein ring Q is 5-membered heteroaryl, in which Z₄ is a bond, Z₁, Z₂, Z₃ and Z₅ are each independently C, N, O, or S, and at least one of Z₁, Z₂, Z₃ and Z₅ is a heteroatom selected from N, O and S; or 6-membered heteroaryl, in which Z₁, Z₂, Z₃, Z₄ and Z₅ are each independently C, or N, and at least one of Z₁, Z₂, Z₃, Z₄ and Z₅ is N.

3. The compound of claim 2, wherein ring Q is 5-membered heteroaryl, in which Z₄ is a bond, Z₁, Z₂, Z₃ and Z₅ are each independently C, N, O, or S, and at least one of Z₁, Z₂, Z₃ and Z₅ is a heteroatom selected from N, O and S.

4. The compound of claim 3, wherein Z₄ is a bond, Z₁ and Z₂, are each independently N; Z₃ and Z₅ are each independently C.

5. The compound of claim 4, wherein R₇ is aryl or substituted aryl.

6. The compound of claim 5, wherein R₇ is phenyl or substituted phenyl.

7. The compound of claim 1, wherein W is —C(═O)NR₈—.

8. The compound of claim 7, wherein ring Q is 5-membered heteroaryl, in which Z₄ is a bond, Z₁, Z₂, Z₃ and Z₅ are each independently C, N, O, or S, and at least one of Z₁, Z₂, Z₃ and Z₅ is a heteroatom selected from N, O and S.

9. The compound of claim 8, wherein Z₄ is a bond, Z₁ and Z₂, are each independently N; Z₃ and Z₅ are each independently C.

10. The compound of claim 9, wherein R₇ is aryl or substituted aryl.

11. The compound of claim 10, wherein R₇ is phenyl or substituted phenyl.

12. The compound of claim 1, wherein W is

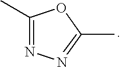

13. The compound of claim 12, wherein ring Q is 5-membered heteroaryl, in which Z₄ is a bond, Z₁, Z₂, Z₃ and Z₅ are each independently C, N, O, or S, and at least one of Z₁, Z₂, Z₃ and Z₅ is a heteroatom selected from N, O and S.

14. The compound of claim 13, wherein Z₄ is a bond, Z₁ and Z₂, are each independently N; Z₃ and Z₅ are each independently C.

15. The compound of claim 14, wherein R₇ is aryl or substituted aryl.

16. The compound of claim 15, wherein R₇ is phenyl or substituted phenyl.

17. The compound of claim 1, wherein R₂ is H, R₃ is H, R₄ is Me, Et, OH, or Ph.

18. The compound of claim 17, wherein R₄ is Me.

19. The compound of claim 18, wherein R₄ is (S—) Me.

20. The compound of claim 19, wherein ring Q is 5-membered heteroaryl, in which Z₄ is a bond, Z₁, Z₂, Z₃ and Z₅ are each independently C, N, O, or S, and at least one of Z₁, Z₂, Z₃ and Z₅ is a heteroatom selected from N, O and S.

21. The compound of claim 20, wherein Z₄ is a bond, Z₁ and Z₂, are each independently N; Z₃ and Z₅ are each independently C.

22. The compound of claim 19, wherein W is —C(═O)NR₈—.

23. The compound of claim 22, wherein ring Q is 5-membered heteroaryl, in which Z₄ is a bond, Z₁, Z₂, Z₃ and Z₅ are each independently C, N, O, or S, and at least one of Z₁, Z₂, Z₃ and Z₅ is a heteroatom selected from N, O and S.

24. The compound of claim 23, wherein Z₄ is a bond, Z₁ and Z₂, are each independently N; Z₃ and Z₅ are each independently C.

25. The compound of claim 19, wherein W is

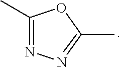

26. The compound of claim 25, wherein ring Q is 5-membered heteroaryl, in which Z₄ is a bond, Z₁, Z₂, Z₃ and Z₅ are each independently C, N, O, or S, and at least one of Z₁, Z₂, Z₃ and Z₅ is a heteroatom selected from N, O and S.

27. The compound of claim 26, wherein Z₄ is a bond, Z₁ and Z₂, are each independently N; Z₃ and Z₅ are each independently C.

28. A pharmaceutical composition comprising at least one compound according to claim 1 and a pharmaceutically-acceptable carrier or diluent.

29. A pharmaceutical composition of claim 28, further comprising at least one other anti-cancer agent or cytotoxic agent.

30. The pharmaceutical composition of claim 29, wherein said anti-cancer or cytotoxic agent is selected from the group consisting of tamoxifen, toremifene, raloxifene, droloxifene, iodoxifene, megestrol acetate, anastrozole, letrozole, borazole, exemestane, flutamide, nilutamide, bicalutamide, cyproterone acetate, gosereline acetate, leuprolide, finasteride, metalloproteinase inhibitors, inhibitors of urokinase plasminogen activator receptor function, growth factor antibodies, growth factor receptor antibodies, bevacizumab, cetuximab, tyrosine kinase inhibitors, serine/threonine kinase inhibitors, methotrexate, 5-fluorouracil, purine and adenosine analogues, cytosine arabinoside, doxorubicin, daunomycin, epirubicin, idarubicin, mitomycin-C, dactinomycin, mithramycin, cisplatin, carboplatin, nitrogen mustard, melphalan, chlorambucil, busulphan, cyclophosphamide, ifosfamide, nitrosoureas, thiotepa, vincristine, vinorelbine, vinblastine, vinflunine, paclitaxel, docetaxel, epothilone analogs, discodermolide analogs, eleutherobin analogs, etoposide, teniposide, amsacrine, topotecan, flavopyridols, proteasome inhibitors including bortezomib and biological response modifiers, androgen receptor antagonists, LH/RH antagonists, taxane analogues, and estrogen receptor antagonists.

31. A method of inhibiting the activity of CARM-1 which comprises administering to a mammalian species in need thereof an effective amount of at least one compound of formula I: 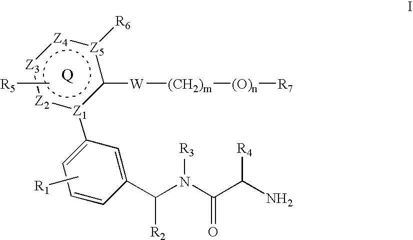
or a stereoisomer, a tautomer, a pharmaceutically acceptable salt or solvate thereof,
wherein:
Ring Q is phenyl; 5-membered heteroaryl, in which Z₄ is a bond, Z₁, Z₂, Z₃ and Z₅ are each independently C, N, O, or S, and at least one of Z₁, Z₂, Z₃ and Z₅ is a heteroatom selected from N, O and S; or 6-membered heteroaryl, in which Z₁, Z₂, Z₃, Z₄ and Z₅ are each independently C, or N, and at least one of Z₁, Z₂, Z₃, Z₄ and Z₅ is N;
W is —C(═O)NR₈—, —NR₉C(═O)—, 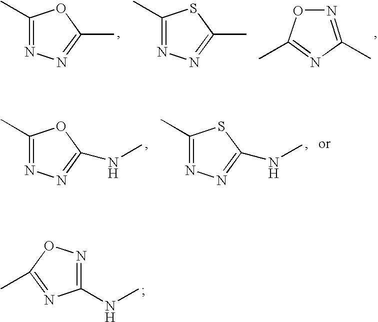
R₁ is H, halogen, CN, alkyl or substituted alkyl, O—C₁-C₄ alkyl, S—C₁-C₄ alkyl, or SO₂—C₁-C₄ alkyl;
R₂ is H or C₁-C₄ alkyl;
R₃ is H, Me or Et, or optionally R₃ together with R₄ may form a 5- or 6-membered heterocycle
R₄ is H, Me, Et, iso-propyl, CH₂Ph, OH, or OPh, or optionally R₄ together with R₃ may form a 5- or 6-membered heterocycle;
R₅ is nil, H, Me, Et, propyl, iso-propyl, OMe, OEt, SMe, SO₂Me, CF₃, or OCF₃;
R₆ is nil, H, Me, or Et, or optionally R₆ together with R₈ may form a 5- or 6-membered heterocycle;
R₇ is cycloalkyl or substituted cycloalkyl, heterocycle or substituted heterocycle, or aryl or substituted aryl;
R₈ is H, or Me, or optionally R₈ together with R₆ may form a 5- or 6-membered heterocycle;
or alternatively R₈ together with R₇ may form a 5- or 6-membered heterocycle or substituted heterocycle;
R₉ is H, or Me;
m is 0, 1, 2 or 3;
n is 0 or 1; and
p is 1, 2 or 3.

32. A method for treating a condition or disorder comprising administering to a mammalian species in need thereof a therapeutically effective amount of at least one compound of formula I: 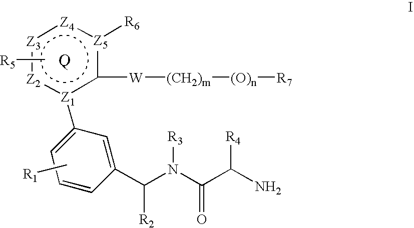
or a stereoisomer, a tautomer, a pharmaceutically acceptable salt or solvate thereof,
wherein:
Ring Q is phenyl; 5-membered heteroaryl, in which Z₄ is a bond, Z₁, Z₂, Z₃ and Z₅ are each independently C, N, O, or S, and at least one of Z₁, Z₂, Z₃ and Z₅ is a heteroatom selected from N, O and S; or 6-membered heteroaryl, in which Z₁, Z₂, Z₃, Z₄ and Z₅ are each independently C, or N, and at least one of Z₁, Z₂, Z₃, Z₄ and Z₅ is N;
W is —C(═O)NR₈—, —NR₉C(═O)—, 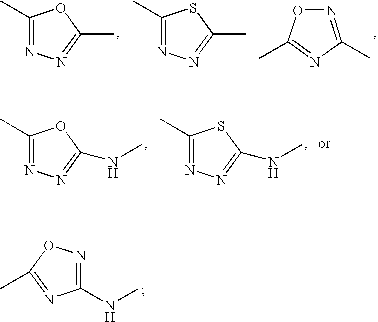
R₁ is H, halogen, CN, alkyl or substituted alkyl, O—C₁-C₄ alkyl, S—C₁-C₄ alkyl, or SO₂—C₁-C₄ alkyl;
R₂ is H, or C₁-C₄ alkyl;
R₃ is H, Me or Et, or optionally R₃ together with R₄ may form a 5- or 6-membered heterocycle
R₄ is H, Me, Et, iso-propyl, CH₂Ph, OH, or OPh, or optionally R₄ together with R₃ may form a 5- or 6-membered heterocycle;
R₅ is nil, H, Me, Et, propyl, iso-propyl, OMe, OEt, SMe, SO₂Me, CF₃, or OCF₃;
R₆ is nil, H, Me, or Et, or optionally R₆ together with R₈ may form a 5- or 6-membered heterocycle;
R₇ is cycloalkyl or substituted cycloalkyl, heterocycle or substituted heterocycle, or aryl or substituted aryl;
R₈ is H, or Me, or optionally R₈ together with R₆ may form a 5- or 6-membered heterocycle;
or alternatively R₈ together with R₇ may form a 5- or 6-membered heterocycle or substituted heterocycle;
R₉ is H, or Me;
m is 0, 1, 2 or 3;
n is 0 or 1; and
p is 1, 2 or 3; and
wherein said condition or disorder is selected from the group consisting of proliferate diseases, cancers, benign prostate hypertrophia, benign prostatic hyperplasia, adenomas and neoplasies of the prostate, benign or malignant tumor cells containing the androgen receptor, brain cancer, skin cancer, bladder cancer, lymphatic cancer, liver cancer, kidney cancer, pancreatic cancer, prostate cancer, hirsutism, acne, precocious puberty, angiogenic conditions or disorders, hyperpilosity, inflammation, immune modulation, seborrhea, endometriosis, polycystic ovary syndrome, androgenic alopecia, hypogonadism, osteoporosis, suppressing spermatogenesis, male and female sexual dysfunction, libido, cachexia, anorexia, inhibition of muscular atrophy in ambulatory patients, androgen supplementation for age related decreased testosterone levels in men, cancers expressing the estrogen receptor, breast cancer, ovarian cancer, uterine cancer, endometrial cancer, hot flushes, vaginal dryness, menopause, amennoreahea, dysmennoreahea, contraception, pregnancy termination, cancers containing the progesterone receptor, cyclesynchrony, meniginoma, fibroids, labor induction, autoimmune diseases, Alzheimer's disease, psychotic disorders, drug dependence, non-insulin dependent Diabetes Mellitus, dopamine receptor mediated disorders, heart disease, congestive heart failure, disregulation of cholesterol homeostasis, and attenuating the metabolism of a pharmaceutical agent.

33. A method for treating cancer comprising administering to a patient in need thereof, a therapeutically effective amount of a compound having the formula I according to claim 1, wherein the cancer is breast cancer, lung cancer, ovarian cancer, prostate cancer, leukemia, lymphoma, glioblastoma, brain cancer, melanoma, or colon cancer.
